(19) United States
(12) Patent Application Publication
(10) Pub. No.: US 2020/0316062 A1
(43) Pub. Date: Oct. 8, 2020

(54) COMBINATIONS COMPRISING HISTONE DEACETYLASE INHIBITORS

(71) Applicant: Karus Therapeutics Limited, Oxfordhire (GB)

(72) Inventors: Stephen Joseph Shuttleworth, Oxfordshire (GB); Andrew David Whale, Oxfordshire (GB); Lucy Mary Colman, Oxfordshire (GB); Helen Louise Rogers, Oxfordshire (GB)

(21) Appl. No.: 16/304,789

(22) PCT Filed: Jun. 5, 2017
(86) PCT No.: PCT/GB2017/051619

§ 371 Date: Nov. 27, 2018

(30) Foreign Application Priority Data
Jun. 3, 2016 (GB) 1609786.7

(51) Int. Cl. A61K 31/497; A61K 31/69; A61K 31/5377; C07K 16/28; A61K 31/4439; A61P 35/00

(52) U.S. Cl. CPC A61K 31/497; A61K 31/69; A61K 31/5377; A61K 45/06; A61K 31/4439; A61P 35/00; C07K 16/2818

(57) ABSTRACT

The invention relates to a combination of a compound of formula (I) or a pharmaceutically acceptable salt thereof, and at least one second agent selected from the group consisting of proteasome inhibitors, tumour immunotherapeutics or immunomodulatory agents, signal transduction pathway inhibitors, agents inhibiting the BCL2 family of proteins, agents inhibiting Mcl-1, poly (ADP-ribose) polymerase (PARP) Inhibitors, aromatase inhibitors, conventional cytotoxic agents or a miscellaneous agent selected from abiraterone, ARN-509 and MYC inhibitors.

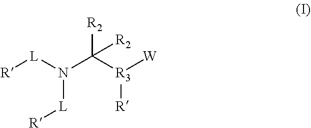

### FIELD OF THE INVENTION

The present invention relates to novel combinations comprising a compound which acts as an inhibitor of histone deacetylase (HDAC), in combinations with other specific anti-tumour compounds. Such combinations are useful in the therapy of cancer.

### BACKGROUND OF THE INVENTION

HDACs are zinc metalloenzymes that catalyse the hydrolysis of acetylated lysine residues. In histones, this returns lysines to their protonated state and is a global mechanism of eukaryotic transcriptional control, resulting in tight packaging of DNA in the nucleosome. Additionally, reversible lysine acetylation is an important regulatory process for non-histone proteins. Thus, compounds which are able to modulate HDAC have important therapeutic potential.

### SUMMARY OF THE INVENTION

The present invention relates in part to combinations of certain HDAC inhibitors and certain other anti-tumour compounds. These combinations may be synergistic and therefore may offer improvements with respect to the individual components. For example, they may allow a lower dose to be administered. The present invention is based in part on the data presented herein.

Certain HDAC inhibitors disclosed herein are also disclosed in WO 2014/181137.

The present invention is directed in part to a combination of certain HDAC inhibitors with certain anti-tumour agents.

Therefore, the present invention is a pharmaceutical composition comprising an HDAC inhibitor of Formula (I):

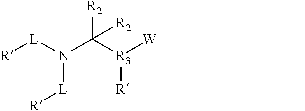

or a pharmaceutically acceptable salt thereof, wherein:

each R^{/} is independently selected from H and QR₁;

each Q is independently selected from a bond, CO, CO₂, NH, S, SO, SO₂ or O;

each R₁ is independently selected from H, C₁-C₁₀ alkyl, C₂-C₁₀ alkenyl, C₂-C₁₀ alkynyl, aryl, heteroaryl, C₁-C₁₀ cycloalkyl, halogen, C₁-C₁₀ alkylaryl, C₁-C₁₀ alkyl heteroaryl or C₁-C₁₀ heterocycloalkyl;

each L is independently selected from a 5 to 10-membered nitrogen-containing heteroaryl;

W is a zinc-binding group;

each R₂ is independently hydrogen or C₁ to C₆ alkyl; and

R₃ is an aryl or heteroaryl;

each aryl or heteroaryl may be substituted by up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃-acylamino, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl; and

each alkyl, alkenyl or alkynyl may be substituted with halogen, NH₂, NO₂ or hydroxyl;

in combination with

at least one agent selected from the group consisting of signal transduction pathway inhibitors, tumour immunotherapeutics, agents inhibiting the BCL2 family of proteins, agents inhibiting Mc-1, proteasome Inhibitors, poly (ADP-ribose) polymerase (PARP) Inhibitors, aromatase inhibitors, conventional cytotoxic agents or a miscellaneous agent selected from abiraterone, ARN-509 and MYC inhibitors.

### DESCRIPTION OF THE PREFERRED EMBODIMENTS

### Definitions

As used herein, “alkyl” means a C₁-C₁₀ alkyl group, which can be linear or branched. Preferably, it is a C₁-C₆ alkyl moiety. More preferably, it is a C₁-C₄ alkyl moiety. Examples include methyl, ethyl, n-propyl and t-butyl. It may be divalent, e.g. propylene.

As used herein, “cycloalkyl” contains from 3 to 10 carbon atoms. It may be monovalent or divalent.

As used herein, “alkenyl” means a C₂-C₁₀ alkenyl group. Preferably, it is a C₂—C alkenyl group. More preferably, it is a C₂-C₄ alkenyl group. The alkenyl radicals may be mono- or di-saturated, more preferably monosaturated. Examples include vinyl, allyl, 1-propenyl, isopropenyl and 1-butenyl. It may be divalent, e.g. propenylene

As used herein, “alkynyl” is a C₂-C₁₀ alkynyl group which can be linear or branched. Preferably, it is a C₂-C₄ alkynyl group or moiety. It may be divalent.

Each of the C₁-C₁₀ alkyl, C₂-C₁₀ alkenyl and C₂-C₁₀ alkynyl groups may be optionally substituted with each other, i.e. C₁-C₁₀ alkyl optionally substituted with C₂-C₁₀ alkenyl. They may also be optionally substituted with aryl, cycloalkyl (preferably C₃-C₁₀), aryl or heteroaryl. They may also be substituted with halogen (e.g. F, Cl), NH₂, NO₂ or hydroxyl. Preferably, they may be substituted with up to 10 halogen atoms or more preferably up to 5 halogens. For example, they may be substituted by 1, 2, 3, 4 or 5 halogen atoms. Preferably, the halogen is fluorine. For example, C₁-C₁₀ alkyl may be CF₃, CHF₂, CH₂CF₃, CH₂CHF₂ or CF₂CF₃ or OCF₃, OCHF₂, OCH₂CF₃, OCH₂CHF₂ or OCF₂CF₃.

As used herein, “aryl” means a monocyclic, bicyclic, or tricyclic monovalent or divalent (as appropriate) aromatic radical, such as phenyl, biphenyl, naphthyl, anthracenyl, which can be optionally substituted with up to three substituents preferably selected from the group of C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃-acylamino, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —S₃H, C₁-C₃ alkylsulfonyl, aminosulfonyl, mono C₁-C₃ alkyl aminosulfonyl and bis C₁-C₃-alkyl aminosulfonyl.

Amino means —NH₂.

As used herein, heteroaryl means a monocyclic, bicyclic or tricyclic monovalent or divalent (as appropriate) aromatic radical containing up to four heteroatoms selected from oxygen, nitrogen and sulfur, such as thiazolyl, tetrazolyl, imidazolyl, oxazolyl, isoxazolyl, thienyl, pyrazolyl, pyridinyl, pyrazinyl, pyrimidinyl, indolyl, quinolyl, isoquinolyl, said radical being optionally substituted with up to three substituents preferably selected from the group of C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis (C₁-C₃ alkyl) amino C₁-C₃ alkyl, C₁-C₃-acylamino, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

In the compounds of the invention, certain heteroaryl groups (i.e. L and R₃) are attached to R′. However, they may still be substituted by up to three additional substituents, selected from the groups defined above. Preferably, R′ is the only substituent.

As used herein, the term heterocycle or heterocycloalkyl is a mono- or di-valent carbocyclic radical containing up to 4 heteroatoms selected from oxygen, nitrogen and sulfur. It may be bicyclic or monocyclic. It is preferably saturated. The word ‘linker’ has been used herein to mean di-valent. If the heterocycle is a di-valent linker, the heterocycle may be attached to neighbouring groups through a carbon atom, or through on of the heteroatoms, e.g. a N. Examples of heterocycles are piperazine and morpholine.

The heterocyclic ring may be mono- or di-unsaturated. The radical may be optionally substituted with up to three substituents independently selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis (C₁-C₃ alkyl) amino C₁-C₃ alkyl, C₁—C-acylamino, C₁-C₃ alkyl sulfonylamino, halo e.g. F, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

As used herein, the above groups can be followed by the suffix-ene. This means that the group is divalent, i.e. a linker group.

As used herein, “thiol-protecting group” is typically:

(a) a protecting group that forms a thioether to protect a thiol group, for example a benzyl group which is optionally substituted by C₁-C₆ alkoxy (for example methoxy), C₁-C₆ acyloxy (for example acetoxy), hydroxy and nitro, picolyl, picolyl-N-oxide, anthrylmethyl, diphenylmethyl, phenyl, t-butyl, adamantyl, C₁-C₆ acyloxymethyl (for example pivaloyloxymethyl, tertiary butoxycarbonyloxymethyl);

(b) a protecting group that forms a monothio, dithio or aminothioacetal to protect a thiol group, for example C₁-C₆ alkoxymethyl (for example methoxymethyl, isobutoxymethyl), tetrahydropyranyl, benzylthiomethyl, phenylthiomethyl, thiazolidine, acetamidemethyl, benzamidomethyl;

(c) a protecting group that forms a thioester to protect a thiol group, such as tertiary-butyloxycarbonyl (BOC), acetyl and its derivatives, benzoyl and its derivatives; or

(d) a protecting group that forms a carbamic acid thioester to protect a thiol group, such as carbamoyl, phenylcarbamoyl, C₁-C₆ alkylcarbamoyl (for example methylcarbamoyl and ethylcarbamoyl).

### Preferred Groups of the Invention—Compounds of Formula (I)

Preferably, at least one R₂ is H. Preferably, both R₂ groups are H.

The group W is a zinc-chelating residue, i.e. a metallophile capable of binding with zinc in the active site of HDAC. Suitable metallophiles are known to those skilled in the art.

In a preferred embodiment, W is selected from:

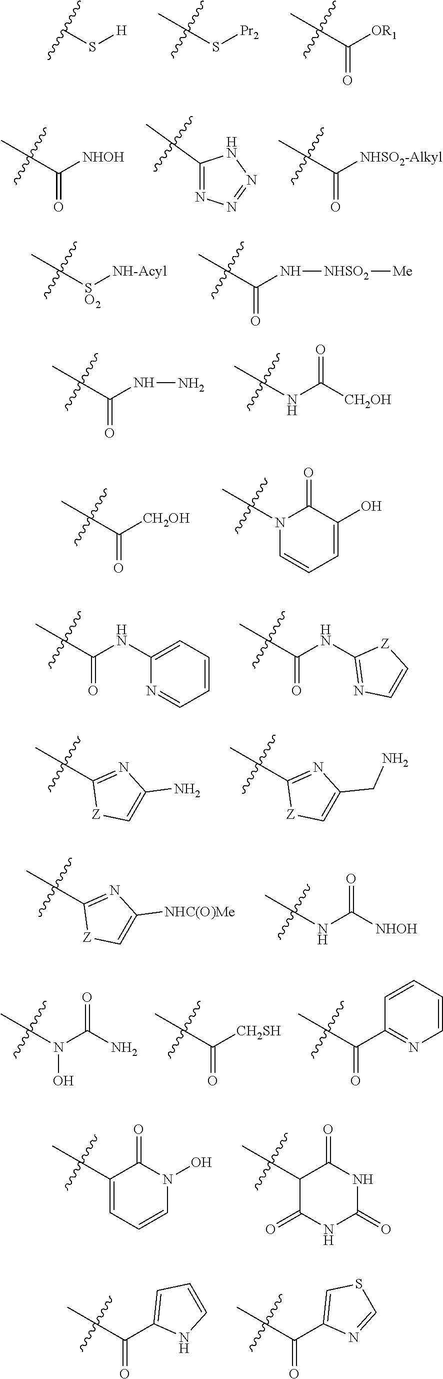

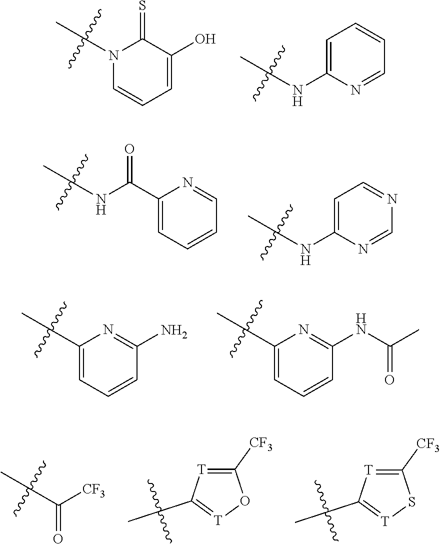

wherein R₁ is as defined in claim 1, Pr² is H or a thiol protecting group, Z is selected from O, S or NH and T is N or CH.

When W is COOR₁, preferably R₁ is not halogen. More preferably, when W is COOR₁, R₁ is H or C₁-C₁₀ alkyl.

Preferably, W is —COOH, —CONHOH, CONHS₂CH₃, —CONHNHSO₂CH₃, —CONHNH₂, —CONH(2-pyridyl), —NHCONHOH, tetrazole, hydroxypyridin-2-thione or hydroxypyridin-2-one. Preferably W is not COOR₁. More preferably, W is COOMe, —CONHOH, CONHSO₂CH₃, —CONHNHSO₂CH₃, —CONHNH₂, —CONH(2-pyridyl) —NHCONHOH, tetrazole, hydroxypyridin-2-thione or hydroxypyridin-2-one. Even more preferably, W is —CONHOH, tetrazole, hydroxypyridin-2-thione or hydroxypyridin-2-one. Most preferably, W is —CONHOH.

In a preferred embodiment, in at least one, preferably both L groups, the atom that is directly bonded to X is a carbon, and at least one nitrogen atom is directly bonded to said carbon.

In an embodiment, at least one L group is a 5-membered heteroaryl.

Preferably, at least one L group is a 6-membered heteroaryl. Even more preferably, both L groups are a 6-membered heteroaryl.

Preferably, at least one L group is pyridinyl, pyrimidinyl, pyridazinyl, oxadiazolyl, pyrazolyl, thiadiazolyl, pyrazinyl, benzofused thiazolyl, benzofused oxazolyl or benzofused imidazolyl. More preferably, at least one L group is pyridyl or pyrazinyl. Most preferably, one L is pyrazinyl and one L is pyridyl. Preferably, when L is pyridyl, it is substituted with a heteroaryl group. The heteroaryl group is preferably an optionally substituted (preferably substituted) pyridine.

Preferably, at least one L group is pyridinyl, oxadiazolyl, pyrazolyl, thiadiazolyl, pyrazinyl, benzofused thiazolyl, benzofused oxazolyl or benzofused imidazolyl.

Preferably, at least one L group is a 5 or 6-membered heteroaryl, which is optionally fused to a benzene.

Preferably, Q is a bond or 0.

Preferably, R₃ is aryl. More preferably, R₃ is phenylene or phenylene substituted with a halogen.

Preferably, at least one, preferably both, R₂ is H.

In a preferred embodiment, at least one R′ is H, halogen, CF₃, C₁-C₆ alkyl, aryl optionally substituted with halogen or heteroaryl optionally substituted with halogen. Preferably, the alkyl is substituted with at least one halogen, which is preferably fluorine.

In a preferred embodiment, the R′ attached to R₃ is hydrogen or halogen.

Preferably, R₃ is hydrogen or fluorine. More preferably, the R′ attached to R₃ is hydrogen. In a preferred embodiment, at least one R′, and preferably at least one of the R′ that is attached to L, is H, C₁-C₁₀ alkyl or O—(C₁-C₁₀ alkyl). Preferably, at least one R^{/} is substituted or unsubstituted aryl or O-(substituted or unsubstituted aryl). Preferably, at least one R^{/} is aryl or O-aryl, each of which may be substituted with a halogen, amino or C₁-C₁₀ alkyl. The aryl may be substituted in any position. The aryl may be mono-, bis-, or tri-substituted.

In a preferred embodiment, at least one R′, and preferably at least one of the R′ that is attached to L, is H, C₁-C₁₀ alkyl or O—(C₁-C₁₀ alkyl), halogen, C₁-C₁₀ heterocycloalkyl, aryl (preferably optionally substituted phenyl), trifluoromethyl or heteroaryl, preferably heteroaryl. Preferably, when R′ is heteroaryl, it is optionally substituted pyridyl, preferably a substituted pyridyl.

In one embodiment, at least one R′ that is attached to L is OCH₃ or CH₃. Preferably, at least one of the R′ that is attached to L is heterocycloalkyl. Preferably, the heterocycloalkyl is morpholino.

In a preferred embodiment, when Q is a direct bond, R₁ is H, C₁-C₁₀ alkyl or O—(C₁-C₁₀ alkyl), halogen (preferably F), C₁-C₁₀ heterocycloalkyl (preferably morpholino), aryl (preferably optionally substituted phenyl), trifluoromethyl or heteroaryl, preferably heteroaryl. Preferably, when R₁ is heteroaryl, it is optionally substituted pyridyl, preferably a substituted pyridyl.

In a preferred embodiment, R₁ is C₁-C₁₀ alkyl, C₂-C₁₀ alkenyl or C₂-C₁₀ alkynyl, preferably those groups are substituted with halogen, NH₂, NO₂ or hydroxyl.

More preferably, when R^{/} or R₁ is C₁-C₁₀ alkyl, it may be substituted with halogen which is preferably fluorine. The C₁-C₁₀ alkyl group may be substituted by up to 10 halogen atoms or preferably, by up to 5 halogen atoms, i.e., 1, 2, 3, 4 or 5 halogen atoms. For example, R^{/} or R₁ may be CF₃, CHF₂, CH₂CF₃, CH₂CHF₂ or CF₂CF₃ or OCF₃, OCHF₂, OCH₂CF₃, OCH₂CHF₂ or OCF₂CF₃.

R^{/} may be substituted onto any of the ring atoms of the L group or onto any of the ring atoms of the R₂ group.

Preferably, the L and R₃ groups have no other substitutions other than R′.

Preferably, Q is a direct bond.

Preferably, in addition to a N atom, L contains at least one other heteroatom in the heteroaryl ring which is selected from N, O or S.

In a preferred embodiment, L is:

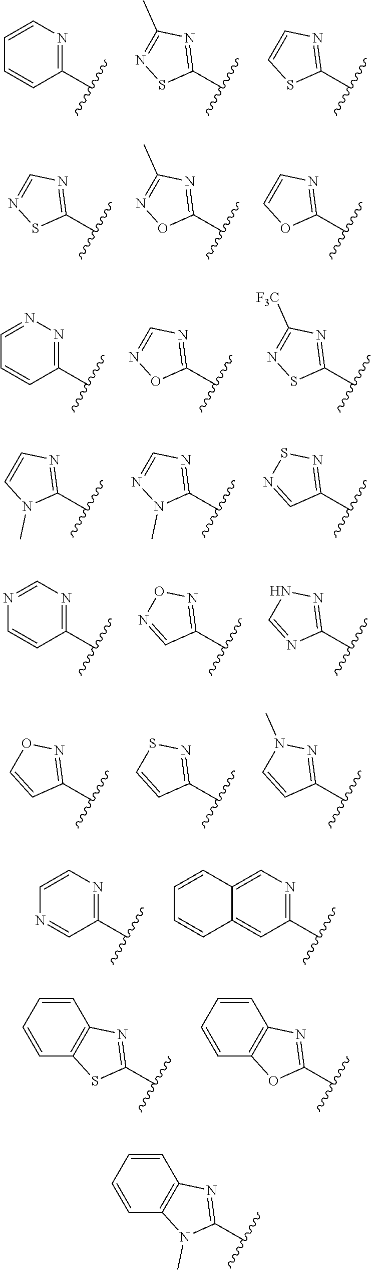

In a preferred embodiment, L is a hydrogen bond-acceptor, and preferably not also a hydrogen bond donor. Preferably, L does not have a hydrogen atom attached to an electronegative atom, such as N or O.

The definition of hydrogen bond acceptors/donors is known to those skilled in the art. For example, a hydrogen bond donor will have a hydrogen attached to an electronegative atom, such as N or O. For example, a hydrogen bond acceptor will have a N or O, which has a free lone pair.

Preferably the atom of L that is directly bonded to the N atom of the formula of claim 1 is carbon, and at least one nitrogen atom is directly bonded to said carbon (preferably via a double bond). More preferably, said nitrogen atom is a hydrogen bond acceptor.

For example, provided herein are HDAC inhibitors represented by:

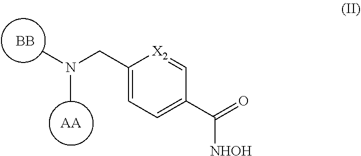

AA is monocyclic 5-6 membered heteroaryl or 8-10 membered bicyclic heteroaryl, where AA has at least one nitrogen, and optionally one or more additional heteroatoms;

BB is a monocyclic 5-6 membered heteroaryl having one or two nitrogens;

X² is N or CR¹²;

R¹² is hydrogen or halogen;

wherein AA or BB is optionally substituted by a substituent each independently selected from the group consisting of halogen, C₁₋₄alkyl, C₁₋₄alkoxy, phenyl, pyridinyl, and NR¹³R¹⁴;

R¹³ and R¹⁴ are each selected from the group consisting of H and C¹⁻⁴alkyl, or R¹³ and R¹⁴ taken together with the nitrogen to which they are attached form a 5-6 membered heterocycle optionally having an additional heteroatom;

wherein C₁₋₄alkyl, C₁₋₄alkoxy, phenyl or pyridinyl, for each occurrence, may each be optionally substituted by a substituent selected from the group consisting of one, two or three halogens; NR^{a}R^{b}, where R^{a} and R^{b} are each H or C₁₋₃alkyl.

### Preferred Combination Agents of the Invention

A HDAC inhibitor of formula (I) (e.g., formula (II) or as disclosed herein) may be combined with a signal transduction pathway inhibitor.

In some embodiments, the signal transduction pathway inhibitor is selected from the list below:
i. Bruton's tyrosine kinase (BTK) inhibitors (e.g. Ibrutinib, CC-292, CNX-774, CG11746, LFM-A13, RN486);
ii. Spleen tyrosine kinase (SYK) inhibitors (e.g. R788 (Fostamatinib), R406, GS-9973, Piceatannol, PRT062607);
iii. BMX non-receptor tyrosine kinase inhibitors; BMX is a member of the Tec family of kinases. Inhibitors include BMX-IN-1;
iv. Anaplastic lymphoma kinase (ALK) inhibitors (e.g. Ceritinib, Crizotinib, TAE684, AP26113, Alectinib, PF-06463922, GSK1838705A, AZD3463, ASP3016;
v. Small molecule inhibitors of—and biological agents targeting—tyrosine kinases including growth factor receptor tyrosine kinases, such as:
   i. the epidermal growth factor receptor (EGFR) (e.g. Trastuzumab, Cetixumab, Panitumumab, Zalutumumab, Nimotuzumab, Matuzumab, Gefitinib, Erlotinib, Lapatinib, AP26113);
   ii. the platelet-derived growth factor receptor (PDGFR) (e.g. Sorafenib, Sunitinib, Cabozantinib, Axitinib, AZD2932, Dovitinib, LY2874455, Foretinib, Vandetanib, SKLB1002, BMS-794833, Ki8751, Apatinib, AEE788, Tivozanib, Brivanib, ENMD-2076, Lenvatinib, OSI-930, Pazopanib, RAF265, CYC116, PD173074, PD173074, KRN633, Cabozantinib, ZM306416, Golvatinib, ZM323881, Semaxanib, SAR131675, MGCD-265, Orantinib, Vantanalib, Cediranib, Regorafenib);
   iii. the fibroblast growth factor receptor (FGFR) (e.g. Ponatinib, BGJ398, Nintedanib, PD173074, CH5183284, LY2874455, AZD4547, Danusertib, Tyrphostin, SSR128129E, MK-2461, Brivanib, TSU-68);
   iv. the vascular endothelial growth factor receptor (VEGFR) (e.g. Cabozantinib, PD153035).
vi. Vascular endothelial growth factor (VEGF) inhibitors (e.g. Bevacizumab, Ranibizumab).
vii. Small molecule inhibitors of the ribosomal protein S6 kinase, p-70S6K (e.g. LY2584702, BI-D1870, PF-4708671, AT7867, AT13148).
viii. Inhibitors of mammalian target of rapamycin (mTOR) (e.g. Sirolimus, Everolimus, AZD8055, Temsirolimus, MHY1485, Zotarolimus, KU-0063794, ETO-46464, GDC-0349, XL388, WYE-354, WYE-125132, WAY-600, WYE-687, PP121, AZD2014, INK128, Voxtalisib, Ridaforolimus, Torkinib, OSI-027, Palomid 529).
ix. RAF kinase inhibitors (e.g. Vemurafenib, Dabrafenib, Sorafenib, PLX-4720, LY3009120, RAF265, AZ638, Encorafenib, GDC-0879, CEP-32496, TAK-632, ZM-336372, NVP-BHG712, SB590885, GW5074);
j. Mitogen-activated protein kinase (MEK) inhibitors (e.g. Trametinib, Selumetinib, PD0325901, U0126, PD184352, GDC-0623, BI-847325, Cobimetinib, PD98059, BIX-02189, Binimetinib, Pimasertib, CL-327, AZD8330, TAK-733, PD318088, Redametinib);
k. BCR-ABL inhibitors (e.g. Imatinib, Dasatinib, Saracatinib, Nilotinib, Ponatinib, PD173955, Danusertib, AT9283, GNF-5, GZD824, KW-2449, DCC-2036, NVP-BHG712, GNF-2, Baferinib, Degrasyn);
l. Extracellular signal-regulated kinase (ERK) inhibitors (e.g. SCH772984, XMD8-92, FR-180204, GDC-0994, ERK5-IN-1, Ulixertinib);
m. JAK-STAT signalling inhibitors (e.g. Pacritinib, Tofacitinib, AZD1480, Ruxolitinib, Fedratinib, AT9283, Cerdulatinib, Filgotinic, Go6976, AG-490, Momelotinib, GLPG0634, ZM039923, ZL019, Curcumol, CEP-33779, AZ-960, TG1011209, NVP-BSK805, Baricitinib, AP1066, WHI-P154, Gandotinib);
n. NF-κB-inducing kinase (NIK) inhibitors.

Contemplated herein is an HDAC inhibitor (e.g., of formula (I) or (II) combined with a signal transduction pathway inhibitor, for example, the signal transduction pathway inhibitor Gefitinib.

Also contemplated herein is a combination of a disclosed HDAC inhibitor (e.g., compound of formula (I) or (II)), with a tumor immunotherapeutic. A tumour immunotherapeutic may also be referred to as an immunomodulatory (IMiD) agent. In some embodiments, the tumour immunotherapeutic is selected from the list below:
Small molecules
   a. PI3K inhibitors (e.g. those listed in WO 2011/021038 and WO 2015/121657);
   b. Indoleamine-2,3-dioxygenase (IDO) inhibitors (e.g. NLG919, INCB024360, Indoximod);
   c. Immunomodulators (IMiDs) (e.g. Lenalidomide, Pomalidomide, Thalidomide);
Biological agents
   a. Anti-PD-1 agents: (e.g. Pembrolizumab, Nivolumab, Pidilizumab, AMP-224);
   b. Anti-PD-L1 agents (e.g. MSB0010718C, Atezolizumab, MED14736, MPDL3280A);
   c. CTLA-4-targeted agents (e.g. lpilimumab).

In an embodiment, a disclosed HDAC inhibitor such as compound of formula (I) or II may be combined with Agents inhibiting the BCL2 family of proteins (such as BCL-2, BCL-xL, BCL-w). Examples of such agents include ABT-737, ABT-263, Obatoclax, Venetoclax, Sabutoclax, AT101, HA14-1, BAM7.

Preferably, when e.g., compound of formula (I) is combined with a tumour immunotherapeutic, the tumour immunotherapeutic is Lenalidomide of Pomalidomide.

Also contemplated herein are disclosed HDAC inhibitors in combination with an agent inhibiting Mcl-1 (e.g. UMI-77).

A disclosed HDAC inhibitor such as a compound of formula (I) or (II) may be combined with Proteasome Inhibitors (e.g. Carfilzomib, Bortezomib, MG-132, MLN9708, Ixazomib, ONX-0914, Oprozomib, PI-1840, CEP-18770, Celastrol). Preferably, when a a disclosed HDAC inhibitor is combined with a proteasome inhibitor, the proteasome inhibitor is Bortezomib or Carfilzomib.

A disclosed HDAC inhibitor such as a compound of formula (I) or (II) may be combined with Poly (ADP-ribose) poymerase (PARP) Inhibitors (e.g. Olaparib, Veliparib, Rucaparib, lnipararib, Talazoparib, G007-LK, NU1025, AG-14361, INO-1001, UPF-1069, AZD-2461, PJ34, ME0328, A-966492).

A disclosed HDAC inhibitor such as a compound of formula (I) or (II) may be combined with Aromatase inhibitors (e.g. Letrozole, Anastrazole).

A disclosed HDAC inhibitor such as a compound of formula (I) or (II) may be combined with Conventional cytotoxic agents including: platinum complexes, e.g. cisplatin and carboplatin; mitoxantrone; vinca alkaloids e.g. vincristine and vinblastine; anthracycline antibiotics, e.g. daunorubicin and doxorubicin; alkylating agents, e.g. chlorambucil and melphalan; taxanes e.g. paclitaxel; antifolates, e.g. methotrexate and tomudex; epipodophyllotoxins, e.g. etoposide; camptothecins, e.g. irinotecan and its active metabolite SN38; DNA methylation inhibitors, e.g. the DNA methylation inhibitors disclosed in WO02/085400.

A disclosed HDAC inhibitor such as a compound of formula (I) or (II) may be combined with a miscellaneous agent selected from Abiraterone, ARN-509, MYC inhibitors.

### General Description—Compositions (Combinations)

A pharmaceutical composition of the invention comprises a compound/combination as defined above, and a pharmaceutically acceptable carrier or diluent. A pharmaceutical composition of the invention typically contains up to 85 wt % of a compound of the invention. More typically, it contains up to 50 wt % of a compound of the invention. Preferred pharmaceutical compositions are sterile and pyrogen-free. Further, the pharmaceutical compositions provided by the invention typically contain a compound of the invention which is a substantially pure optical isomer. Preferably, the pharmaceutical composition comprises a pharmaceutically acceptable salt form of a compound of the invention. For example, contemplated herein is a pharmaceutically acceptable composition comprising a disclosed compound and a pharmaceutically acceptable excipient.

As used herein, a pharmaceutically acceptable salt is a salt with a pharmaceutically acceptable acid or base. Pharmaceutically acceptable acids include both inorganic acids such as hydrochloric, sulfuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, ethanedisulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

For the avoidance of doubt, the present invention also embraces pro-drugs which react in vivo to give a compound of the present invention.

The compounds of Formula (I) of the invention may be prepared by synthetic routes that will be apparent to those skilled in the art, e.g. based on the Examples.

The compounds of Formula (I) of the invention and compositions comprising them may be administered in a variety of dosage forms. In one embodiment, a pharmaceutical composition comprising a compound of the invention may be formulated in a format suitable for oral, rectal, parenteral, intranasal or transdermal administration or administration by inhalation or by suppository. Typical routes of administration are parenteral, intranasal or transdermal administration or administration by inhalation.

The compounds of Formula (I) of the invention and the compositions of the invention can be administered orally, for example as tablets, troches, lozenges, aqueous or oily suspensions, dispersible powders or granules. Preferred pharmaceutical compositions of the invention are compositions suitable for oral administration, for example tablets and capsules.

The compounds of Formula (I) of the invention and the compositions of the invention may also be administered parenterally, whether subcutaneously, intravenously, intramuscularly, intrasternally, transdermally or by infusion techniques. The compounds may also be administered as suppositories.

The compounds of Formula (I) of the invention and the compositions may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed. A further advantage may be to treat diseases of the pulmonary system, such that delivering drugs by inhalation delivers them to the proximity of the cells which are required to be treated.

The present invention also provides an inhalation device containing such a pharmaceutical composition. Typically said device is a metered dose inhaler (MDI), which contains a pharmaceutically acceptable chemical propellant to push the medication out of the inhaler.

The compositions of the invention may also be administered by intranasal administration. The nasal cavity's highly permeable tissue is very receptive to medication and absorbs it quickly and efficiently, more so than drugs in tablet form. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing such a pharmaceutical composition.

The compositions of the invention may also be administered by transdermal administration. The present invention therefore also provides a transdermal patch containing a compound of the invention.

The compositions of the invention may also be administered by sublingual administration. The present invention therefore also provides a sub-lingual tablet comprising a compound of the invention.

A compositions of the invention may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compositions or methods of the present invention can be used in both the treatment and prevention of cancer and can be used in a monotherapy or in a combination therapy. When used in a combination therapy, the compounds of the present invention are typically used together with small chemical compounds such as platinum complexes, anti-metabolites, DNA topoisomerase inhibitors, radiation, antibody-based therapies (for example herceptin and rituximab), anti-cancer vaccination, gene therapy, cellular therapies, hormone therapies or cytokine therapy.

HDAC is believed to contribute to the pathology and/or symptomology of several different diseases such that reduction of the activity of HDAC in a subject through inhibition of HDAC may be used to therapeutically address these disease states. Examples of various diseases that may be treated using the HDAC inhibitors of the present invention are described herein.

One set of indications that the HDAC inhibitors in contemplated combinations of the present invention may be used to treat is those involving undesirable or uncontrolled cell proliferation. Such indications include benign tumours, various types of cancers such as primary tumours and tumour metastasis, restenosis (e.g. coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularised, and proliferative responses associated with organ transplants. More specific indications for HDAC inhibitors include, but are not limited to prostate cancer, lung cancer, acute leukaemia, multiple myeloma, bladder carcinoma, renal carcinoma, breast carcinoma, colorectal carcinoma, neuroblastoma and melanoma.

In one embodiment, a method is provided for treating diseases associated with undesired and uncontrolled cell proliferation. The method comprises administering to a subject suffering from uncontrolled cell proliferation a therapeutically effective amount of a HDAC inhibitor according to the present invention, such that said uncontrolled cell proliferation is reduced, while an additional therapeutic agent is administered that may ameliorate another aspect of the disorder being treated, or may also treat uncontrolled cel proliferation The particular dosage of the HDAC inhibitor to be used will depend on the severity of the disease state, the route of administration, and related factors that can be determined by the attending physician. Generally, acceptable and effective daily doses are amounts sufficient to effectively slow or eliminate uncontrolled cel proliferation.

Compositions according to the present invention may also be used in conjunction with other agents to inhibit undesirable and uncontrolled cell proliferation. Examples of other anti-cell proliferation agents that may be used in conjunction with the HDAC inhibitors of the present invention include, but are not limited to, retinoid acid and derivatives thereof, 2-methoxyestradiol, Angiostatin™ protein, Endostatin™ protein, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((1-azetidine-2-carboxylic acid (LACA), cishydroxyproline, d,l-3,4-dehydroproline, thiaproline), beta-aminopropionitrile fumarate, 4-propyl-5-(4-pyridinyl)-2(3H)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide; angiostatic steroid, carboxyaminoimidazole; metalloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

Generally, cells in benign tumours retain their differentiated features and do not divide in a completely uncontrolled manner. A benign tumour is usually localised and nonmetastatic. Specific types of benign tumours that can be treated using HDAC inhibitors of the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In the case of malignant tumours, cells become undifferentiated, do not respond to the body's growth control signals, and multiply in an uncontrolled manner. Malignant tumours are invasive and capable of spreading to distant sites (metastasis). Malignant tumours are generally divided into two categories: primary and secondary. Primary tumours arise directly from the tissue in which they are found. Secondary tumours, or metastases, are tumours that originated elsewhere in the body but have now spread to distant organs. Common routes for metastasis are direct growth into adjacent structures, spread through the vascular or lymphatic systems, and tracking along tissue planes and body spaces (peritoneal fluid, cerebrospinal fluid, etc.).

Specific types of cancers or malignant tumours, either primary or secondary, that can be treated using the HDAC inhibitors e.g., in combination with contemplated other therapeutics as disclosed herein and include, but are not limited to, leukaemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cel carcinoma of both ulcerating and papillary type, metastatic skin carcinoma, osteo sarcoma, Ewing's sarcoma, veticulum cell sarcoma, myeloma, giant cel tumour, small-cell lung tumour, gallstones, islet cell tumour, primary brain tumour, acute and chronic lymphocytic and granulocytic tumours, hairy-cell tumour, adenoma, hyperplasia, medullary carcinoma, pheochromocytoma, mucosal neuromas, intestinal gangloneuromas, hyperplastic comeal nerve tumour, marfanoid habitus tumour, Wilms' tumour, seminoma, ovarian tumour, leiomyomater tumour, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cel tumour, polycythermia vera, adenocarcinoma, glioblastoma multiforme, leukemias, lymphomas, malignant melanomas, epidermoid carcinomas, and other carcinomas and sarcomas.

The HDAC inhibitors, e.g., in combination with contemplated other therapeutics as disclosed herein may also be used to treat abnormal cell proliferation due to insults to body tissue during surgery. These insults may arise as a result of a variety of surgical procedures such as joint surgery, bowel surgery, and cheloid scarring. Diseases that produce fibrotic tissue that may be treated using the HDAC inhibitors of the present invention include emphysema. Repetitive motion disorders that may be treated using the present invention include carpal tunnel syndrome. An example of a cell proliferative disorder that may be treated using the invention is a bone tumour.

Proliferative responses associated with organ transplantation that may be treated using HDAC inhibitors of the invention include proliferative responses contributing to potential organ rejections or associated complications. Specifically, these proliferative responses may occur during transplantation of the heart, lung, liver, kidney, and other body organs or organ systems.

Abnormal angiogenesis that may be treated using this invention include those abnormal angiogenesis accompanying rheumatoid arthritis, ischemic-reperfusion related brain edema and injury, cortical ischemia, ovarian hyperplasia and hypervascularity, polycystic ovary syndrome, endometriosis, psoriasis, diabetic retinopathy, and other ocular angiogenic diseases such as retinopathy of prematurity (retrolental fibroplastic), macular degeneration, corneal graft rejection, neuroscular glaucoma and Oster Webber syndrome.

Examples of diseases associated with uncontrolled angiogenesis that may be treated according to the present invention include, but are not limited to retinal/choroidal neovascularization and corneal neovascularization. Examples of diseases which include some component of retinal/choroidal neovascularization include, but are not limited to, Best's diseases, myopia, optic pits, Stargart's diseases, Paget's disease, vein occlusion, artery occlusion, sickle cell anemia, sarcoid, syphilis, pseudoxanthoma elasticum carotid apo structive diseases, chronic uveitis/vitritis, mycobacterial infections, Lyme's disease, systemic lupus erythematosus, retinopathy of prematurity, Eale's disease, diabetic retinopathy, macular degeneration, Bechet's diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

Chronic inflammatory diseases associated with uncontrolled angiogenesis may also be treated using HDAC inhibitors of the present invention. Chronic inflammation depends on continuous formation of capillary sprouts to maintain an influx of inflammatory cells. The influx and presence of the inflammatory cells produce granulomas and thus maintains the chronic inflammatory state. Inhibition of angiogenesis using a HDAC inhibitor alone or in conjunction with other anti-inflammatory agents may prevent the formation of the granulosmas and thus alleviate the disease. Examples of chronic inflammatory diseases include, but are not limited to, inflammatory bowel diseases such as Crohn's disease and ulcerative colitis, psoriasis, sarcoidosis, and rheumatoid arthritis.

Inflammatory bowel diseases such as Crohn's disease and ulcerative colitis are characterised by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhoea associated with abdominal pain, fever, anorexia, weight loss and abdominal swelling. Ulcerative colitis is also a chronic, nonspecific, inflammatory and ulcerative disease arising in the colonic mucosa and is characterised by the presence of bloody diarrhoea. These inflammatory bowel diseases are generally caused by chronic granulomatous inflammation throughout the gastrointestinal tract, involving new capillary sprouts surrounded by a cylinder of inflammatory cells. Inhibition of angiogenesis by these inhibitors should inhibit the formation of the sprouts and prevent the formation of granulomas. Inflammatory bowel diseases also exhibit extra intestinal manifestations, such as skin lesions. Such lesions are characterised by inflammation and angiogenesis and can occur at many sites other the gastrointestinal tract. Inhibition of angiogenesis by HDAC inhibitors according to the present invention can reduce the influx of inflammatory cells and prevent lesion formation.

Sarcoidosis, another chronic inflammatory disease, is characterised as a multisystem granulomatous disorder. The granulomas of this disease can form anywhere in the body. Thus, the symptoms depend on the site of the granulomas and whether the disease is active. The granulomas are created by the angiogenic capillary sprouts providing a constant supply of inflammatory cells. By using HDAC inhibitors according to the present invention to inhibit angiogenesis, such granulomas formation can be inhibited. Psoriasis, also a chronic and recurrent inflammatory disease, is characterised by papules and plaques of various sizes. Treatment using these inhibitors alone or in conjunction with other anti-inflammatory agents should prevent the formation of new blood vessels necessary to maintain the characteristic lesions and provide the patient relief from the symptoms.

Rheumatoid arthritis (RA) is also a chronic inflammatory disease characterised by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using HDAC inhibitors according to the present invention alone or in conjunction with other anti-RA agents may prevent the formation of new blood vessels necessary to maintain the chronic inflammation.

The compositions of the present invention can further be used in the treatment of cardiac/vasculature diseases such as hypertrophy, hypertension, myocardial infarction, reperfusion, ischaemic heart disease, angina, arrhythmias, hypercholesterolemia, atherosclerosis and stroke. The compounds can further be used to treat neurodegenerative disorders/CNS disorders such as acute and chronic neurological diseases, including stroke, Huntington's disease, Amyotrophic Lateral Sclerosis and Alzheimer's disease.

The compositions of the present invention can also be used as antimicrobial agents, for example antibacterial agents. The invention therefore also provides a compound for use in the treatment of a bacterial infection. The compounds of the present invention can be used as anti-infectious compounds against viral, bacterial, fungal and parasitic infections. The invention therefore also provides a compound for use in the treatment of a viral infection (as an antiviral agent), a fungal infection (as an antifungal agent) or a parasitic infection (as an antiparasitic agent). Examples of infections include protozoal parasitic infections (including plasmodium, Cryptosporidium parvum, Toxoplasma gondii, Sarcocystis neurona and Eimeria sp.)

The compositions of the present invention are particularly suitable for the treatment of undesirable or uncontrolled cell proliferation, preferably for the treatment of benign tumours/hyperplasias and malignant tumours, more preferably for the treatment of malignant tumours and most preferably for the treatment of chronic lymphocytic leukaemia (CLL), breast cancer, prostate cancer, ovarian cancer, mesothelioma, T-cell lymphoma.

It is preferred that the compounds of the invention are used in the treatment of solid tumours and haematological tumours.

In a preferred embodiment of the invention, the compositions of the invention are used to alleviate cancer, cardiac hypertrophy, chronic heart failure, an inflammatory condition, a cardiovascular disease, a haemoglobinopathy, a thalassemia, a sickle cell disease, a CNS disorder, an autoimmune disease, organ transplant rejection, diabetes, osteoporosis, MDS, benign prostatic hyperplasia, oral leukoplakia, a genentically related metabolic disorder, an infection, Rubens-Taybi, fragile X syndrome, or alpha-1 antitrypsin deficiency, or to accelerate wound healing, to protect hair follicles or as an immunosuppressant.

Typically, said inflammatory condition is a skin inflammatory condition (for example psoriasis, acne and eczema), asthma, chronic obstructive pulmonary disease (COPD), rheumatoid arthritis (RA), inflammatory bowel disease (IBD), Crohn's disease or colitis.

Typically, said cancer is chronic lymphocytic leukaemia, breast cancer, prostate cancer, ovarian cancer, mesothelioma or T-cell lymphoma.

Typically, said cardiovascular disease is hypertension, myocardial infarction (MI), ischemic heart disease (IHD) (reperfusion), angina pectoris, arrhythmia, hypercholesterolemia, hyperlipidaemia, atherosclerosis, stroke, myocarditis, congestive heart failure, primary and secondary i.e. dilated (congestive) cardiomyopathy, hypertrophic cardiomyopathy, restrictive cardiomyopathy, peripheral vascular disease, tachycardia, high blood pressure or thrombosis.

Typically, said genentically related metabolic disorder is cystic fibrosis (CF), peroxisome biogenesis disorder or adrenoleukodystrophy.

Typically, the compounds of the invention are used as an immunosuppressant following organ transplant

Typically, said infection is a viral, bacterial, fungal or parasitic infection, in particular an infection by S aureus, P acne, candida or aspergillus.

Typically, said CNS disorder is Huntingdon's disease, Alzheimer's disease, multiple sclerosis or amyotrophic lateral sclerosis.

In this embodiment, the compositions of the invention may be used to alleviate cancer, cardiac hypertrophy, chronic heart failure, an inflammatory condition, a cardiovascular disease, a haemoglobinopathy, a thalassemia, a sickle cel disease, a CNS disorder, an autoimmune disease, diabetes or osteoporosis, or are used as an immunosuppressant.

The compositions of the invention may also be used to alleviate chronic lymphocytic leukaemia (CLL), breast cancer, prostate cancer, ovarian cancer, mesothelioma, T-cell lymphoma, cardiac hypertrophy, chronic heart failure or a skin inflammatory condition, in particular psoriasis, acne or eczema.

The compositions of the present invention can be used in the treatment of animals, preferably in the treatment of mammals and more preferably in the treatment of humans.

The compositions of the invention may, where appropriate, be used prophylactically to reduce the incidence of such conditions.

In use, a therapeutically effective amount of a compound of the invention is administered to a patient A typical dose is from about 0.001 to 50 mg per kg of body weight, according to the activity of the specific compound, the age, weight and conditions of the subject to be treated, the type and severity of the disease and the frequency and route of administration.

Where a kit and/or a method of the invention provides for the administration of more than one drug, they can be administered simultaneous, sequentially or separately. It is not necessary that they are packed together (but this is one embodiment of the invention). It is also not necessary that they are administered at the same time or that they are in the same dosage form. As used herein, “separate” administration means that the drugs are administered as part of the same overall dosage regimen (which could comprise a number of days), but preferably on the same day. As used herein “simultaneously” means that the drugs are to be taken together or formulated as a single composition. As used herein, “sequentially” means that the drugs are administered at about the same time, and preferably within about 1 hour of each other.

In some embodiments, a disclosed HDAC inhibitor may be administered at certain dosages (e.g., lower dosages than monotherapy) but may be therapeutically effective when combined with certain anti-tumour compounds such as those disclosed herein. For example, the combination of the HDAC inhibitor of Formula I and certain anti-tumour compounds disclosed herein may achieve a synergistic effect in the treatment of the subject in need thereof, wherein the combination is administered at dosages that would not be effective when one or both of the compounds are administered alone, but which amounts are effective in combination.

### EXAMPLES

General Methods

i. General Procedure for Synthesis of Secondary Amines

Method A (Using BINAP):

4,6-Dimethylpyridin-2-amine (200 mg, 1.63 mmol), 2-bromo-5-fluoropyridine (317 mg, 1.8 mmol), potassium tert-butoxide (236 mg, 2.45 mmol) and (t)-BINAP (40 mg, 0.06 mmol) were stirred in toluene (4 mL) and degassed using Ar(g) for 30 min. Pd₂(dba)₃ (45 mg, 0.049 mmol) was then added and the reaction mixture stirred for 12 h at 90° C. under Ar(g). The reaction was monitored by TLC. Following complete consumption of starting material, the reaction mixture was diluted with CH₂Cl₂ (20 mL) and silica was added. The solvent was removed in vacuo and the resulting dry loaded material was purified by silica gel column chromatography with hexane/EtOAc (4:1-1:1) to provide N-(5-fluoropyridin-2-yl)-4,6-dimethylpyridin-2-amine.

Method B (Using SPhos):

2-Bromopyridine (200 mg, 1.26 mmol), 5-methylpyridin-2-amine (150 mg, 1.38 mmol), potassium tert-butoxide (182 mg, 1.89 mmol) and 2-dicyclohexylphosphino-2′,6′-dimethoxybiphenyl (SPhos) (20 mg, 0.05 mmol) were stirred in toluene (4 mL) and the reaction mixture was degassed using Ar(g) for 30 min. Pd₂(dba)₃ (34 mg, 0.037 mmol) was then added, and the reaction mixture was stirred for 12 h at 90° C. under Ar(g). The reaction was monitored by TLC. Following complete consumption of the starting material, the reaction mixture was diluted with CH₂Cl₂ (20 mL) and silica was added. The solvent was removed in vacuo, and the resulting dry loaded material was purified by silica gel column chromatography with hexane/EtOAc, (4:1-1:1) to provide N-(pyridin-2-yl)-5-methylpyridin-2-amine.

### a) 3-Methoxy-N-(5-methylpyridin-2-yl)pyridin-2-amine

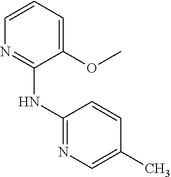 [SMILES: COc1cccnc1Nc1ccc(C)cn1]

Synthesised according to the general procedure Method B (Using SPhos).

¹H NMR (400 MHz, Chloroform-d), δ_{H} ppm: 8.44 (d, J=8.6 Hz, 1H), 8.02-8.13 (m, 1H), 7.73-7.93 (m, 2H), 7.48 (dd, J=8.6, 2.3 Hz, 1H), 6.99 (dd, J=7.8, 1.5 Hz, 1H), 6.83-6.71 (m, 1H), 3.89 (s, 3H), 2.27 (s, 3H).

### b) 5-Methoxy-N-5-methylpyridin-2-yl)pyridin-2-amine

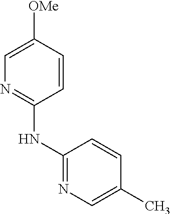 [SMILES: COc1ccc(Nc2ccc(C)cn2)nc1]

Synthesised according to the general procedure Method B (Using SPhos).

¹H NMR (400 MHz, Chloroform-d), δ_{H} ppm: 8.04 (d, J=2.5 Hz, 1H), 7.95 (d, J=3.0 Hz, 1H), 7.50 (d, J=9.0 Hz, 1H), 7.40 (dd, J=8.4, 2.6 Hz, 1H), 7.31 (d, J=8.4 Hz, 1H), 7.22 (dd, J=9.0, 3.1 Hz, 1H), 3.87 (m, 3H), 2.25 (s, 3H).

### c) 3-Methoxy-N-(5-morpholinopyridin-2-yl)pyridin-2-amine

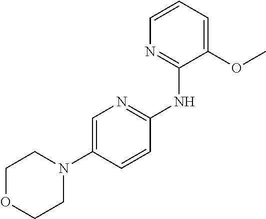 [SMILES: COc1cccnc1Nc1ccc(N2CCOCC2)cn1]

Synthesised according to the general procedure Method B (Using SPhos).

¹H NMR (400 MHz, Chloroform-d), δ_{H} ppm: 8.45 (d, J=9.1 Hz, 1H), 7.94 (d, J=3.0 Hz, 1H), 7.83 (dd, J=5.1, 1.5 Hz, 1H), 7.31 (dd, J=9.1, 3.1 Hz, 1H), 6.98 (dd, J=7.9, 1.5 Hz, 1H), 6.73 (dd, J=7.8, 5.1 Hz, 1H), 3.76-3.98 (m 7H), 3.06-3.16 (m 4H).

### d) 5-Methoxy-N-(5-morpholinopyridin-2-yl)pyridin-2-amine

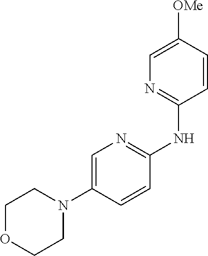 [SMILES: COc1ccc(Nc2ccc(N3CCOCC3)cn2)nc1]

Synthesised according to the general procedure Method B (Using SPhos).

¹H NMR (400 MHz, Chloroform-d), δ_{H} ppm: 7.90 (dd, J=15.8, 3.0 Hz, 2H), 7.43 (d, J=9.0 Hz, 2H), 7.19-7.30 (m, 2H), 3.87 (t, J=4.8 Hz, 4H), 3.82 (s, 3H), 3.00-3.16 (m, 4H).

### e) N-(Pyridin-2-yl)thieno[3,2-c]pyridin-4-amine

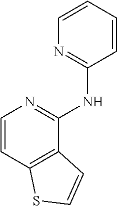 [SMILES: c1ccc(Nc2nccc3sccc23)nc1]

Synthesised according to the general procedure Method B (Using SPhos).

¹H NMR (400 MHz, Chloroform-d), δ_{H} ppm: 8.58 (d, J=8.4 Hz, 1H), 8.26 (dd, J=5.1, 2.0 Hz, 1H), 8.12 (d, J=5.7 Hz, 1H), 7.72 (ddd, J=8.8, 7.1, 1.9 Hz, 1H), 7.51 (d, J=5.9 Hz, 1H), 7.46 (d, J=5.4 Hz, 1H), 7.38 (d, J=5.7 Hz, 1H), 6.93 (ddd, J=7.1, 4.8, 1.0 Hz, 1H).

### f) 6-Methyl-N-(5-morpholinopyridin-2-yl)pyridin-2-amine

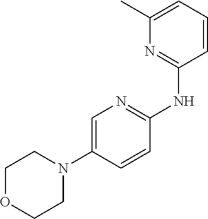 [SMILES: Cc1cccc(Nc2ccc(N3CCOCC3)cn2)n1]

Synthesised according to the general procedure Method B (Using SPhos).

¹H NMR (400 MHz, Chloroform-d), δ_{H} ppm: 7.94 (d, J=3.0 Hz, 1H), 7.40-7.59 (m, 2H), 7.24 (d, J=8.1 Hz, 2H), 6.66 (d, J=7.3 Hz, 1H), 3.80-3.96 (m, 4H), 3.01-3.17 (m, 4H), 2.45 (s, 3H).

### g) N-(6-(Trifluoromethyl)pyridin-2-yl)thieno[3,2-c]pyridin-4-amine

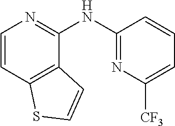 [SMILES: FC(F)(F)c1cccc(Nc2nccc3sccc23)n1]

Synthesised according to the general procedure Method A (Using BINAP).

¹H NMR (400 MHz, Chloroform-d), δ_{H} ppm: 8.82 (d, J=8.5 Hz, 1H), 8.14 (d, J=5.7 Hz, 1H), 7.83 (dd, J=18.3, 10.3 Hz, 2H), 7.51 (s, 1H), 7.44 (d, J=5.7 Hz, 1H), 7.29 (d, J=7.4 Hz, 1H).

### h) N5-(2-Methoxyethyl)-N5-methyl-N2-(4-(trifluoromethyl)pyridin-2-yl)pyridine-2,5-diamine

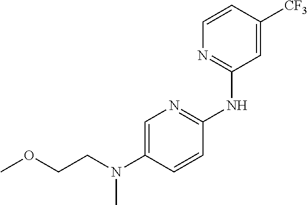 [SMILES: COCCN(C)c1ccc(Nc2cc(C(F)(F)F)ccn2)nc1]

Synthesised according to the general procedure Method A (Using BINAP).

¹H NMR (400 MHz, Chloroform-d), δ_{H} ppm: 8.32 (d, J=5.2 Hz, 1H), 7.87 (d, J=3.1 Hz, 1H), 7.70-7.78 (m, 1H), 7.29-7.37 (m, 1H), 7.15 (dd, J=9.0, 3.1 Hz, 1H), 6.88-6.98 (m, 1H), 3.54-3.59 (m, 2H), 3.48 (t, J=5.5 Hz, 2H), 3.37 (s, 3H), 2.98 (s, 3H).

### i) N5-(2-Methoxyethyl)-N2-(3-methoxypyridin-2-yl)-N5-methylpyridine-2,5-diamine

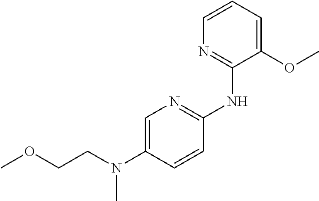

Synthesised according to the general procedure Method B (Using SPhos).

¹H NMR (400 MHz, Chloroform-d), δ_{H} ppm: 8.37 (d, J=9.1 Hz, 1H), 7.80-7.82 (m, 2H), 7.19 (dd, J=9.1, 3.1 Hz, 1H), 6.96 (dd, J=7.7, 1.5 Hz, 1H), 6.70 (dd, J=7.8, 5.1 Hz, 1H), 3.88 (s, 3H), 3.56 (t, J=5.8 Hz, 2H), 3.45 (t, J=5.8 Hz, 2H), 3.36 (s, 3H), 2.96 (s, 3H).

### j) N5-(2-methoxyethyl)-N2-(5-methoxypyridin-2-yl)-N5-methylpyridine-2,5-diamine

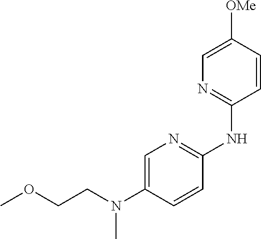 [SMILES: COCCN(C)c1ccc(Nc2ccc(OC)cn2)nc1]

Synthesised according to the general procedure Method B (Using SPhos).

¹H NMR (400 MHz, Chloroform-d), δ_{H} ppm: 7.89 (d, J=3.0 Hz, 1H), 7.74 (d, J=3.1 Hz, 1H), 7.45 (d, J=9.1 Hz, 1H), 7.37 (d, J=9.0 Hz, 1H), 7.16-7.22 (m, 2H), 3.82 (s, 3H), 3.55 (t, J=5.8 Hz, 2H), 3.43 (t, J=5.8 Hz, 2H), 3.36 (s, 3H), 2.94 (s, 3H).

iii. General Procedure for Alkylation and Hydroxamic Acid Formation

NaH (12 mg, 0.5 mmol, 2 eq) was added portion-wise to secondary amine (50 mg, 0.25 mmol, 1 eq) in DMF (2 mL) at 0° C. under Ar(g). Following addition, the reaction mixture was stirred for 20 min, then methyl-4-(bromomethyl)benzoate (57 mg, 0.25 mmol, 1 eq) was added. The reaction mixture was stirred at rt under Ar(g) for 2 h, and the reaction was monitored by TLC. Following complete consumption of the starting material, the reaction mixture was poured onto brine (25 mL), extracted with EtOAc (3×25 mL). The organic phases were combined, dried over Na₂SO₄, filtered and subsequently concentrated in vacuo. The resulting crude product was purified by silica gel column chromatography with hexane/EtOAc (19:1-3:1) to provide the desired methyl ester as a gummy, yellowish solid.

To a stirred solution of the methyl ester (70 mg, 0.20 mmol) in MeOH/CH₂Cl₂ (3:1, 4 mL) under an inert atmosphere was added 50% aq. hydroxylamine solution (2.5 mL) at ° C., and the resulting reaction mixture was stirred for 20 min. Sodium hydroxide solution (54 mg in 1 mL water, 1.35 mmol) was then added to the reaction mixture; this was following by stirring for 30 min. and the mixture was then warmed to rt and stirred for 2 h. The reaction was monitored by TLC. Following complete consumption of the starting material, the volatiles were concentrated in vacuo. The residue was acidified with acetic acid to pH˜6. The compound was extracted with CH₂Cl₂/MeOH (9:1) (3×20 mL); the combined organic extracts were concentrated in vacuo to obtain the crude product, which was purified by silica gel column chromatography (1-10% MeOH/CH₂Cl₂) to afford the desired product as gummy, yellowish solid.

### SPECIFIC EXAMPLES

### Example A

### 4-{[Bis(pyridin-2-yl)amino]methyl}-N-hydroxybenzamide

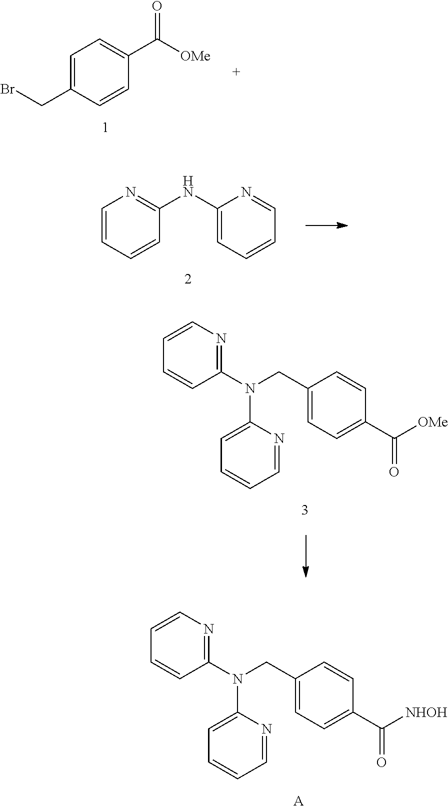

NaH (83 mg, 2.18 mmol) was added to 2,2′-dipyridylamine, (2) (373 mg, 2.18 mmol) in DMF (5 mL) at rt. After 15 min, methyl-4-(bromomethyl)benzoate (1) (500 mg, 2.18 mmol) was added, and the reaction mixture was subsequently stirred at 90° C. for 1 h under Ar(g). Once cooled to rt, the reaction mixture was poured onto brine (50 mL) and extracted with EtOAc (2×25 mL). The organic phases were combined, dried over MgSO₄, filtered and subsequently concentrated in vacuo. The resulting residue was purified by silica gel column chromatography with hexane/EtOAc (4:1) to furnish (3) as a white solid (429 mg, 62%).

LCMS (ES): found 319.9 [M+H]⁺.

A freshly prepared solution of NH₂OH in MeOH (0.4M, 20 mL) was added to 4-{[bis(pyridin-2-yl)amino]methyl}benzoate (3) (100 mg, 0.3 mmol) at 0′C followed by KOH solubilised in MeOH (0.8M, 4 mL). The reaction mixture was then stirred at rt for 18 h, was subsequently concentrated in vacuo (ca 5 mL) and poured onto water (50 mL). The basic aqueous phase was extracted initially with EtOAc (25 mL) and the phases were separated. The aqueous was then neutralised with 2N HCl and extracted again with EtOAc (25 mL). The resulting organic phase was dried over MgSO₄, filtered and subsequently concentrated in vacuo to provide Example A as a white solid (51 mg, 51%).

¹H NMR (400 MHz, Methanol-d₄), δ_{H} ppm: 8.20-8.28 (m, 2H), 7.59-7.67 (m, 4H), 7.43 (d, J=8.6 Hz, 2H), 7.17 (d, J=8.1 Hz, 2H), 6.96 (dd, J=6.6, 5.1 Hz, 2H), 5.48 (s, 2H).

LCMS (ES): found 321.1 [M+H]⁺.

### Example B

### 4-{[Bis(3-methyl-1,2,4-thiadiazol-5-yl)amino]methyl}-2-fluoro-N-hydroxybenzamide

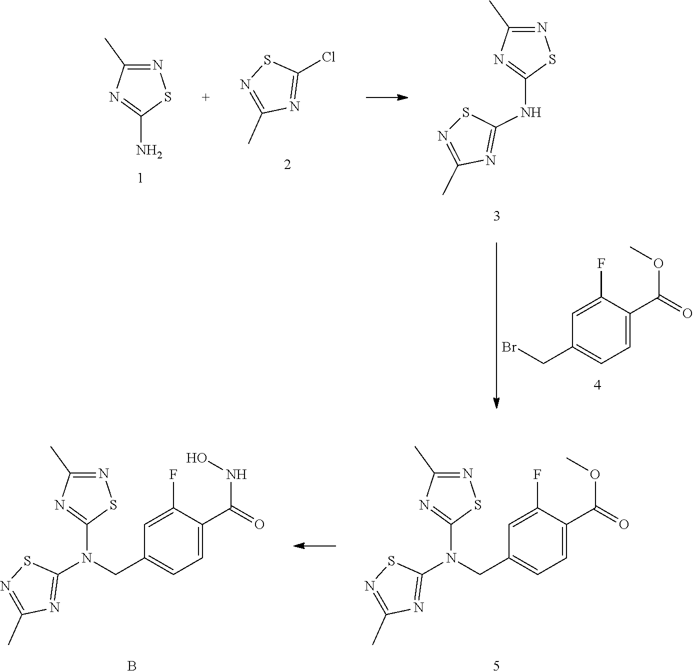

NaH (60% in oil) (50 mg) was added to a solution of 3-methyl-1,2,4-thiadiazol-5-amine (1) (115 mg, 1 mmol) in NMP (2 mL). After 10 min, 5-chloro-3-methyl-1,2,4-thiadiazole (2) (140 mg, 1.05 mmol) was added and the resultant mixture stirred at 45° C. under N₂(g). After 4 h, the reaction mixture was diluted with EtOAc and extracted with saturated bicarbonate solution (×3). Analysis indicated that all desired product was in the aqueous phase. The combined aqueous phases were concentrated to dryness; the resultant residue was slurried with MCN (2×100 mL) and filtered. The filtrate was concentrated to afford (3) as an oil/NMP solution (700 mg).

LCMS (ES): found 214.0 [M+H]⁺.

Potassium carbonate (360 mg) and methyl 4-(bromomethyl)-2-fluorobenzoate (4) (160 mg, 0.65 mmol) were added to a solution of 3-methyl-N-(3-methyl-1,2,4-thiadiazol-5-yl)-1,2,4-thiadiazol-5-amine (3) (<1 mmol) in MeCN (10 mL) and the reaction mixture was heated, under N₂(g), with stirring, at 50° C. After 2 h, the reaction mixture was cooled, diluted with EtOAc and extracted sequentially with water, saturated bicarbonate solution and saturated brine solution, and was then dried over Na₂SO₄, filtered and concentrated. Purification on silica with CH₂Cl₂/MeOH (1:0-97:3) yielded (5) as a solid (180 mg, 73%).

LCMS (ES): found 380.0 [M+H]⁺.

50% Hydroxylamine aqueous solution (2 mL) was added to a solution of methyl 4-{[bis(3-methyl-1,2,4-thiadiazol-5-yl)amino]methyl}-2-fluorobenzoate (5) (180 mg, 0.47 mmol) in MeOH (8 mL). The solution was stirred at 45° C. for 7 days, sealed in a vial. The resulting reaction mixture became heterogeneous; on cooling, a white solid was collected by filtration, washed with cold methanol and dried in vacuo to afford the title product, Example B, as solid (50 mg, 28%).

¹H NMR (400 MHz, DMSO-d₆), δ_{H} ppm: 10.90 (br. s., 1H), 9.17 (br. s., 1H), 7.51 (t, J=7.6 Hz, 1H), 7.27 (d, J=10.8 Hz, 1H), 7.16 (dd, J=7.9, 1.3 Hz, 1H), 5.57 (s, 2H), 2.50 (s, 6H).

LCMS (ES): found 381.0 [M+H]⁺.

### Example C

### 2-Fluoro-N-hydroxy-4-{[(3-methyl-1,2,4-oxadiazol-5-yl)(3-methyl-1,2,4-thiadiazol-5-yl)amino]methyl}benzamide

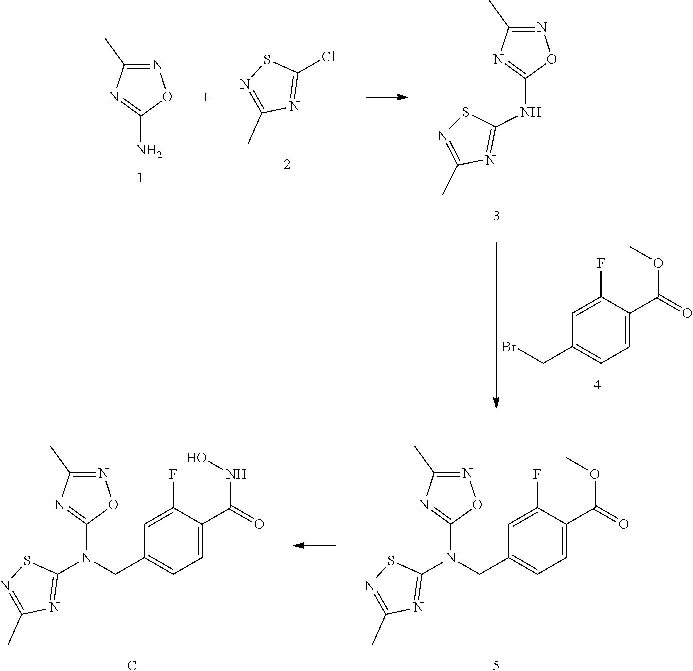

NaH (60% in oil) (50 mg) was added to a solution of 3-methyl-1,2,4-oxadiazol-5-amine (1) (100 mg, 1 mmol) in NMP (2 mL). After 10 min, 5-chloro-3-methyl-1,2,4-thiadiazole (2) (150 mg, 1.1 mmol) was added, and the resultant mixture was stirred at 45° C. under N₂(g). After 18 h, analysis by LCMS was conducted.

LCMS (ES): found 198.0 [M+H]⁺.

NaH (60% in oil) (70 mg) and methyl 4-(bromomethyl)-2-fluorobenzoate (4) (200 mg, 0.81 mmol) were added to the above reaction mixture and heating was continued at 45° C. under N₂(g). After 3 h, a further quantity of (4) (90 mg, 0.36 mmol) was added. After an additional 2 h, the reaction mixture was cooled, diluted with EtOAc, and extracted sequentially with water and saturated bicarbonate solution (×2), then dried over Na₂SO₄, filtered and concentrated. Purification by silica gel chromatography with CH₂Cl₂/MeOH (1:0-97:3) yielded (5) (350 mg, 96% over 2 steps).

LCMS (ES): found 364.0 [M+H]⁺.

50% Hydroxylamine aqueous solution (1 mL) was added to a crude solution of methyl 4-{[bis(3-methyl-1,2,4-thiadiazol-5-yl)amino]methyl}-2-fluorobenzoate (5) (350 mg, 0.96 mmol) in methanol (5 mL). The resulting solution was stirred at 45-50° C. for 5 days, sealed in a vial. The reaction mixture turned heterogeneous and, on cooling, a white solid was filtered off and the resulting filtrate was concentrated. The filtrate was purified by RP-HPLC on Xterra 10-70% MeCN/water+0.1% formic acid, to furnish Example C (30 mg, 8%).

¹H NMR (400 MHz, Methanol-d₄), δ_{H} ppm: 7.69 (t, J=7.6 Hz, 1H), 7.12-7.22 (m, 2H), 5.48 (s, 2H), 2.44 (s, 3H), 2.32 (s, 3H).

LCMS (ES): found 365.0 [M+H]⁺.

### Example D

### N-Hydroxy-4-(((3-methyl-1,2,4-oxadiazol-5-yl)(pyridin-2-yl)amino)methyl)benzamide

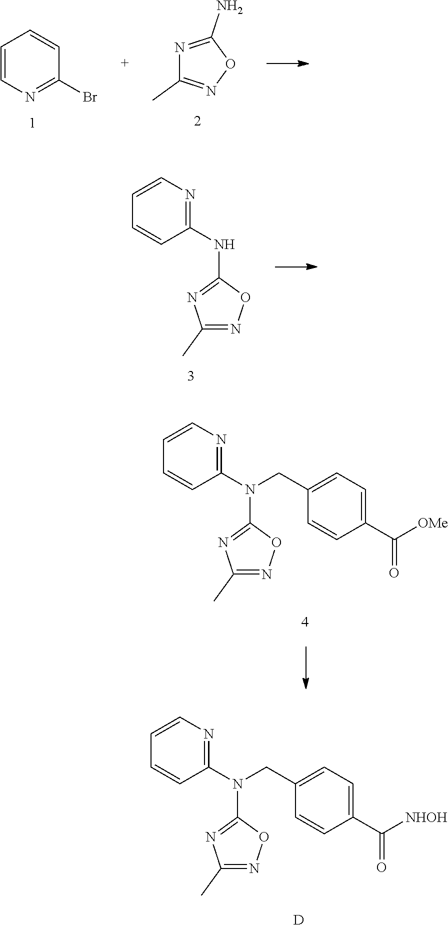

2-Bromopyridine (1) (1.0 g, 6.32 mmol), 3-methyl-1,2,4-oxadiazol-5-amine (2) (0.940 g, 9.49 mmol), Xantphos (0.366 g, 0.63 mmol), and Cs₂CO₃ (4.1 g, 12.64 mmol) were combined in dry 1,4-dioxane (15 mL). The reaction mixture was degassed with N₂(g) and placed under vacuum for 10 min. Pd₂(dba)₃ (0.28 g, 0.31 mmol) was then added to the reaction mixture, which was heated at 90° C. for 30 h. It was then poured into demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide 3-methyl-N-(pyridin-2-yl)-1,2,4-oxadiazol-5-amine (3) as a white solid (0.7 g, 63%).

LCMS (ES): Found 177.1 [M+H]⁺. NaH (60%) (52.5 mg, 1.31 mmol) was added portion-wise to 3-methyl-N-(pyridin-2-yl)-1,2,4-oxadiazol-5-amine (3) (220 mg, 1.25 mmol) in DMF (5 mL) at 5° C. under Ar(g). The reaction mixture was stirred for 20 min, then methyl 4-(bromomethyl) benzoate (372 mg, 1.62 mmol) was added, and stirring was continued at 80° C. under Ar(g) for 1 h. The reaction mixture was then poured onto demineralised water (100 mL), and extracted with EtOAc (3×50 mL). The organic phases were combined, dried over Na₂SO₄, filtered and subsequently concentrated in vacuo. The resulting residue was purified by flash chromatography with EtOAc/Hexane (1:1) to furnish methyl 4-(((3-methyl-1,2,4-oxadiazol-5-yl)(pyridin-2-yl)amino)methyl)benzoate (4) as a white solid (130 mg, 40%).

LCMS (ES): Found 325.1 [M+H]⁺.

A fresh solution of NH₂OH in MeOH was prepared: [KOH (0.91 g, 16.3 mmol) in MeOH (10 mL) was added to NH₂OH.HCl (1.12 g, 16.3 mmol) in MeOH (10 mL) at 0° C.]. The reaction mixture was stirred for 20 min at 0° C., then filtered to remove salts; it was then added to methyl 4-(((3-methyl-1,2,4-oxadiazol-5-yl)(pyridin-2-yl)amino)methyl)benzoate (4) (105.5 mg, 0.3 mmol) followed by KOH (181 mg, 3.2 mmol) solubilised in MeOH (5 mL). The reaction mixture was stirred at rt for 21 h, and then concentrated in vacuo, poured onto brine/H₂O (15 mL/35 mL), and extracted with CH₂Cl₂ (3×50 mL). The organic phases were combined, dried over Na₂SO₄, filtered and subsequently concentrated in vacuo. The resulting residue was purified by flash chromatography with MeOH/CH₂Cl₂ (10:90) to provide N-hydroxy-8-((3-methyl-1,2,4-oxadiazol-5-yl)(pyridin-2-yl)amino)octanamide, Example D, as a light yellow solid (12.2 mg, 40%).

¹H NMR (400 MHz, DMSO-d₆), δ_{H} ppm: 11.14 (br. s., 1H), 9.01 (br. s., 1H), 8.42 (dd, J=4.8, 1.1 Hz, 1H), 8.07 (d, J=8.4 Hz, 1H), 7.92 (ddd, J=8.5, 7.4, 2.0 Hz, 1H), 7.66 (d, J=8.3 Hz, 2H), 7.34 (d, J=8.3 Hz, 2H), 7.23 (ddd, J=7.3, 4.9, 0.8 Hz, 1H), 5.48 (s, 2H), 2.23 (s, 3H).

LCMS (ES): Found 326.1 [M+H]⁺.

### Example E

### N-Hydroxy-4-(((1-methyl-1H-pyrazol-3-yl)(pyridin-2-yl)amino)methyl)benzamide

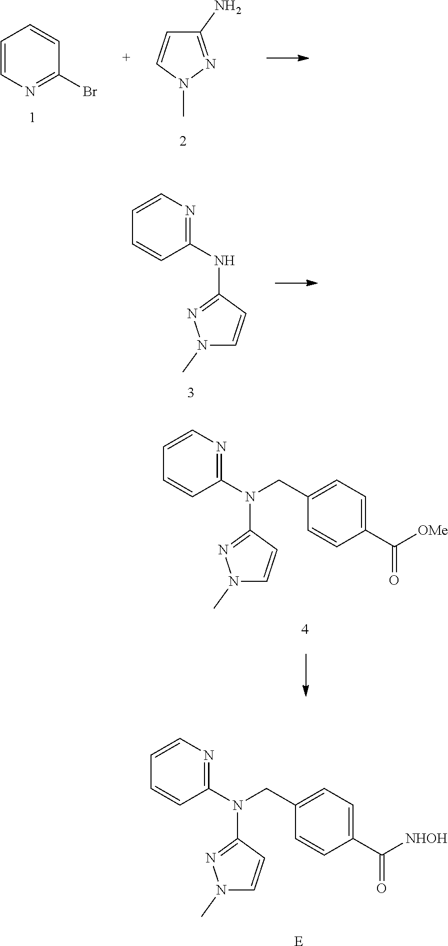

2-Bromopyridine (1) (1.0 g, 6.3 mmol), 1-methyl-1H-pyrazol-3-amine (2) (0.79 g, 8.2 mmol), Xantphos (0.37 g, 0.63 mmol), and Cs₂CO₃ (4.1 g, 12.6 mmol) were combined in dry 1,4-dioxane (15 mL). The reaction mixture was then degassed with N₂(g), and placed under vacuum for 10 min. Pd₂(dba)₃ (0.29 g, 0.31 mmol) was added and the resulting reaction mixture was heated at 90° C. for 30 h. It was then poured onto demineralised water (200 mL), and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(1-methyl-1H-pyrazol-3-yl)pyridin-2-amine (3) as a yellow solid (0.75 g, 68%).

LCMS (ES): Found 175.2 [M+H]⁺.

NaH (60%) (60.4 mg, 1.5 mmol) was added portion-wise to N-(1-methyl-1H-pyrazol-3-yl)pyridin-2-amine (3) (250 mg, 1.4 mmol) in DMF (8 mL) at 5° C. under Ar(g). The reaction mixture was stirred for 20 min, then methyl 4-(bromomethyl)benzoate (428 mg, 1.8 mmol) was added, and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((1-methyl-1H-pyrazol-3-yl)(pyridin-2-yl)amino)methyl)benzoate (4) as a light yellow solid (440 mg, 82%).

LCMS (ES): Found 323.1 [M+H]⁺.

A fresh solution of NH₂OH in MeOH was prepared: [KOH (3.83 g, 68.3 mmol) in MeOH (20 mL) was added to NH₂OH.HCl (4.74 g, 68.3 mmol) in MeOH (20 mL) at 0° C.]. The reaction mixture was stirred for 20 min at 0° C., then filtered to remove salts; it was then added to 4-(((1-methyl-1H-pyrazol-3-yl)(pyridin-2-yl)amino)methyl)benzoate (4) (440 mg, 1.3 mmol) followed by KOH (766 mg, 13.0 mmol) solubilised in MeOH (10 mL). The reaction mixture was stirred at rt for 21 h, then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-(((1-methyl-1H-pyrazol-3-yl)(pyridin-2-yl)amino)methyl)benzamide, Example E, as a light brown liquid (50 mg, 11%).

¹H NMR (400 MHz, Methanol-d₄), δ_{H} ppm: 8.09 (ddd, J=5.0, 1.9, 0.8 Hz, 1H), 7.64 (d, J=8.3 Hz, 2H), 7.52 (d, J=2.3 Hz, 1H), 7.49 (ddd, J=8.7, 7.0, 1.9 Hz, 1H), 7.40 (d, J=8.4 Hz, 2H), 6.91 (d, J=8.6 Hz, 1H), 6.73 (ddd, J=7.1, 5.1, 0.7 Hz, 1H), 6.10 (d, J=2.4 Hz, 1H), 5.26 (s, 2H), 3.81 (s, 3H).

LCMS (ES): Found 324.4 [M+H]⁺.

### Example F

### N-Hydroxy-4-((pyridin-2-yl(1,3,4-thiadiazol-2-yl)amino)methyl)benzamide

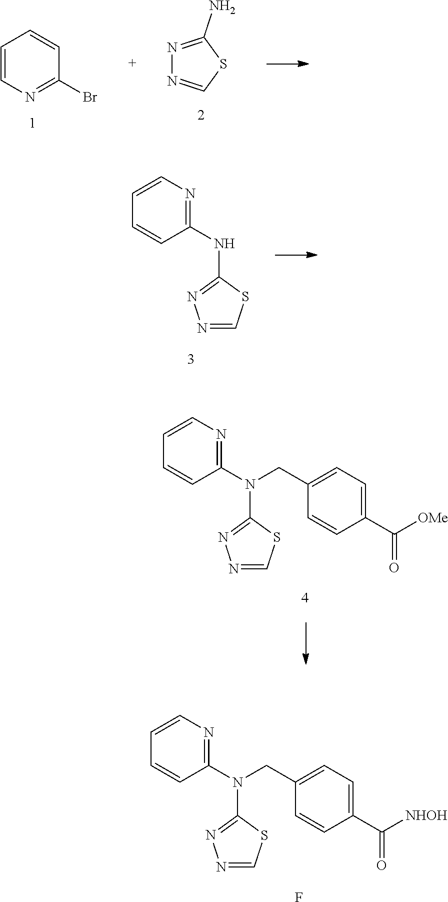

2-Bromopyridine (1) (1.0 g, 6.3 mmol), 1,3,4-thiadiazol-2-amine (2) (0.64 g, 6.3 mmol), Xantphos (0.37 g, 0.63 mmol), and Cs₂CO₃ (3.1 g, 9.4 mmol) were combined in dry 1,4-dioxane (15 mL). The reaction mixture was degassed with N₂(g) and placed under vacuum for 10 min. Pd₂(dba)₃ (0.29 g, 0.31 mmol) was then added and the resulting reaction mixture was then heated at 90° C. for 30 h. It was then poured onto demineralised water (200 mL), and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(pyridin-2-yl)-1, 3, 4-thiadiazol-2-amine (3) as a yellow solid (0.33 g, 30%).

LCMS (ES): Found 179.0 [M+H]⁺.

NaH (60%) (53 mg, 1.3 mmol) was added portion-wise to N-(pyridin-2-yl)-1,3,4-thiadiazol-2-amine (3) (225 mg, 1.26 mmol) in DMF (8 mL) at 5° C. under Ar(g). The reaction mixture was stirred for 20 min, then methyl 4-(bromomethyl)benzoate (336 mg, 1.6 mmol) was added, and stirring was continued at 70° C. under Ar(g) for 1 h in the dark. The reaction mixture was then poured onto demineralised water (100 mL), and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((pyridin-2-yl(1,3,4-thiadiazol-2-yl)amino)methyl)benzoate (4) as a light yellow solid (118 mg, 33%).

LCMS (ES): Found 327.3 [M+H]⁺.

A fresh solution of NH₂OH in MeOH was prepared: [KOH (1.01 g, 18.1 mmol) in MeOH (20 mL) was added to NH₂OH.HCl (1.26 g, 18.1 mmol) in MeOH (20 mL) at 0° C.]. The reaction mixture was stirred for 20 min at 0° C., then filtered to remove salts; it was then added to methyl 4-((pyridin-2-yl(1,3,4-thiadiazol-2-yl)amino)methyl)benzoate (4) (118 mg, 0.36 mmol) followed by KOH (203 mg, 3.6 mmol) solubilised in MeOH (10 mL). The reaction mixture was stirred at rt for 21 h, then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-((pyridin-2-yl(1,3,4-thiadiazol-2-yl)amino)methyl)benzamide, Example F, as a light brown liquid (15 mg, 13%).

¹H NMR (400 MHz, Methanol-d₄), δ_{H} ppm: 8.96 (s, 1H), 8.44 (dd, J=5.0, 1.1 Hz, 1H), 7.72-7.78 (m, 1H), 7.69 (d, J=8.2 Hz, 2H), 7.33 (d, J=8.2 Hz, 2H), 7.06-7.11 (m, 2H), 5.79 (s, 2H).

LCMS (ES): Found 328.1 [M+H]⁺.

### Example G

### N-Hydroxy-4-((pyrazin-2-yl(pyridin-2-yl)amino)methyl)benzamide

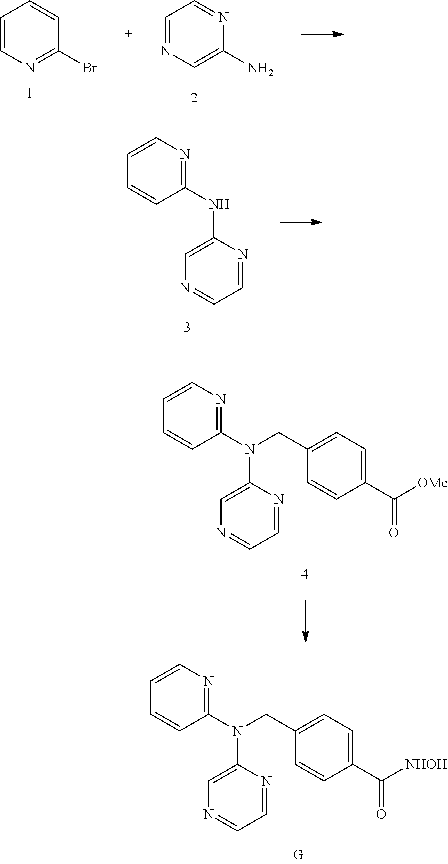

2-Bromopyridine (1) (1.0 g, 6.3 mmol), pyrazin-2-amine (2) (0.67 g, 6.9 mmol), BINAP (0.12 g, 0.18 mmol), t-BuOK (0.99 g, 8.8 mmol) were combined in dry toluene (15 mL). The reaction mixture was degassed with N₂(g) and placed under vacuum for 10 min. Pd₂(dba)₃ (0.11 g, 0.12 mmol) was added, and the mixture heated at 90° C. for 3 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(pyridin-2-yl)pyrazin-2-amine (3) as a yellow solid (0.9 g, 83%).

LCMS (ES): Found 173.1 [M+H]⁺.

NaH (60%) (61 mg, 1.52 mmol) was added portion-wise to N-(pyridin-2-yl)pyrazin-2-amine (3) (250 mg, 1.45 mmol) in DMF (10 mL) at 5° C. under Ar(g). The reaction mixture was stirred for 20 min, then methyl 4-(bromomethyl)benzoate (432 mg, 1.88 mmol) was added, and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((pyrazin-2-yl(pyridin-2-yl)amino)methyl)benzoate (4) as a light yellow solid (380 mg, 81%).

LCMS (ES): Found 321.3 [M+H]⁺.

A fresh solution of NH₂OH in MeOH was prepared: [KOH (3.33 g, 59.0 mmol) in MeOH (20 mL) was added to NH₂OH.HCl (4.1 g, 59.0 mmol) in MeOH (20 mL) at 0° C.]. The reaction mixture was stirred for 20 min at 0° C., then filtered to remove salts; it was then added to methyl 4-((pyrazin-2-yl(pyridin-2-yl)amino)methyl)benzoate (4) (380 mg, 1.1 mmol) followed by KOH (666 mg, 11.8 mmol) solubilised in MeOH (10 mL). The reaction mixture was stirred at rt for 21 h, then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-((pyrazin-2-yl(pyridin-2-yl)amino)methyl)benzamide, Example G, as a light cream solid (20 mg, 5%).

¹H NMR (400 MHz, DMSO-d₆), δ_{H} ppm: 11.10 (br. s., 1H), 8.99 (br. s., 1H), 8.65 (d, J=1.4 Hz, 1H), 8.32 (ddd, J=4.9, 1.9, 0.8 Hz, 1H), 8.27 (dd, J=2.7, 1.5 Hz, 1H), 8.10 (d, J=2.6 Hz, 1H), 7.74 (ddd, J=8.4, 7.3, 2.0 Hz, 1H), 7.64 (d, J=8.3 Hz, 2H), 7.36 (d, J=8.2 Hz, 2H), 7.33 (d, J=8.4 Hz, 1H), 7.06 (ddd, J=7.3, 4.9, 0.8 Hz, 1H), 5.45 (s, 2H).

LCMS (ES): Found 322.3 [M+H]⁺.

### Example H

### N-Hydroxy-4-(((5-methyl-1,3,4-thiadiazol-2-yl)pyridin-2-yl)amino)methyl)benzamide

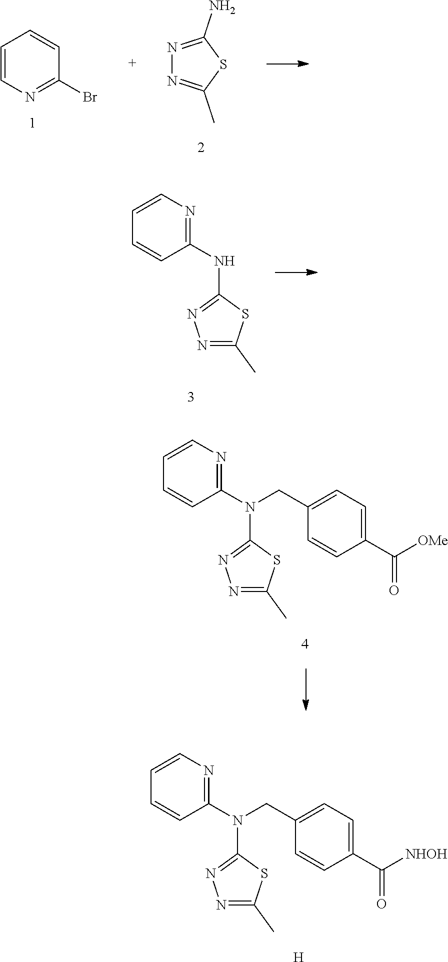

2-Bromopyridine (1) (1.0 g, 6.3 mmol), 5-methyl-1,3,4-thiadiazol-2-amine (2) (0.947 g, 8.2 mmol), Xantphos (0.366 g, 0.63 mmol) and Cs₂CO₃ (3.09 g, 9.4 mmol) were combined in dry 1,4-dioxane (15 mL). The reaction mixture was degassed with N₂(g) and placed under vacuum for 10 min. Pd₂(dba)₃ (0.289 g, 0.31 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide 5-methyl-N-(pyridin-2-yl)-1, 3, 4-thiadiazol-2-amine (3) as a yellow solid (0.22 g, 18%).

LCMS (ES): Found 193.2 [M+H]⁺.

NaH (60%) (109.3 mg, 1.3 mmol) was added portion-wise to 5-methyl-N-(pyridin-2-yl)-1,3,4-thiadiazol-2-amine (3) (500 mg, 2.6 mmol) in DMF (8 mL) at 5° C. under Ar(g). The reaction mixture was stirred for 20 min, then methyl 4-(bromomethyl)benzoate (775 mg, 3.3 mmol) was added and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (1:3) to furnish methyl 4-(((5-methyl-1,3,4-thiadiazol-2-yl)(pyridin-2-yl)amino)methyl)benzoate (4) as a light yellow solid (134 mg, 39%).

LCMS (ES): Found 341.4 [M+H]⁺.

A fresh solution of NH₂OH in MeOH was prepared: [KOH (1.0 g, 19.7 mmol) in MeOH (20 mL) was added to NH₂OH.HCl (1.36 g, 19.7 mmol) in MeOH (20 mL) at 0° C.]. The reaction mixture was stirred for 20 min at 0° C., then filtered to remove salts; it was then added to methyl 4-(((5-methyl-1,3,4-thiadiazol-2-yl)(pyridin-2-yl)amino)methyl)benzoate (4) (134 mg, 0.39 mmol) followed by KOH (221 mg, 3.9 mmol) solubilised in MeOH (10 mL). The reaction mixture was stirred at rt for 21 h, then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-(((5-methyl-1,3,4-thiadiazol-2-yl)(pyridin-2-yl)amino)methyl)benzamide, Example H, as a light brown liquid (15 mg, 11%).

¹H NMR (400 MHz, Methanol-d₄), δ_{H} ppm: 8.42 (dd, J=4.9, 1.1 Hz, 1H), 7.73 (ddd, J=8.6, 7.2, 1.8 Hz, 1H), 7.69 (d, J=8.3 Hz, 2H), 7.33 (d, J=8.2 Hz, 2H), 7.02-7.09 (m, 2H), 5.72 (s, 2H), 2.65 (s, 3H).

LCMS (ES): Found 342.1 [M+H]⁺.

### Example I

### 4-((Benzo[d]oxazol-2-yl(pyridin-2-yl)amino)methyl)-N-hydroxybenzamide

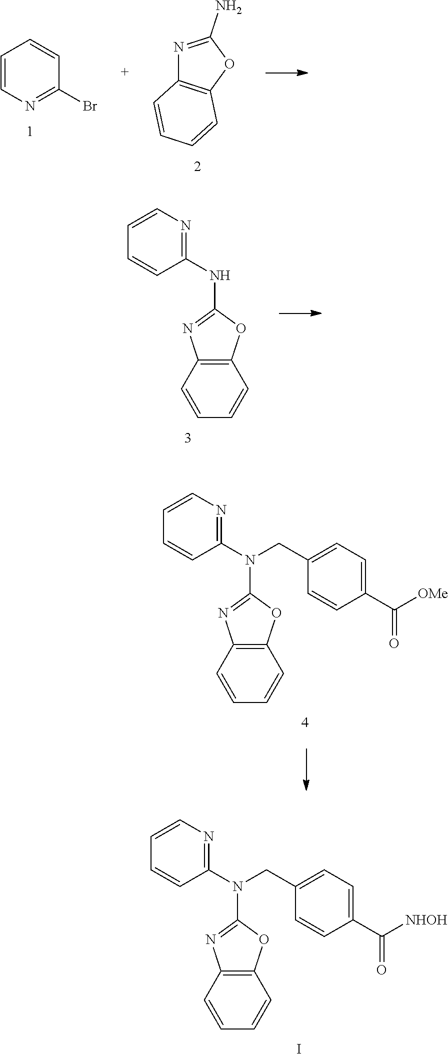

2-Bromopyridine (1) (1.0 g, 6.3 mmol), benzo[d]oxazol-2-amine (2) (0.871 g, 6.4 mmol), Xantphos (0.37 g, 0.63 mmol) and Cs₂CO₃ (3.09 g, 9.4 mmol) were combined in dry 1,4-dioxane (15 mL). The reaction mixture was degassed with N₂(g) and placed under vacuum for 10 min. Pd₂(dba)₃ (0.289 g, 0.31 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(pyridin-2-yl)benzo[d]oxazol-2-amine (3) as a yellow solid (0.8 g, 60%).

LCMS (ES): Found 212.1 [M+H]⁺.

NaH (60%) (53 mg, 1.3 mmol) was added portion-wise to N-(pyridin-2-yl)benzo[d]oxazol-2-amine (3) (265 mg, 1.28 mmol) in DMF (8 mL) at 5° C. under Ar(g). The reaction mixture was stirred for 20 min. then methyl 4-(bromomethyl)benzoate (380 mg, 1.66 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((benzo[d]oxazol-2-yl(pyridin-2-yl)amino)methyl)benzoate (4) as a light yellow solid (220 mg, 48%).

LCMS (ES): Found 360.1 [M+H]⁺.

A fresh solution of NH₂OH in MeOH was prepared: [KOH (1.75 g, 31.0 mmol) in MeOH (15 mL) was added to NH₂OH.HCl (2.16 g, 31.0 mmol) in MeOH (15 mL) at 0° C. The reaction mixture was stirred for 20 min at 0° C., then filtered to remove salts; it was then added to methyl 4-((benzo[d]oxazol-2-yl(pyridin-2-yl)amino)methyl)benzoate (4) (220 mg, 0.62 mmol) followed by KOH (348 mg, 6.2 mmol) solubilised in MeOH (5 mL). The reaction mixture was stirred at rt for 21 h, then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-((benzo[d]oxazol-2-yl(pyridin-2-yl)amino)methyl)-N-hydroxybenzamide, Example I, as a light orange solid (50 mg, 23%).

¹H NMR (400 MHz, DMSO-d₆), δ_{H} ppm: 11.12 (br. s., 1H), 9.00 (br. s., 1H), 8.40 (dd, J=4.7, 1.8 Hz, 1H), 8.17 (d, J=8.4 Hz, 1H), 7.88-7.94 (m, 1H), 7.65 (d, J=8.2 Hz, 2H), 7.47-7.55 (m, 2H), 7.41 (d, J=8.2 Hz, 2H), 7.26 (t, J=7.8 Hz, 1H), 7.14-7.22 (m, 2H), 5.59 (s, 2H).

LCMS (ES): Found 361.1 [M+H]⁺.

### Example J

### N-Hydroxy-4-(((1-methyl-1H-benzo[d]imidazol-2-yl)pyridin-2-yl)amino)methyl)benzamide

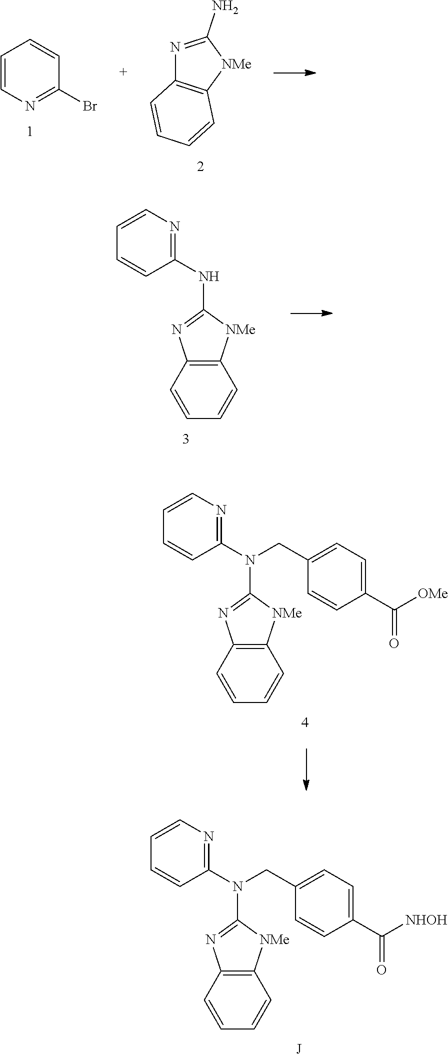

2-Bromopyridine (1) (1.0 g, 6.3 mmol), 1-methyl-1H-pyrazol-3-amine (2) (1.21 g, 6.9 mmol), Xantphos (0.37 g, 0.63 mmol) and Cs₂CO₃ (4.1 g, 12.6 mmol) were combined in dry 1,4-dioxane (15 mL). The reaction mixture was degassed with N₂(g) and placed under vacuum for 10 min. Pd₂(dba)₃ (0.29 g, 0.31 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide 1-methyl-N-(pyridin-2-yl)-1H-benzo[d]imidazol-2-amine (3) as a yellow solid (0.35 g, 25%).

LCMS (ES): Found 225.1 [M+H]⁺.

NaH (60%) (32.8 mg, 0.82 mmol) was added portion-wise to 1-methyl-N-(pyridin-2-yl)-1H-benzo[d]imidazol-2-amine (3) (175 mg, 0.78 mmol) in DMF (5 mL) at 5° C. under Ar(g). The reaction mixture was stirred for 20 min, then methyl 4-(bromomethyl) benzoate (232 mg, 1.01 mmol) was added, and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The residue was purified by flash chromatography with EtOAc/Hexane (3:7) to furnish methyl 4-(((1-methyl-1H-benzo[d]imidazol-2-yl)(pyridin-2-yl)amino)methyl)benzoate (4) as a light yellow solid (42 mg, 16%).

LCMS (ES): Found 373.2 [M+H]⁺.

A fresh solution of NH₂OH in MeOH was prepared: [KOH (1.07 g, 19.0 mmol) in MeOH (10 mL) was added to NH₂OH.HCl (530 mg, 19.0 mmol) in MeOH (10 mL) at 0° C.]. The reaction mixture was stirred for 20 min at 0° C., then filtered to remove salts; it was then added to methyl 4-(((1-methyl-1H-benzo[d]imidazol-2-yl)(pyridin-2-yl)amino)methyl)benzoate (4) (142 mg, 0.38 mmol) followed by KOH (214 mg, 3.8 mmol) solubilised in MeOH (5 mL). The reaction mixture was stirred at rt for 21 h, and then concentrated in vacuo, poured onto brine/H₂O (30 mL/70 mL), and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (10:90) to provide N-hydroxy-4-(((1-methyl-1H-benzo[d]imidazol-2-yl)(pyridin-2-yl)amino)methyl)benzamide, Example J, as an off white solid (9 mg, 7%).

¹H NMR (400 MHz, Methanol-d₄), δ_{H} ppm: 8.23 (dd, J=5.0, 1.1 Hz, 1H), 7.65 (d, J=8.3 Hz, 2H), 7.58-7.63 (m, 2H), 7.52 (d, J=8.2 Hz, 2H), 7.41 (dd, J=6.8, 1.9 Hz, 1H), 7.24-7.32 (m, 2H), 6.92 (dd, J=6.8, 5.1 Hz, 1H), 6.56 (d, J=8.4 Hz, 1H), 5.37 (s, 2H), 3.37-3.42 (m, 3H).

LCMS (ES): Found 374.3 [M+H]⁺.

### Example K

### N-Hydroxy-4-((pyridin-2-yl(1,2,4-thiadiazol-5-yl)amino)methyl)benzamide

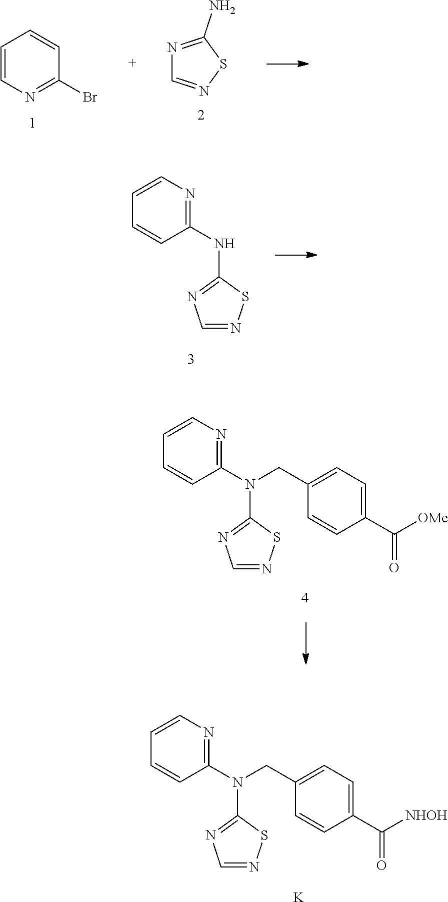

2-Bromopyridine (1) (1.0 g, 6.3 mmol), 1, 2, 4-thiadiazol-5-amine (2) (0.830 g, 8.22 mmol), Xantphos (0.366 g, 0.63 mmol), and Cs₂CO₃ (3.09 g, 9.4 mmol) were combined in dry 1,4-dioxane (15 mL). The reaction mixture was degassed with N₂(g) and placed under vacuum for 10 min. Pd₂(dba)₃ (0.29 g, 0.31 mmol) was then added and the resulting reaction mixture was heated at 90° C. for 30 h. It was then poured onto demineralised water (200 mL), and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(pyridin-2-yl)-1,2,4-thiadiazol-5-amine (3) as a yellow solid (0.188 g, 16%).

LCMS (ES): Found 179.0 [M+H]⁺

NaH (60%) (49 mg, 1.23 mmol) was added portion-wise to N-(pyridin-2-yl)-1,2,4-thiadiazol-5-amine (3) (210 mg, 1.19 mmol) in DMF (8 mL) at 5° C. under Ar(g). The reaction mixture was stirred for 20 min, then methyl 4-(bromomethyl)benzoate (351 mg, 1.5 mmol) was added and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((pyridin-2-yl(1,2,4-thiadiazol-5-yl)amino)methyl)benzoate (4) as a light yellow solid (110 mg, 28%).

LCMS (ES): Found 327.4 [M+H]⁺.

A fresh solution of NH₂OH in MeOH was prepared: [KOH (949 mg, 16.9 mmol) in MeOH (10 mL) was added to NH₂OH.HCl (1.17 g, 16.9 mmol) in MeOH (10 mL) at 0° C.]. The reaction mixture was stirred for 20 min at 0° C., then filtered to remove salts; it was then added to methyl 4-((pyridin-2-yl(1,2,4-thiadiazol-5-yl)amino)methyl)benzoate (4) (110 mg, 0.33 mmol) followed by KOH (185 mg, 3.3 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-((pyridin-2-yl(1,2,4-thiadiazol-5-yl)amino)methyl)benzamide, Example K, as a light orange solid (11 mg, 10%).

¹H NMR (400 MHz, Methanol-d₄), δ_{H} ppm: 8.54 (d, J=4.3 Hz, 1H), 8.22-8.31 (m, 1H), 7.81 (br. s., 1H), 7.65-7.76 (m, 2H), 7.08-7.38 (m, 4H), 5.82 (s, 2H).

LCMS (ES): Found 328.0 [M+H]⁺.

### Example L

### 4-(5-Fluoropyridin-2-yl)pyrazin-2-yl)amino)methyl)-N-hydroxybenzamide

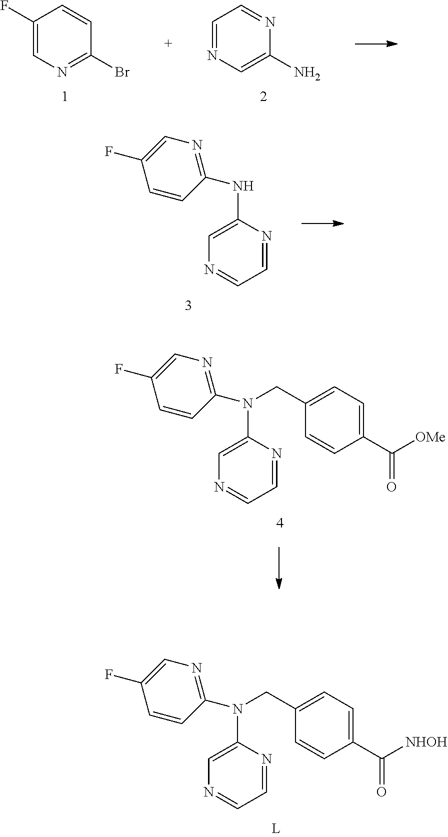

2-Bromo-5-fluoropyridine (1) (1.0 g, 5.71 mmol), pyrazin-2-amine (2) (543 mg, 5.71 mmol), Xantphos (0.330 g, 0.57 mmol), Cs₂CO₃ (2.79 g, 8.56 mmol) were combined in dry 1,4-dioxane (15 mL). The reaction mixture was degassed with N₂(g), and placed under vacuum for 10 min. Pd₂(dba)₃ (0.26 g, 0.28 mmol) was added and the reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(5-fluoropyridin-2-yl)pyrazin-2-amine (3) as a yellow solid (0.56 g, 51%).

LCMS (ES): Found 191.1 [M+H]⁺.

NaH (60%) (39 mg, 0.99 mmol) was added portion-wise to N-(5-fluoropyridin-2-yl)pyrazin-2-amine (3) (180 mg, 0.94 mmol) in DMF (5 mL) at 5° C. under Ar(g). The reaction mixture was stirred for 20 min, then methyl 4-(bromomethyl) benzoate (281 mg, 1.23 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((5-fluoropyridin-2-yl)(pyrazin-2-yl)amino)methyl)benzoate (4) as a light yellow solid (190 mg, 59%).

LCMS (ES): Found 339.1 [M+H]⁺.

A fresh solution of NH₂OH in MeOH was prepared: [KOH (1.57 g, 28.1 mmol) in MeOH (15 mL) was added to NH₂OH.HCl (1.95 g, 28.1 mmol) in MeOH (15 mL) at 0° C.]. The reaction mixture was stirred for 20 min at 0° C., then filtered to remove salts; it was then added to methyl 4-(((5-fluoropyridin-2-yl)(pyrazin-2-yl)amino)methyl)benzoate (4) (190 mg, 0.56 mmol) followed by KOH (315 mg, 5.6 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(pyrazin-2-yl)amino)methyl)-N-hydroxybenzamide, Example L, as a creamish solid (40 mg, 21%).

¹H NMR (400 MHz, DMSO-d₆), δ_{H} ppm: 11.08 (br. s, 1H), 8.84-9.09 (m, 1H), 8.54 (d, J=1.4 Hz, 1H), 8.34 (d, J=3.1 Hz, 1H), 8.24 (dd, J=2.7, 1.5 Hz, 1H), 8.09 (d, J=2.7 Hz, 1H), 7.72 (ddd, J=9.0, 8.2, 3.1 Hz, 1H), 7.64 (d, J=8.3 Hz, 2H), 7.46 (dd, J=9.1, 3.7 Hz, 1H), 7.37 (d, J=8.3 Hz, 2H), 5.42 (s, 2H).

LCMS (ES): Found 340.1 [M+H]⁺.

### Example M

### 4-(((5-Fluoropyridin-2-yl)(3-methyl-1,2,4-oxadiazol-5-yl)amino)methyl)-N-hydroxybenzamide

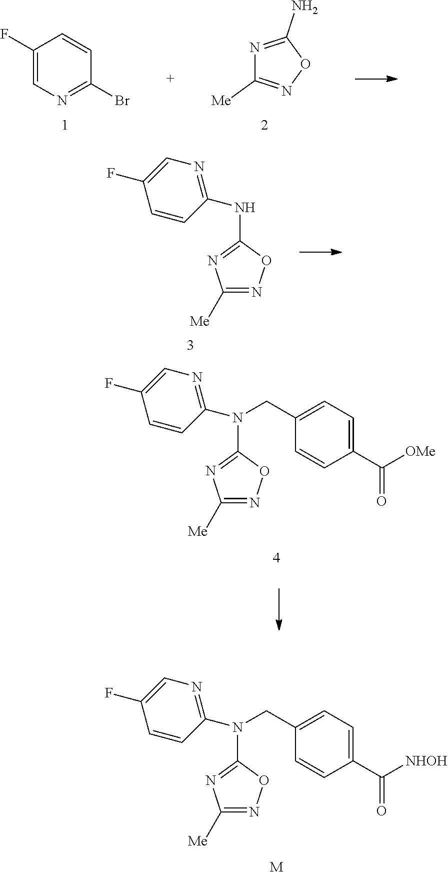

2-Bromo-5-fluoropyridine (1) (1.0 g, 5.71 mmol), 3-methyl-1,2,4-oxadiazol-5-amine (2) (566 mg, 5.71 mmol), Xantphos (0.330 g, 0.57 mmol) and Cs₂CO₃ (2.79 g, 8.56 mmol) were combined in dry 1,4-dioxane (15 mL). The reaction mixture was degassed with N₂(g) and placed under vacuum for 10 min. Pd₂(dba)₃ (0.261 g, 0.28 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(5-fluoropyridin-2-yl)-3-methyl-1,2,4-oxadiazol-5-amine (3) as a yellow solid (0.70 g, 63%).

LCMS (ES): Found 195.0 [M+H]⁺.

NaH (60%) (56 mg, 1.4 mmol) was added portion-wise to N-(5-fluoropyridin-2-yl)-3-methyl-1,2,4-oxadiazol-5-amine (3) (260 mg, 1.34 mmol) in DMF (10 mL) at 5° C. under Ar(g). The reaction mixture was stirred for 20 min, then methyl 4-(bromomethyl) benzoate (398 mg, 1.7 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl-4-(((5-fluoropyridin-2-yl)(3-methyl-1,2,4-oxadiazol-5-yl)amino)methyl)benzoate (4) as a light yellow solid (170 mg, 37%).

LCMS (ES): Found 343.1 [M+H]⁺.

A fresh solution of NH₂OH in MeOH was prepared: [KOH (1.39 g, 24.8 mmol) in MeOH (15 mL) was added to NH₂OH.HCl (1.72 g, 24.8 mmol) in MeOH (15 mL) at 0° C.]. The reaction mixture was stirred for 20 min at 0° C., then filtered to remove salts; it was then added to methyl 4-(((5-fluoropyridin-2-yl)(3-methyl-1,2,4-oxadiazol-5-yl)amino)methyl)benzoate (4) (170 mg, 0.49 mmol) followed by KOH (278 mg, 4.9 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(3-methyl-1,2,4-oxadiazol-5-yl)amino)methyl)-N-hydroxybenzamide, Example M, as a light orange solid (20 mg, 12%).

¹H NMR (400 MHz, DMSO-d₆), δ_{H} ppm: 11.11 (br. s., 1H), 9.01 (br. s., 1H), 8.43 (d, J=3.0 Hz, 1H), 8.11 (dd, J=9.2, 3.8 Hz, 1H), 7.89 (td, J=8.6, 3.1 Hz, 1H), 7.67 (d, J=8.3 Hz, 2H), 7.34 (d, J=8.2 Hz, 2H), 5.43 (s, 2H), 2.22 (s, 3H).

LCMS (ES): Found 344.1 [M+H]⁺.

### Example N

### 4-((5-Fluoropyridin-2-yl)(1-methyl-1H-benzo[d]imidazol-2-yl)amino)methyl)-N-hydroxybenzamide

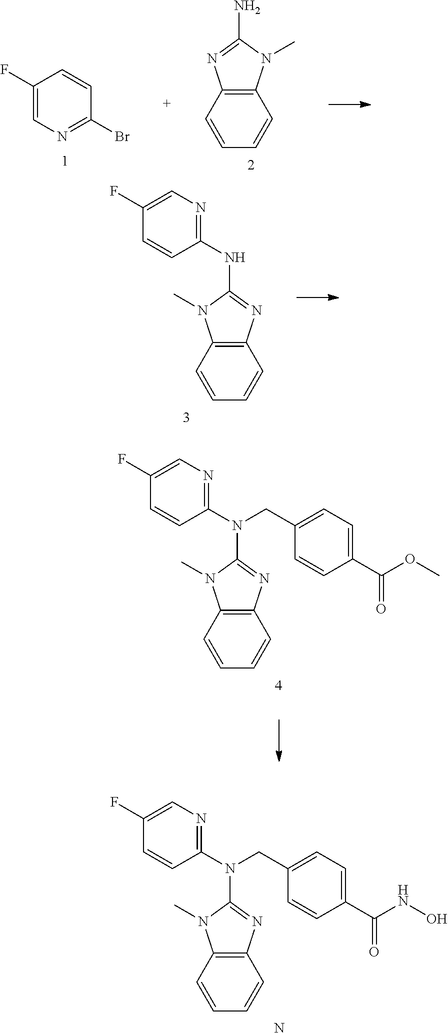

2-Bromo-5-fluoropyridine (1) (1.0 g, 5.71 mmol), 1-methyl-1H-benzo[d]imidazol-2-amine (2) (840 mg, 5.71 mmol), Xantphos (0.33 g, 0.57 mmol) and Cs₂CO₃ (2.79 g, 8.56 mmol) were combined in dry 1,4-dioxane (15 mL). The reaction mixture was degassed with N₂(g) and placed under vacuum for 10 min. Pd₂(dba)₃ (0.26 g, 0.28 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(5-fluoropyridin-2-yl)-1-methyl-1H-benzo[d]imidazol-2-amine (3) as a yellow solid (0.56 g, 41%).

LCMS (ES): Found 243.1 [M+H]⁺.

NaH (60%) (27 mg, 0.66 mmol) was added portion-wise to N-(5-fluoropyridin-2-yl)-1-methyl-1H-benzo[d]imidazol-2-amine (3) (154 mg, 0.63 mmol) in DMF (5 mL) at 5° C. under Ar(g). The reaction mixture was stirred for 20 min, then methyl 4-(bromomethyl) benzoate (189 mg, 0.82 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((5-fluoropyridin-2-yl)(1-methyl-1H-benzo[d]imidazol-2-yl)amino)methyl)benzoate (4) as a light yellow solid (165 mg, 66%).

LCMS (ES): Found 391.2 [M+H]⁺.

A fresh solution of NH₂OH in MeOH was prepared: [KOH (1.20 g, 21.4 mmol) in MeOH (15 mL) was added to NH₂OH.HCl (1.48 g, 21.4 mmol) in MeOH (15 mL) at 0° C.]. The reaction mixture was stirred for 20 min at 0° C., then filtered to remove salts; it was then added to methyl 4-(((5-fluoropyridin-2-yl)(1-methyl-1H-benzo[d]imidazol-2-yl)amino)methyl)benzoate (4) (165 mg, 0.40 mmol) followed by KOH (240 mg, 4.0 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(1-methyl-1H-benzo[d]imidazol-2-yl)amino)methyl)-N-hydroxybenzamide, Example N, as a light orange solid (20 mg, 12%).

¹H NMR (400 MHz, DMSO-d₆), δ_{H} ppm: 8.19 (d, J=2.9 Hz, 1H), 7.66 (d, J=8.2 Hz, 1H), 7.55-7.63 (m, 3H), 7.42-7.54 (m, 3H), 7.15-7.27 (m, 2H), 6.74 (dd, J=9.2, 3.4 Hz, 1H), 5.22-5.31 (m, 2H), 3.42 (s, 3H).

LCMS (ES): Found 392.25 [M+H]⁺.

### Example O

### 4-((5-Fluoropyridin-2-yl)(1-methyl-1H-pyrazol-3-yl)amino)methyl)-N-hydroxybenzamide

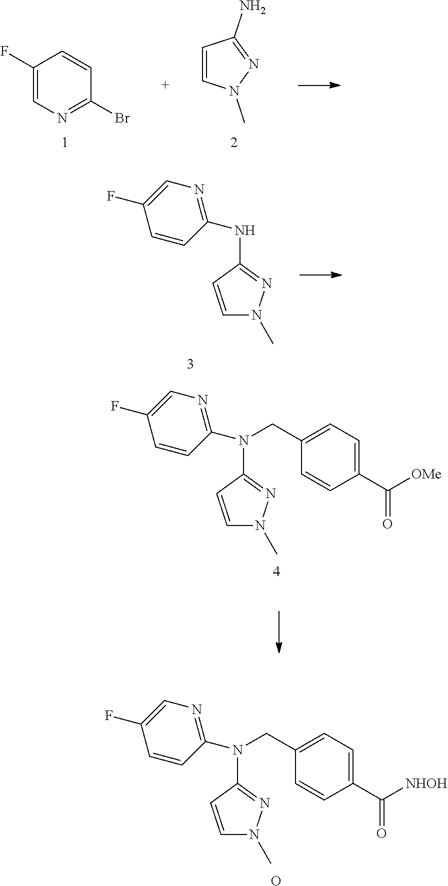

2-Bromo-5-fluoropyridine (1) (1.0 g, 5.71 mmol), 1-methyl-1H-pyrazol-3-amine (2) (554 mg, 5.71 mmol), Xantphos (0.330 g, 0.57 mmol) and Cs₂CO₃ (2.79 g, 8.56 mmol) were combined in dry 1,4-dioxane (15 mL). The reaction mixture was degassed with N₂(g) and placed under vacuum for 10 min. Pd₂(dba)₃ (0.261 g, 0.28 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide 5-fluoro-N-(1-methyl-1H-pyrazol-3-yl)pyridin-2-amine (3) as a yellow solid (0.65 g, 61%).

LCMS (ES): Found 193.0 [M+H]⁺.

NaH (60%) (50 mg, 1.25 mmol) was added portion-wise to 5-fluoro-N-(1-methyl-1H-pyrazol-3-yl)pyridin-2-amine (3) (230 mg, 1.19 mmol) in DMF (10 mL) at 5° C. under Ar(g). The reaction mixture was stirred for 20 min, then methyl 4-(bromomethyl) benzoate (356 mg, 1.55 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((5-fluoropyridin-2-yl)(1-methyl-1H-pyrazol-3-yl)amino)methyl)benzoate (4) as a light yellow solid (312 mg, 76%).

LCMS (ES): Found 341.1 [M+H]⁺.

A fresh solution of NH₂OH in MeOH was prepared: [KOH (2.57 g, 45.8 mmol) in MeOH (15 mL) was added to NH₂OH.HCl (3.18 g, 45.8 mmol) in MeOH (15 mL) at 0° C.]. The reaction mixture was stirred for 20 min at 0° C., then filtered to remove salts; it was then added to methyl methyl 4-(((5-fluoropyridin-2-yl)(1-methyl-1H-pyrazol-3-yl)amino)methyl)benzoate (4) (312 mg, 0.91 mmol) followed by KOH (512 mg, 9.1 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(1-methyl-1H-pyrazol-3-yl)amino)methyl)-N-hydroxybenzamide, Example O, as a cream solid (65 mg, 20%).

¹H NMR (400 MHz, DMSO-d₆), δ_{H} ppm: 11.11 (br. s, 1H), 8.96 (br. s, 1H), 8.10 (d, J=3.1 Hz, 1H), 7.59-7.66 (m, 3H), 7.51 (ddd, J=9.3, 8.2, 3.1 Hz, 1H), 7.31 (d, J=8.1 Hz, 2H), 7.19 (dd, J=9.4, 3.7 Hz, 1H), 6.13 (d, J=2.3 Hz, 1H), 5.21 (s, 2H), 3.76 (s, 3H).

LCMS (ES): Found 342.1 [M+H]⁺.

### Example P

### 4-((Benzo[d]oxazol-2-yl(5-fluoropyridin-2-yl)amino)methyl)-N-hydroxybenzamide

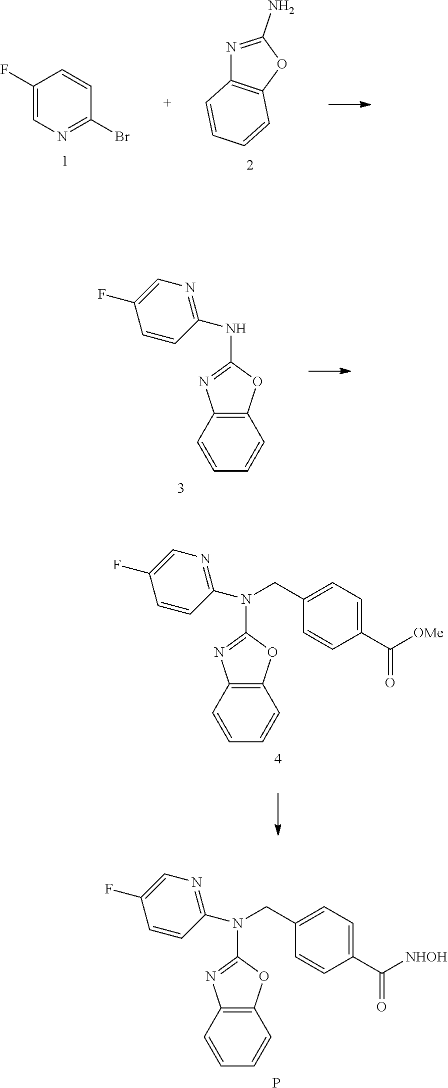

2-Bromo-5-fluoropyridine (1) (1.0 g, 5.71 mmol), benzo[d]oxazol-2-amine (2) (766 mg, 5.71 mmol), Xantphos (0.33 g, 0.57 mmol) and Cs₂CO₃ (2.79 g, 8.56 mmol) were combined in dry 1,4-dioxane (15 mL). The reaction mixture was degassed with N₂(g) and placed under vacuum for 10 min. Pd₂(dba)₃ (0.261 g, 0.28 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(5-fluoropyridin-2-yl)benzo[d]oxazol-2-amine (3) as a yellow solid (0.6 g, 46%).

LCMS (ES): Found 230.1 [M+H]⁺.

NaH (60%) (36 mg, 0.91 mmol) was added portion-wise to N-(5-fluoropyridin-2-yl)benzo[d]oxazol-2-amine (3) (200 mg, 0.87 mmol) in DMF (8 mL) at 5° C. under Ar(g). The reaction mixture was stirred for 20 min, then methyl 4-(bromomethyl) benzoate (259 mg, 1.13 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((benzo[d]oxazol-2-yl(5-fluoropyridin-2-yl)amino)methyl)benzoate (4) as a light yellow solid (144 mg, 43%).

LCMS (ES): Found 378.1 [M+H]⁺.

A fresh solution of NH₂OH in MeOH was prepared: [KOH (1.07 g, 19.0 mmol) in MeOH (15 mL) was added to NH₂OH.HCl (1.33 g, 19.0 mmol) in MeOH (15 mL) at 0° C.]. The reaction mixture was stirred for 20 min at 0° C., then filtered to remove salts; it was then added to methyl 4-((benzo[d]oxazol-2-yl(5-fluoropyridin-2-yl)amino)methyl)benzoate (4) (144 mg, 0.38 mmol) followed by KOH (214 mg, 3.8 mmol) solubilised in MeOH (5 mL). The reaction mixture was stirred at rt for 21 h then concentrated in vacuo, poured onto brine/H₂O (30 mL/70 mL), and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-((benzo[d]oxazol-2-yl(5-fluoropyridin-2-yl)amino)methyl)-N-hydroxybenzamide, Example P, as an orange solid (30 mg, 20%).

¹H NMR (400 MHz, DMSO-d₆), δ_{H} ppm: 11.13 (br. s, 1H), 9.01 (br. s., 1H), 8.41 (d, J=3.1 Hz, 1H), 8.25 (dd, J=9.2, 3.8 Hz, 1H), 7.89 (ddd, J=9.2, 8.1, 3.1 Hz, 1H), 7.66 (d, J=8.3 Hz, 2H), 7.47-7.54 (m, 2H), 7.41 (d, J=8.2 Hz, 2H), 7.26 (td, J=7.7, 1.1 Hz, 1H), 7.13-7.20 (m, 1H), 5.54 (s, 2H).

LCMS (ES): Found 379.1 [M+H]⁺.

### Example Q

4-(((4-(4-Fluorophenyl)pyridin-2-yl)(1-methyl-1H-pyrazol-3-yl)amino)methyl)-N-hydroxybenzamide

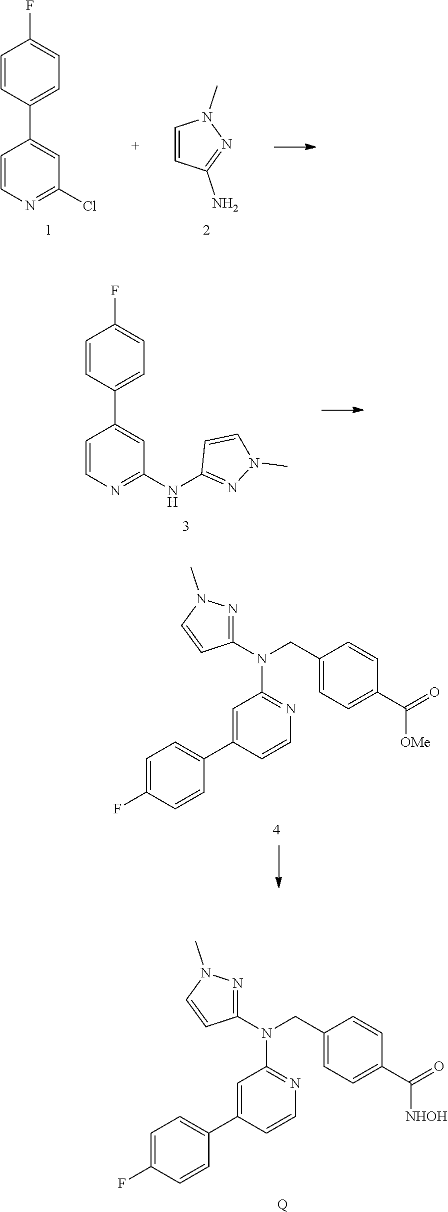

2-Chloro-4-(4-fluorophenyl)pyridine (1) (1.0 g, 4.8 mmol), 1-methyl-1H-pyrazol-3-amine (2) (470 mg, 4.8 mmol), Xantphos (0.28 g, 0.48 mmol) and Cs₂CO₃ (2.35 g, 7.24 mmol) were combined in dry 1,4-dioxane (15 mL). The reaction mixture was degassed with N₂(g) and placed under vacuum for 10 min. Pd₂(dba)₃ (0.22 g, 0.24 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide 4-(4-fluorophenyl)-N-(1-methyl-1H-pyrazol-3-yl)pyridin-2-amine (3) as a yellow solid (1.0 g, 71%).

LCMS (ES): Found 269.1 [M+H]⁺.

NaH (60%) (37 mg, 0.93 mmol) was added portion-wise to 4-(4-fluorophenyl)-N-(1-methyl-1H-pyrazol-3-yl)pyridin-2-amine (3) (250 mg, 0.93 mmol) in DMF (10 mL) at 5° C. under Ar(g). The reaction mixture was stirred for 20 min, then methyl 4-(bromomethyl) benzoate (277 mg, 1.2 mmol) was added and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((4-(4-fluorophenyl)pyridin-2-yl)(1-methyl-1H-pyrazol-3-yl)amino)methyl)benzoate (4) as a light yellow solid (267 mg, 68%).

LCMS (ES): Found 417.4 [M+H]⁺.

A fresh solution of NH₂OH in MeOH was prepared: [KOH (1.79 g, 32.0 mmol) in MeOH (15 mL) was added to NH₂OH.HCl (2.23 g, 32.0 mmol) in MeOH (15 mL) at 0° C.]. The reaction mixture was stirred for 20 min at 0° C., then filtered to remove salts; it was then added to methyl 4-(((4-(4-fluorophenyl)pyridin-2-yl)(1-methyl-1H-pyrazol-3-yl)amino)methyl)benzoate (4) (267 mg, 0.64 mmol) followed by KOH (359 mg, 6.41 mmol) solubilised in MeOH (10 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to 4-(((4-(4-fluorophenyl)pyridin-2-yl)(1-methyl-1H-pyrazol-3-yl)amino)methyl)-N-hydroxybenzamide, Example Q, as an off white solid (30 mg, 11%).

¹H NMR (400 MHz, DMSO-d₆), δ_{H} ppm: 11.11 (br. s, 1H), 9.00 (br. s, 1H), 8.19 (d, J=5.3 Hz, 1H), 7.59-7.71 (m, 5H), 7.24-7.39 (m, 5H), 6.98-7.05 (m, 1H), 6.26 (d, J=2.2 Hz, 1H), 5.30 (s, 2H), 3.74-3.79 (m, 3H).

LCMS (ES): Found 418.2 [M+H]⁺.

### Example R

### 4-((5-Fluoropyridin-2-yl)(3-methyl-1,2,4-thiadiazol-5-yl)amino)methyl)-N-hydroxybenzamide

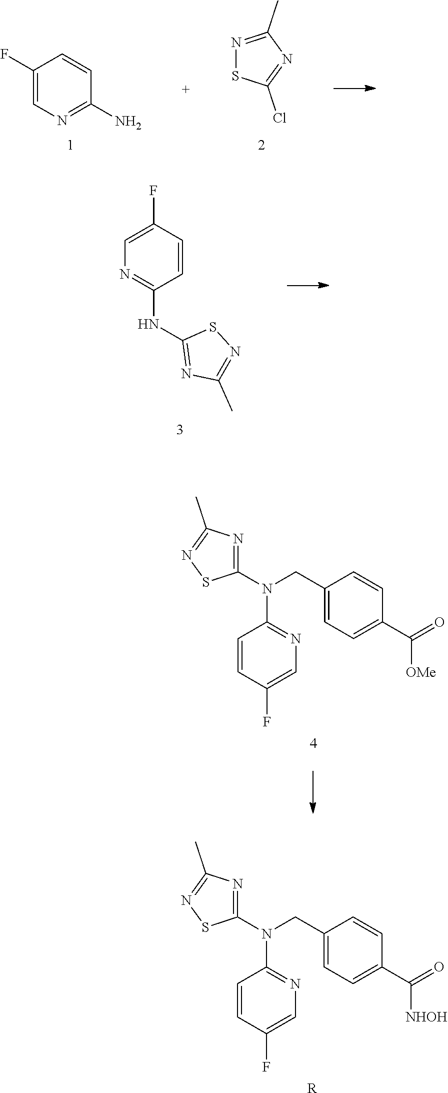

5-Fluoropyridin-2-amine (1) (1.0 g, 8.9 mmol), 5-chloro-3-methyl-1,2,4-thiadiazole (2) (1.19 g, 8.9 mmol), Xantphos (0.52 g, 0.89 mmol) and Cs₂CO₃ (4.35 g, 13.3 mmol) were combined in dry 1,4-dioxane (15 mL). The reaction mixture was degassed with N₂(g) and placed under vacuum for 10 min. Pd₂(dba)₃ (0.41 g, 0.44 mmol) was then added and the resulting reaction mixture was heated at 90° C. for 30 h. The reaction mixture was then poured onto demineralised water (200 mL), and extracted with EtOAc (3×100 mL). The organic phases were combined, dried over Na₂SO₄, filtered and subsequently concentrated in vacuo. The resulting residue was purified by flash chromatography with EtOAc/Hexane (3:7) to provide N-(5-fluoropyridin-2-yl)-3-methyl-1,2,4-thiadiazol-5-amine (3) as a yellow solid (1.2 g, 67%).

LCMS (ES): Found 211.1 [M+H]⁺.

NaH (60%) (59 mg, 1.49 mmol) was added portion-wise to N-(5-fluoropyridin-2-yl)-3-methyl-1,2,4-thiadiazol-5-amine (3) (300 mg, 1.42 mmol) in DMF (7 mL) at 5° C. under Ar(g). The reaction mixture was stirred for 20 min, then methyl 4-(bromomethyl) benzoate (425 mg, 1.85 mmol) was added and stirring was continued at 70° C. under Ar(g) for 1 h in the dark. The reaction mixture was then poured onto water (100 mL), and extracted with EtOAc (3×50 mL). The organic phases were combined, dried over Na₂SO₄, filtered and subsequently concentrated in vacuo. The residue was purified by flash chromatography with EtOAc/Hexane (3:7) to furnish methyl-4-(((5-fluoropyridin-2-yl)(3-methyl-1,2,4-thiadiazol-5-yl)amino)methyl)benzoate (4) as a yellow solid (480 mg, 90%).

LCMS (ES): Found 359.3 [M+H]⁺.

A fresh solution of NH₂OH in MeOH was prepared: [KOH (4.63 g, 67.0 mmol) in MeOH (20 mL) was added to NH₂OH.HCl (3.76 g, 67.0 mmol) in MeOH (20 mL) at 0° C.]. The reaction mixture was stirred for 20 min at 0° C., then filtered to remove salts; it was then added to methyl 4-(((5-fluoropyridin-2-yl)(3-methyl-1,2,4-thiadiazol-5-yl)amino)methyl)benzoate (4) (480 mg, 1.3 mmol) followed by KOH (750 mg, 1.3 mmol) solubilised in MeOH (10 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(3-methyl-1,2,4-thiadiazol-5-yl)amino)methyl)-N-hydroxybenzamide, Example R, as an orange solid (90 mg, 19%).

¹H NMR (400 MHz, DMSO-d₆), δ_{H} ppm: 11.16 (br. s., 1H), 9.03 (br. s., 1H), 8.60 (d, J=2.9 Hz, 1H), 7.86 (td, J=8.7, 2.8 Hz, 1H), 7.64-7.76 (m, 2H), 7.19-7.34 (m, 3H), 5.77 (s, 2H), 2.39 (s, 3H).

LCMS (ES): Found 359.8 [M+H]⁺.

### Example S

### 4-(((4-(4-Fluorophenyl)pyridin-2-yl)(3-methyl-1,2,4-thiadiazol-5-yl)amino)methyl)-N-hydroxybenzamide

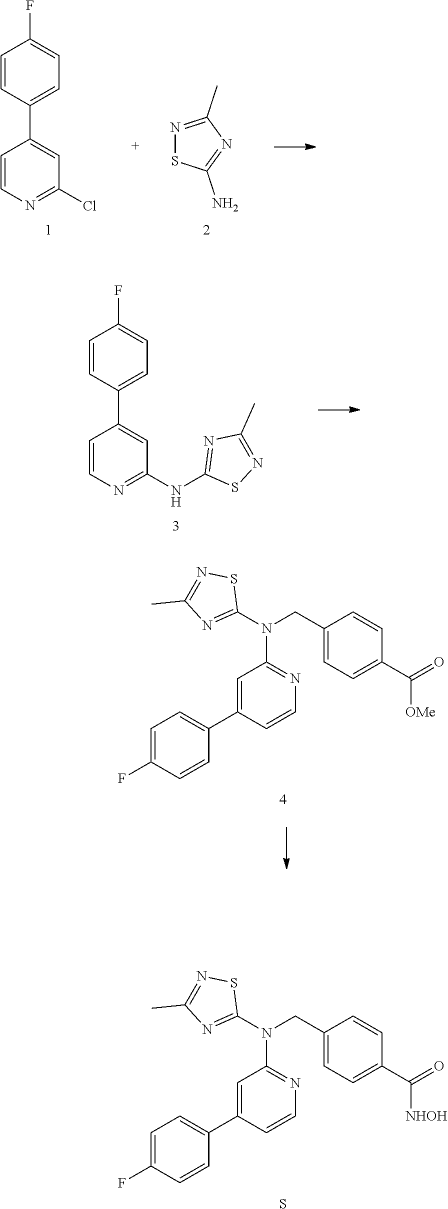

2-Chloro-4-(4-fluorophenyl)pyridine (1) (1.0 g, 4.8 mmol), 3-methyl-1, 2, 4-thiadiazol-5-amine (2) (0.56 g, 4.8 mmol), Xantphos (0.279 g, 0.48 mmol) and Cs₂CO₃ (2.35 g, 7.24 mmol) were combined in dry 1,4-dioxane (15 mL). The reaction mixture was degassed with N₂(g) and placed under vacuum for 10 min. Pd₂(dba)₃ (0.22 g, 0.24 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(4-(4-fluorophenyl)pyridin-2-yl)-3-methyl-1,2,4-thiadiazol-5-amine (3) as a yellow solid (1.1 g, 80%).

LCMS (ES): Found 287.1 [M+H]⁺.

NaH (60%) (42 mg, 1.05 mmol) was added portion-wise to N-(4-(4-fluorophenyl)pyridin-2-yl)-3-methyl-1,2,4-thiadiazol-5-amine (3) (300 mg, 1.05 mmol) in DMF (10 mL) at 5° C. under Ar(g). The reaction mixture was stirred for 20 min, then methyl 4-(bromomethyl)benzoate (312 mg, 1.36 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((4-(4-fluorophenyl)pyridin-2-yl)(3-methyl-1,2,4-thiadiazol-5-yl)amino)methyl)benzoate (4) as a yellow solid (325 mg, 74%).

LCMS (ES): Found 421.1 [M+H]⁺.

A fresh solution of NH₂OH in MeOH was prepared: [KOH (1.96 g, 35 mmol) in MeOH (10 mL) was added to NH₂OH.HCl (2.43 g, 35 mmol) in MeOH (10 mL) at 0° C.]. The reaction mixture was stirred for 20 min at 0° C., then filtered to remove salts; it was then added to methyl 4-(((4-(4-fluorophenyl)pyridin-2-yl)(3-methyl-1,2,4-thiadiazol-5-yl)amino)methyl)benzoate (4) (319 mg, 0.69 mmol) followed by KOH (392 mg, 7.0 mmol) solubilised in MeOH (10 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to 4-(((4-(4-fluorophenyl)pyridin-2-yl)(3-methyl-1,2,4-thiadiazol-5-yl)amino)methyl)-N-hydroxybenzamide, Example S, as an off white solid (58 mg, 19%).

¹H NMR (400 MHz, DMSO-d₆), δ_{H} ppm: 11.13 (br. s., 1H), 9.02 (br. s., 1H), 8.59 (d, J=5.3 Hz, 1H), 7.82 (dd, J=8.7, 5.3 Hz, 2H), 7.67 (d, J=8.2 Hz, 2H), 7.43-7.51 (m, 2H), 7.27-7.40 (m, 4H), 5.92 (s, 2H), 2.40 (s, 3H).

LCMS (ES): Found 436.4 [M+H]⁺.

### Example T

### 4-((5-Fluoropyridin-2-yl)(3-(trifluoromethyl)-1,2,4-thiadiazol-5-yl)amino)methyl)-N-hydroxybenzamide

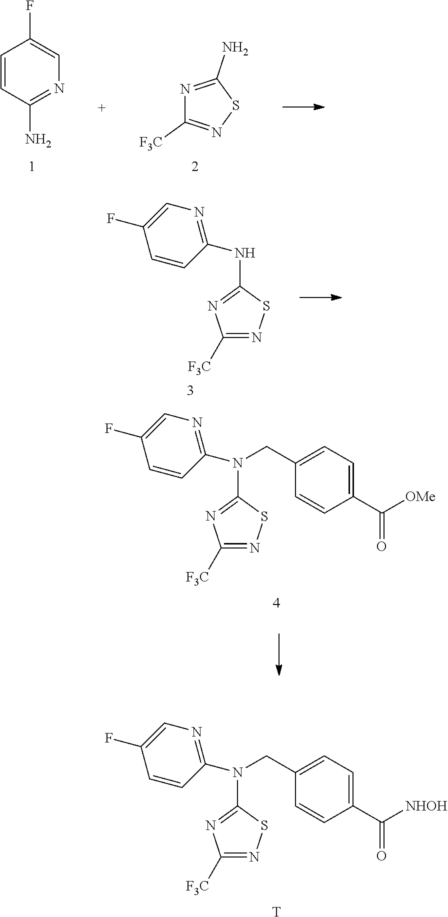

5-Fluoropyridin-2-amine (1) (1.0 g, 8.9 mmol), 5-chloro-3-(trifluoromethyl)-1,2,4-thiadiazole (2) (1.68 g, 8.9 mmol), Xantphos (0.52 g, 0.89 mmol), and Cs₂CO₃ (4.35 g, 13.3 mmol) were combined in dry 1,4-dioxane (15 mL). The reaction mixture was degassed with N₂(g) and placed under vacuum for 10 min. Pd₂(dba)₃ (0.41 g, 0.44 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (3:7) to provide N-(5-fluoropyridin-2-yl)-3-(trifluoromethyl)-1, 2, 4-thiadiazol-5-amine (3) as a yellow solid (900 mg, 38%).

LCMS (ES): Found 265.1 [M+H]⁺.

NaH (60%) (61 mg, 1.51 mmol) was added portion-wise to N-(5-fluoropyridin-2-yl)-3-(trifluoromethyl)-1,2,4-thiadiazol-5-amine (3) (400 mg, 1.51 mmol) in DMF (10 mL) at 5° C. under Ar(g). The reaction mixture was stirred for 20 min, then methyl 4-(bromomethyl) benzoate (451 mg, 1.85 mmol) was added and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((5-fluoropyridin-2-yl)(3-(trifluoromethyl)-1,2,4-thiadiazol-5-yl)amino)methyl)benzoate (3) as a yellow solid (535 mg, 82%).

LCMS (ES): Found 413.3 [M+H]⁺.

A fresh solution of NH₂OH in MeOH was prepared: [KOH (3.63 g, 64.0 mmol) in MeOH (20 mL) was added to NH₂OH.HCl (4.47 g, 64.0 mmol) in MeOH (20 mL) at 0° C.]. The reaction mixture was stirred for 20 min at 0° C., then filtered to remove salts; it was then added to methyl 4-(((5-fluoropyridin-2-yl)(3-(trifluoromethyl)-1,2,4-thiadiazol-5-yl)amino)methyl)benzoate (3) (535 mg, 1.2 mmol) followed by KOH (720 mg, 13.0 mmol) solubilised in MeOH (10 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-(((5-fluoropyridin-2-yl)(3-(trifluoromethyl)-1,2,4-thiadiazol-5-yl)amino)methyl)-N-hydroxybenzamide, Example T, as an orange solid (90 mg, 17%).

¹H NMR (400 MHz, DMSO-d₆), δ_{H} ppm: 11.18 (br. s., 1H), 9.06 (br. s., 1H), 8.73 (d, J=2.7 Hz, 1H), 7.97 (td, J=8.6, 2.6 Hz, 1H), 7.69 (d, J=8.2 Hz, 2H), 7.46 (dd, J=9.0, 2.8 Hz, 1H), 7.31 (d, J=7.8 Hz, 2H), 5.80 (br. s., 2H), 5.72-5.87 (m, 1H).

LCMS (ES): Found 414.3 [M+H]⁺.

### Example U

### 4-(((4-(4-Fluorophenyl)pyridin-2-yl)(pyrazin-2-yl)amino)methyl)-N-hydroxybenzamide

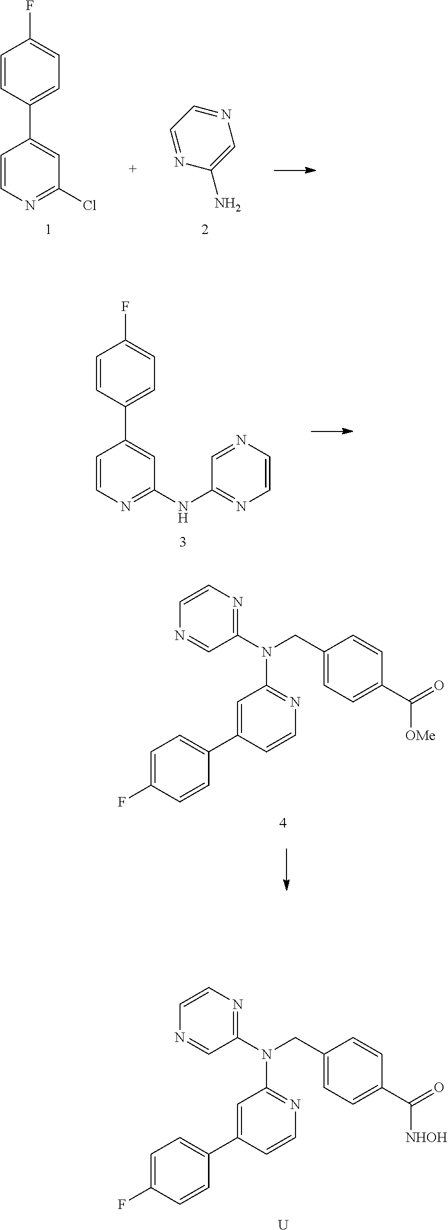

NaH (60%) (47 mg, 1.19 mmol) was added portion-wise to N-(4-(4-fluorophenyl)pyridin-2-yl)pyrazin-2-amine (3) (prepared using conditions as per Examples above) (300 mg, 1.3 mmol) in DMF (0 mL) at 5° C. under Ar(g). The reaction mixture was stirred for 20 min, then methyl 4-(bromomethyl)benzoate (337 mg, 1.47 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-(((4-(4-fluorophenyl)pyridin-2-yl)(pyrazin-2-yl)amino)methyl)benzoate (4) as a yellow solid (220 mg, 46%).

LCMS (ES): Found 414.4 [M+H]⁺.

A fresh solution of NH₂OH in MeOH was prepared: [KOH (1.49 g, 26.9 mmol) in MeOH (10 mL) was added to NH₂OH.HCl (1.86 g, 26.9 mmol) in MeOH (10 mL) at 0° C.]. The reaction mixture was stirred for 20 min at 0° C., then filtered to remove salts; it was then added to methyl 4-(((4-(4-fluorophenyl)pyridin-2-yl)(pyrazin-2-yl)amino)methyl)benzoate (4) (220 mg, 0.53 mmol) followed by KOH (298 mg, 5.3 mmol) solubilised in MeOH (10 mL). The reaction mixture was stirred at rt for 21 h and then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to 4-(((4-(4-fluorophenyl)pyridin-2-yl)(pyrazin-2-yl)amino)methyl)-N-hydroxybenzamide, Example U, as an off white solid (35 mg, 16%).

¹H NMR (400 MHz, DMSO-d₆), δ_{H} ppm: 11.10 (br. s., 1H), 8.99 (br. s., 1H), 8.69 (d, J=1.4 Hz, 1H), 8.36 (d, J=5.3 Hz, 1H), 8.28 (dd, J=2.7, 1.5 Hz, 1H), 8.11 (d, J=2.7 Hz, 1H), 7.76-7.86 (m, 2H), 7.64 (d, J=8.4 Hz, 2H), 7.42 (d, J=8.2 Hz, 2H), 7.38 (dd, J=5.3, 1.4 Hz, 1H), 7.34 (t, J=8.9 Hz, 2H), 5.53 (s, 2H).

LCMS (ES): Found 416.1 [M+H]⁺.

### Example V

### 4-((Benzo[d]thiazol-2-yl(pyridin-2-yl)amino)methyl)-N-hydroxybenzamide

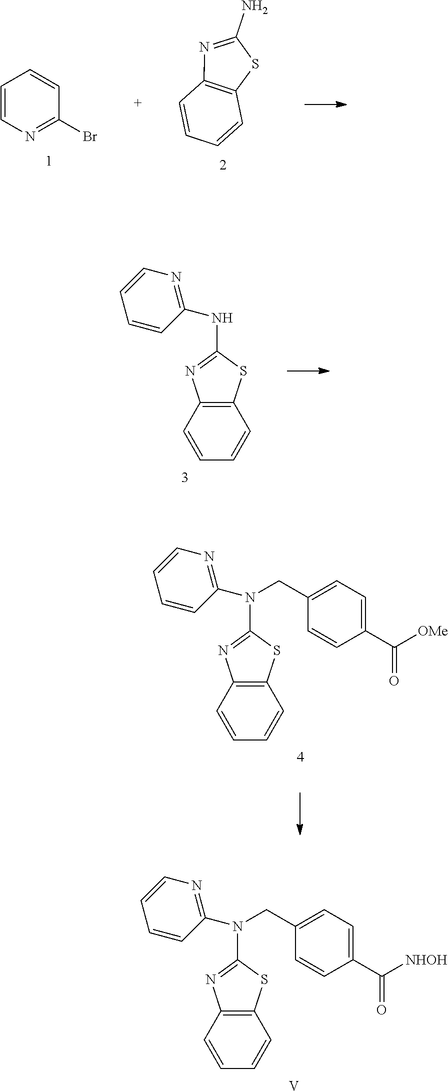

NaH (60%) (75 mg, 1.8 mmol) was added portion-wise to N-(pyridin-2-yl)benzo[d]thiazol-2-amine (3) (prepared using conditions as per Examples above) (430 mg, 1.8 mmol) in DMF (10 mL) at 5° C. under Ar(g). The reaction mixture was stirred for 20 min, then methyl 4-(bromomethyl) benzoate (563 mg, 2.4 mmol) was added and stirring was continued at 70° C. under Ar(g) for 1 h. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((benzo[d]thiazol-2-yl(pyridin-2-yl)amino)methyl)benzoate (4) as a yellow solid (300 mg, 42%).

LCMS (ES): Found 376.1 [M+H]⁺.

A fresh solution of NH₂OH in MeOH was prepared: [KOH (2.24 g, 40.0 mmol) in MeOH (15 mL) was added to NH₂OH.HCl (2.78 g, 40.0 mmol) in MeOH (15 mL) at 0° C.]. The reaction mixture was stirred for 20 min at 0° C., then filtered to remove salts; it was then added to methyl 4-((benzo[d]thiazol-2-yl(pyridin-2-yl)amino)methyl)benzoate (4) (300 mg, 0.8 mmol) followed by KOH (449 mg, 8.0 mmol) solubilised in MeOH (5 mL). The reaction mixture was stirred at rt for 21 h then concentrated in vacuo, poured onto brine/H₂O (30 mL/70 mL) and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide 4-((benzo[d]thiazol-2-yl(pyridin-2-yl)amino)methyl)-N-hydroxybenzamide, Example V, as a light orange solid (60 mg, 20%).

¹H NMR (400 MHz, DMSO-d₆), δ_{H} ppm: 11.15 (br. s, 1H), 8.99 (br. s, 1H), 8.50 (dd, J=4.8, 1.4 Hz, 1H), 7.93 (d, J=7.6 Hz, 1H), 7.78-7.86 (m, 1H), 7.68 (d, J=8.2 Hz, 2H), 7.64 (d, J=7.9 Hz, 1H), 7.33-7.39 (m, 1H), 7.21-7.31 (m, 3H), 7.11-7.20 (m, 2H), 5.82 (s, 2H).

LCMS (ES): Found 377.1 [M+H]⁺.

### Example W

### N-Hydroxy-4-((pyridin-2-yl(3-(trifluoromethyl)-1,2,4-thiadiazol-5-yl)amino)methyl)benzamide

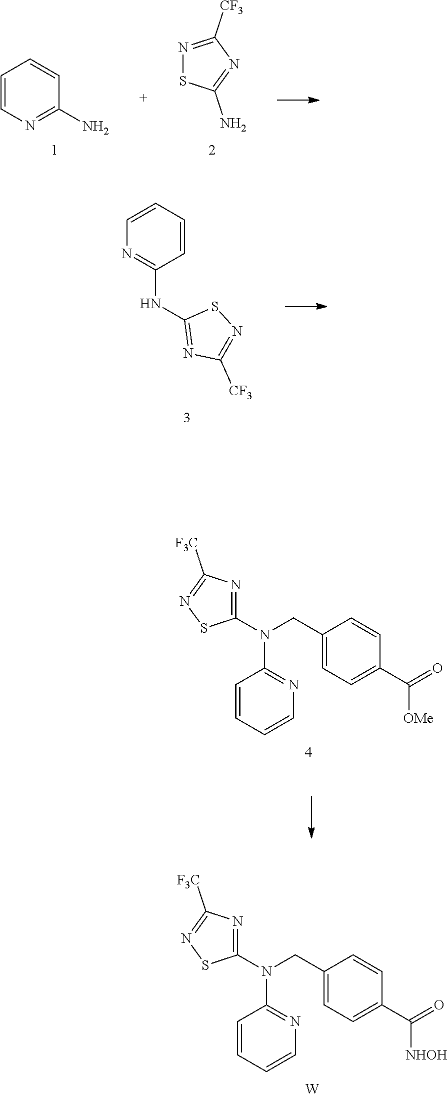

Pyridin-2-amine (1) (1.0 g, 10.6 mmol), 5-chloro-3-(trifluoromethyl)-1,2,4-thiadiazole (2) (1.82 g, 10.6 mmol), Xantphos (0.61 g, 1.06 mmol) and Cs₂CO₃ (5.18 g, 15.9 mmol) were combined in dry 1,4-dioxane (15 mL). The reaction mixture was degassed with N₂(g) and placed under vacuum for 10 min. Pd₂(dba)₃ (0.49 g, 0.53 mmol) was then added and the resulting reaction mixture was heated at 90° C. for 30 h. It was then poured onto demineralised water (200 mL) and extracted with EtOAc (3×100 mL). The organic phases were combined, dried over Na₂SO₄, filtered and subsequently concentrated in vacuo. The resulting residue was purified by flash chromatography with EtOAc/Hexane (1:1) to provide N-(pyridin-2-yl)-3-(trifluoromethyl)-1,2,4-thiadiazol-5-amine (3) as a yellow solid (1.4 g, 57%).

LCMS (ES): Found 247.2 [M+H]⁺.

NaH (60%) (49 mg, 1.21 mmol) was added portion-wise to N-(pyridin-2-yl)-3-(trifluoromethyl)-1,2,4-thiadiazol-5-amine (3) (300 mg, 1.21 mmol) in DMF (10 mL) at 5° C. under Ar(g). The reaction mixture was stirred for 20 min, then methyl 4-(bromomethyl) benzoate (363 mg, 1.58 mmol) was added and stirring was continued at 70° C. under Ar(g) for 1 h in the dark. The reaction mixture was then poured onto demineralised water (100 mL) and extracted with EtOAc (3×50 mL). The organic phases were combined, dried over Na₂SO₄, filtered and subsequently concentrated in vacuo. The resulting residue was purified by flash chromatography with EtOAc/Hexane (3:7) to furnish methyl 4-((pyridin-2-yl(3-(trifluoromethyl)-1,2,4-thiadiazol-5-yl)amino)methyl)benzoate (4) as a yellow solid (450 mg, 90%).

LCMS (ES): Found 395.3 [M+H]⁺.

A fresh solution of NH₂OH in MeOH was prepared: [KOH (3.56 g, 63.4 mmol) in MeOH (20 mL) was added to NH₂OH.HCl (4.41 g, 63.4 mmol) in MeOH (20 mL) at 0° C.]. The reaction mixture was stirred for 20 min at 0° C., then filtered to remove salts; it was then added to methyl 4-((pyridin-2-yl(3-(trifluoromethyl)-1,2,4-thiadiazol-5-yl)amino)methyl)benzoate (4) (500 mg, 1.2 mmol) followed by KOH (712 mg, 12.6 mmol) solubilised in MeOH (10 mL). The reaction mixture was stirred at rt for 21 h then concentrated in vacuo, poured onto brine/H₂O (30 mL/70 mL), and extracted with CH₂Cl₂ (3×100 mL). The organic phases were combined, dried over Na₂SO₄, filtered and subsequently concentrated in vacuo. The resulting residue was purified by flash chromatography with MeOH/CH₂Cl₂ (1:9) to provide N-hydroxy-4-((pyridin-2-yl(3-(trifluoromethyl)-1,2,4-thiadiazol-5-yl)amino)methyl)benzamide, Example W, as an off white solid (20 mg, 4%).

¹H NMR (400 MHz, DMSO-d₆), δ_{H} ppm: 11.15 (br. s., 1H), 9.03 (br. s., 1H), 8.63-8.68 (m, J=5.0, 0.9 Hz, 1H), 7.97 (ddd, J=8.7, 7.2, 1.8 Hz, 1H), 7.69 (d, J=8.4 Hz, 2H), 7.41 (d, J=8.6 Hz, 1H), 7.32 (d, J=8.3 Hz, 2H), 7.28 (dd, J=7.0, 5.3 Hz, 1H), 5.80 (s, 2H).

LCMS (ES): Found 396.3 [M+H]⁺.

### Example X

### N-Hydroxy-4-(((3-methoxypyridin-2-yl)-(5-methylpyridin-2-yl)amino)methyl)benzamide

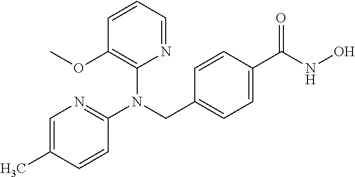 [SMILES: COc1cccnc1N(Cc1ccc(C(=O)NO)cc1)c1ccc(C)cn1]

¹H NMR (400 MHz, Methanol-d₄), δ_{H} ppm: 7.97 (d, J=4.9 Hz, 1H), 7.89 (d, J=2.3 Hz, 1H), 7.61 (d, J=7.8 Hz, 2H), 7.46 (m, 3H), 7.33 (dd, J=8.5, 2.4 Hz, 1H), 7.22 (dd, J=8.2, 4.8 Hz, 1H), 6.41 (d, J=8.5 Hz, 1H), 5.31 (s, 2H), 3.73 (s, 3H), 2.20 (s, 3H).

LCMS (ES): Found 365.0 [M+H]⁺.

### Example Y

### N-Hydroxy-4-(((5-methoxypyridin-2-yl)(5-methylpyridin-2-yl)amino)methyl)benzamide

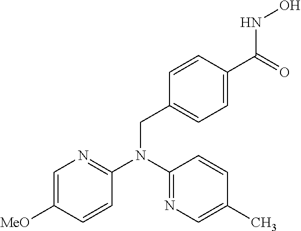 [SMILES: COc1ccc(N(Cc2ccc(C(=O)NO)cc2)c2ccc(C)cn2)nc1]

¹H NMR (400 MHz, Methanol-d₄), δ_{H} ppm: 7.99 (dd, J=4.8, 2.6 Hz, 2H), 7.62 (d, J=8.0 Hz, 2H), 7.41 (m, 3H), 7.31 (dd, J=9.1, 3.1 Hz, 1H), 7.14 (d, J=8.9 Hz, 1H), 6.84 (d, J=8.5 Hz, 1H), 5.36 (s, 2H), 3.83 (s, 3H), 2.22 (s, 3H).

LCMS (ES): Found 365.0 [M+H]⁺.

### Example Z

### N-Hydroxy-4-(((3-methoxypyridin-2-yl)(5-morpholinopyridin-2-yl)amino)methyl)benzamide

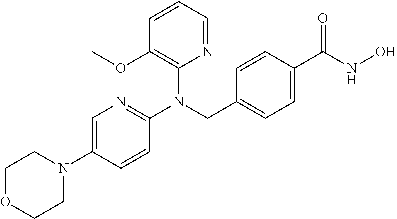 [SMILES: COc1cccnc1N(Cc1ccc(C(=O)NO)cc1)c1ccc(N2CCOCC2)cn1]

¹H NMR (400 MHz, Methanol-d₄), δ_{H} ppm: 7.94 (dd, J=4.8, 1.5 Hz, 1H), 7.78 (d, J=3.0 Hz, 1H), 7.61 (d, J=8.3 Hz, 2H), 7.38-7.51 (m, 3H), 7.27 (dd, J=9.0, 3.1 Hz, 1H), 7.17 (dd, J=8.1, 4.8 Hz, 1H), 6.51 (d, J=9.0 Hz, 1H), 5.31 (s, 2H), 3.77-3.89 (m, 4H), 3.72 (s, 3H), 2.97-3.08 (m, 4H).

LCMS (ES): Found 436.0 [M+H]⁺.

### Example AA

### N-Hydroxy-4-(((5-methoxypyridin-2-yl)(5-morpholinopyridin-2-yl)amino)methyl)benzamide

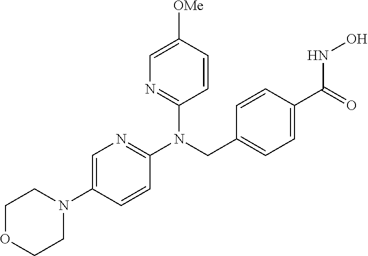 [SMILES: COc1ccc(N(Cc2ccc(C(=O)NO)cc2)c2ccc(N3CCOCC3)cn2)nc1]

¹H NMR (400 MHz, Methanol-d₄), δ_{H} ppm: 7.88-7.95 (m, 2H), 7.58-7.66 (m, 2H), 7.42 (d, J=8.0 Hz, 2H), 7.33 (dd, J=9.0, 3.1 Hz, 1H), 7.26 (dd, J=9.1, 3.1 Hz, 1H), 6.99 (dd, J=9.0, 4.5 Hz, 2H), 5.34 (s, 2H), 3.71-3.94 (m, 7H), 3.04-3.15 (m, 4H).

LCMS (ES): Found 436.0 [M+H]⁺.

### Example BB

### N-Hydroxy-4-((pyridin-2-yl(thieno[3,2-c]pyridin-4-yl)amino)methyl)benzamide

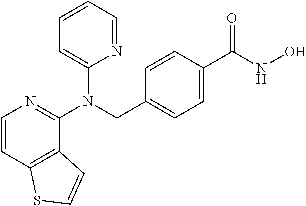 [SMILES: O=C(NO)c1ccc(CN(c2ccccn2)c2nccc3sccc23)cc1]

¹H NMR (400 MHz, Methanol-d₄), δ_{H} ppm: 7.97-8.10 (m, 1H), 7.76 (dd, J=9.3, 7.1 Hz, 3H), 7.33-7.69 (m, 5H), 7.14 (d, J=5.4 Hz, 1H), 6.98 (d, J=9.1 Hz, 1H), 6.64 (t, J=6.8 Hz, 1H), 5.56 (s, 2H).

LCMS (ES): Found 377.0 [M+H]⁺.

### Example CC

### N-Hydroxy-4-(((6-methylpyridin-2-yl)(5-morpholinopyridin-2-yl)amino)methyl)benzamide

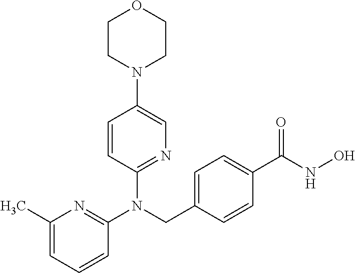 [SMILES: Cc1cccc(N(Cc2ccc(C(=O)NO)cc2)c2ccc(N3CCOCC3)cn2)n1]

¹H NMR (400 MHz, Methanol-d₄), δ_{H} ppm: 7.99 (d, J=3.0 Hz, 1H), 7.62 (d, J=7.8 Hz, 2H), 7.42 (d, J=8.1 Hz, 2H), 7.34-7.39 (m, 2H), 7.14 (d, J=8.9 Hz, 1H), 6.64 (dd, J=8.1, 7.8 Hz, 2H), 5.39 (s, 2H), 3.79-3.86 (m, 4H), 3.14 (dd, J=6.1, 3.6 Hz, 4H), 2.37 (s, 3H).

LCMS (ES): Found 420.0 [M+H]⁺.

### Example DD

### N-Hydroxy-4-{[(pyrazin-2-yl)(pyrimidin-4-yl)amino]methyl}benzamide

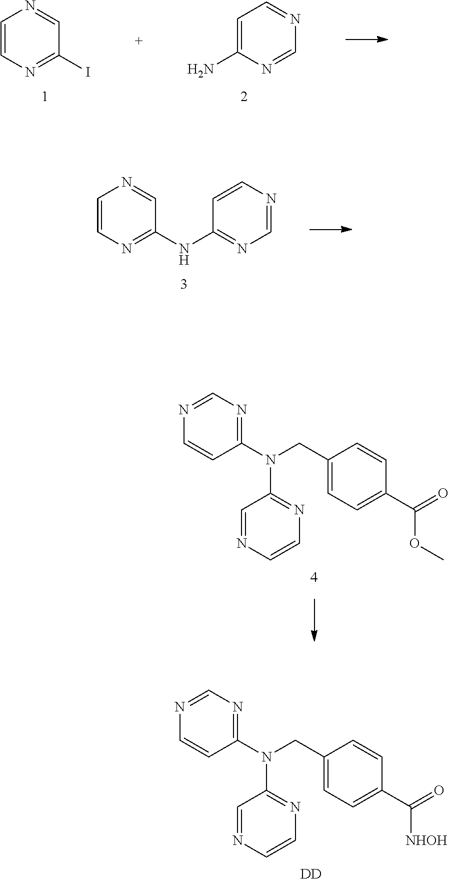

A solution of 2-iodopyrazine (1) (1.2 g, 5.83 mmol), pyrimidin-4-amine (2) (609 mg, 6.41 mmol), Cs₂CO₃ (3.80 g, 11.65 mmol) and Xantphos (148 mg, 0.26 mmol) in 1,4-dioxane (15 mL) was purged with N₂(g) for 10 min. Pd₂(dba)₃ (107 mg, 0.12 mmol) was added and mixture was heated to 90° C. for 3 h. Reaction was cooled to rt and partitioned between water (300 mL) and EtOAc (3×100 mL). Combined organics were washed with water (50 mL), dried over Na₂SO₄, filtered and concentrated in vacuo. The residue was purified by flash column chromatography with CH₂Cl₂/MeOH (1:0-9:1) to yield (3) (678 mg, 66%).

¹H NMR (500 MHz, Methanol-d₄), δ_{H} ppm: 9.06 (d, J=1.3 Hz, 1H), 8.74 (s, 1H), 8.42 (d, J=6.0 Hz, 1H), 8.34 (dd, J=2.6, 1.5 Hz, 1H), 8.19 (d, J=2.7 Hz, 1H), 7.72 (dd, J=6.0, 1.0 Hz, 1H).

LCMS (ES): Found 174.0 [M+H]⁺.

NaH (60%, 48.5 mg, 1.21 mmol) was added to a solution of (3) (200 mg, 1.15 mmol) in DMF (7 mL) at 5° C. under N₂(g). The reaction mixture was stirred for 20 min then methyl 4-(bromomethyl)benzoate (344 mg, 1.5 mmol) was added as a solution in DMF (3 mL), the stirring was continued at 70° C. for 1 h. Reaction cooled to rt and poured onto water (100 mL). Brine (25 mL) was added and the aqueous layer was extracted with EtOAc (2×100 mL). Combined organics were dried over Na₂SO₄, filtered and concentrated in vacuo. Purification by flash column chromatography with CH₂Cl₂/EtOAc (1:0-0:1) then EtOAc/MeOH (1:0-4:1) yielded (4) (187 mg, 50%).

¹H NMR (500 MHz, Chloroform-d), δ_{H} ppm: 8.85 (d, J=1.4 Hz, 1H), 8.77-8.80 (m, 1H), 8.34-8.38 (m, 2H), 8.29 (d, J=2.6 Hz, 1H), 7.95 (d, J=8.4 Hz, 2H), 7.36 (d, J=8.4 Hz, 2H), 6.91 (dd, J=6.0, 1.2 Hz, 1H), 5.49 (s, 2H), 3.87 (s, 3H).

LCMS (ES): Found 322.0 [M+H]⁺.

A solution of (4) (0.09 mL, 0.58 mmol) in 0.85M hydroxylamine in MeOH (10 mL) was stirred at rt for 40 h. Solvent was removed in vacuo and the residue purified by reverse phase HPLC to give Example DD (30 mg, 15%).

¹H NMR (500 MHz, Methanol-d₄), δ_{H} ppm: 8.89 (d, J=1.4 Hz, 1H), 8.69 (s, 1H), 8.47 (dd, J=2.5, 1.5 Hz, 1H), 8.25-8.37 (m, 2H), 7.68 (d, J=8.3 Hz, 2H), 7.38 (d, J=8.3 Hz, 2H), 7.08 (dd, J=6.2, 1.2 Hz, 1H), 5.51 (s, 2H).

LCMS (ES): Found 323.0 [M+H]⁺.

### Example EE

### N-Hydroxy-4-{[(pyrazin-2-yl)(pyrimidin-4-yl)amino]methyl}benzamide

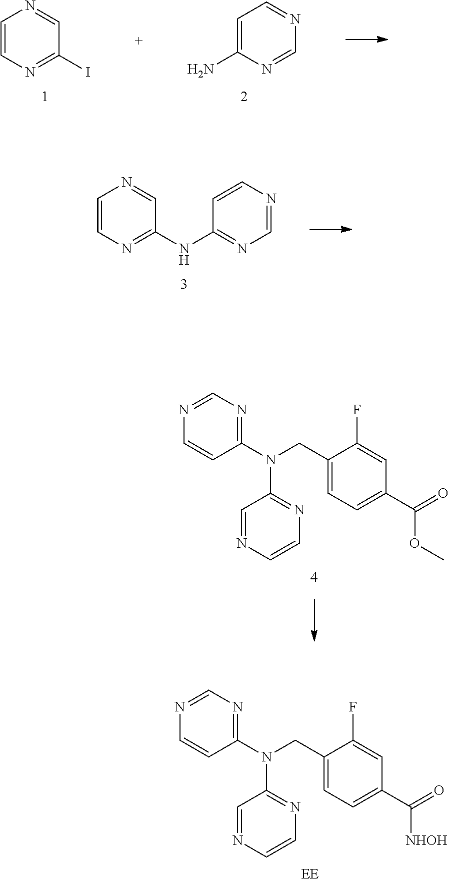

NaH (60%, 48.5 mg, 1.21 mmol) was added to a solution of (3) (200 mg, 1.15 mmol) in DMF (7 mL) at 5° C. under N₂(g). The reaction mixture was stirred for 20 min then methyl 4-(bromomethyl)-3-fluorobenzoate (371 mg, 1.5 mmol) was added as a solution in DMF (3 mL). The stirring was continued at 70° C. for 1 h. Reaction cooled to rt and poured onto water (100 mL). Brine (25 mL) was added and the aqueous layer was extracted with EtOAc (2×100 mL). Combined organics were dried over Na₂SO₄, filtered and concentrated in vacuo. Purification by flash column chromatography with EtOAc/CH₂Cl₂ (0:1-1:0) then EtOAc/MeOH (1:0-4:1) yielded (4) (158 mg, 40%).

¹H NMR (500 MHz, Chloroform-d), δ_{H} ppm: 8.87 (d, J=1.4 Hz, 1H), 8.76-8.78 (m, 1H), 8.36-8.40 (m, 2H), 8.31 (d, J=2.6 Hz, 1H), 7.69 (d, J=9.2 Hz, 2H), 7.30 (t, J=7.6 Hz, 1H), 6.92 (dd, J=6.1, 1.2 Hz, 1H), 5.50 (s, 2H), 3.87 (s, 3H).

LCMS (ES): Found 340.0 [M+H]⁺.

A solution of (4) (0.08 mL, 0.47 mmol) in 0.85M hydroxylamine in MeOH (10 mL) was stirred at rt for 18 h. Solvent was concentrated to dryness and the residue purified by neutral pH reverse phase HPLC to give Example EE (25 mg, 15%).

¹H NMR (500 MHz, Methanol-d₄), δ_{H} ppm: 8.91 (d, J=1.4 Hz, 1H), 8.70 (s, 1H), 8.48 (dd, J=2.5, 1.5 Hz, 1H), 8.31-8.38 (m, 2H), 7.43-7.50 (m, 2H), 7.35 (t, J=7.9 Hz, 1H), 7.09 (dd, J=6.2, 1.2 Hz, 1H), 5.53 (s, 2H).

LCMS (ES): Found 341.0 [M+H]⁺.

### Example FF

### N-Hydroxy-6-{[(pyrazin-2-yl)(pyrimidin-4-yl)amino]methyl}pyridine-3-carboxamide

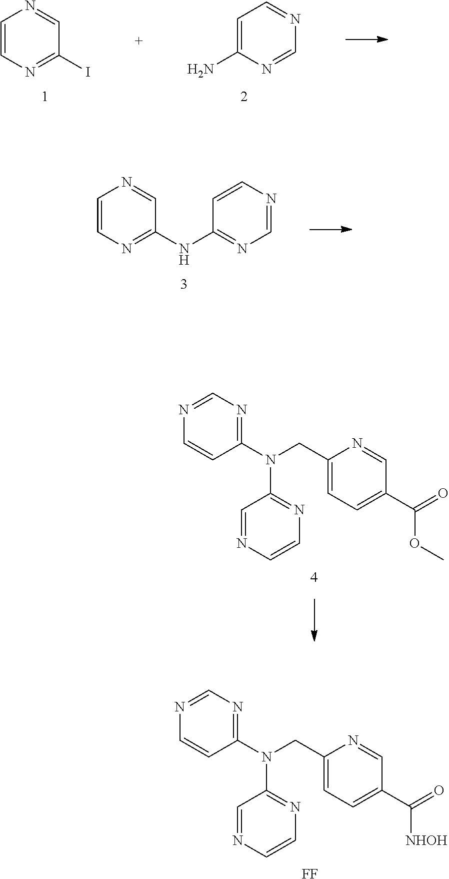

NaH (60%, 48.5 mg, 1.21 mmol) was added to a solution of (3) (200 mg, 1.15 mmol) in DMF (7 mL) at 5° C. under N₂(g). The reaction mixture was stirred for 20 min then methyl 6-(bromomethyl)pyridine-3-carboxylate (345 mg, 1.5 mmol) was added as a solution in DMF (3 mL). The stirring was continued at 70° C. for 1 h. Reaction cooled to rt and poured onto water (100 mL). Brine (25 mL) was added and the aqueous layer was extracted with EtOAc (2×100 mL). Combined organics were dried over Na₂SO₄, filtered and concentrated in vacuo. The residue was purified by flash column chromatography with CH₂Cl₂/EtOAc (1:0-0:1) then CH₂Cl₂/MeOH (1:0-4:1) to yield (4) (116 mg, 27%).

¹H NMR (500 MHz, Chloroform-d), δ_{H} ppm: 9.11 (d, J=1.6 Hz, 1H), 8.97 (d, J=1.4 Hz, 1H), 8.70-8.77 (m, 1H), 8.34-8.40 (m, 2H), 8.31 (d, J=2.6 Hz, 1H), 8.18 (dd, J=8.2, 2.1 Hz, 1H), 7.36 (d, J=8.2 Hz, 1H), 7.01 (dd, J=6.1, 1.2 Hz, 1H), 5.56 (s, 2H), 3.90 (s, 3H).

LCMS (ES): Found 322.9 [M+H]⁺.

A solution of (4) (0.06 mL, 0.31 mmol) in 0.85M hydroxylamine in MeOH (10 mL) was stirred at rt for 18 h. The reaction mixture was concentrated to dryness. The residue was purified by reverse phase HPLC to give Example FF (25.7 mg, 26%).

¹H NMR (500 MHz, DMSO-d₆), δ_{H} ppm: 8.99 (d, J=4.9 Hz, 1H), 8.64-8.76 (m, 2H), 8.32-8.51 (m, 3H), 7.82-7.93 (m, 1H), 7.03-7.30 (m, 2H), 5.45 (m, 2H).

LCMS (ES): Found 324.1 [M+H]⁺.

### Example GG

### 4-{[Bis(pyrazin-2-yl)amino]methyl}-N-hydroxybenzamide

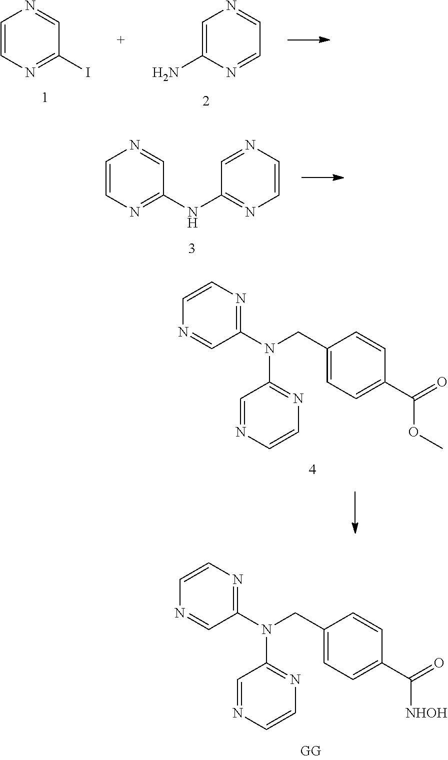

A solution of 2-iodopyrazine (1) (1.2 g, 5.83 mmol), pyrazin-2-amine (2) (609 mg, 6.4 mmol), Cs₂CO₃ (3.80 g, 11.7 mmol) and Xantphos (148 mg, 0.26 mmol) in dioxane (25 mL) was purged with N₂(g) for 10 min. Pd₂(dba)₃ (107 mg, 0.12 mmol) was added and mixture was heated to 90° C. for 3 h. Reaction cooled to rt and poured onto water (200 mL), extracted with EtOAc (2×150 mL) and CH₂Cl₂-IPA (150 mL, 4:1). Combined organics were dried over Na₂SO₄, filtered and concentrated in vacuo. Flash column chromatography with heptane/EtOAc (4:1-0:1) then EtOAc/MeOH (1:0-3:1) yielded (3) as an off white solid (210 mg, 51%).

¹H NMR (500 MHz, Chloroform-d), δ_{H} ppm: 8.99 (d, J=1.4 Hz, 2H), 8.30 (dd, J=2.6, 1.5 Hz, 2H), 8.11 (d, J=2.7 Hz, 2H).

LCMS (ES): Found 174.1 [M+H]⁺.

NaH (60%, 48.5 mg, 1.21 mmol) was added to a solution of (3) (200 mg, 1.15 mmol) in DMF (7 mL) at 5° C. under N₂(g). The reaction mixture was stirred for 20 min then methyl 4-(bromomethyl)benzoate (344 mg, 1.5 mmol) was added as a solution in DMF (3 mL). The stirring was continued at 70° C. for 1 h. Reaction cooled to rt and poured onto water (100 mL). Brine (25 mL) was added and extracted with EtOAc (2×100 mL). Combined organic was dried over Na₂SO₄, filtered and concentrated in vacuo. The residue was purified by flash column chromatography with CH₂Cl₂/EtOAc (1:0-0:1) then EtOAc/MeOH (1:0-4:1) to give (4) (196 mg, 53%).

¹H NMR (500 MHz, Chloroform-d), δ_{H} ppm: 8.59-8.65 (m, 2H), 8.23-8.26 (m, 2H), 8.16 (d, J=2.5 Hz, 2H), 7.94 (d, J=8.3 Hz, 2H), 7.38 (d, J=8.2 Hz, 2H), 5.50 (s, 2H), 3.86 (s, 3H).

LCMS (ES): Found 321.9 [M+H]⁺.

A solution of (4) (0.09 mL, 0.61 mmol) in 0.85M hydroxylamine in MeOH (10 mL) was stirred at rt for 72 h. Solvent concentrated to dryness and the residue purified by reverse phase HPLC to give Example GG (23 mg, 12%).

¹H NMR (500 MHz, Methanol-d₄), δ_{H} ppm: 8.66 (d, J=1.3 Hz, 2H), 8.28-8.36 (m, 2H), 8.16 (d, J=2.6 Hz, 2H), 7.67 (d, J=8.2 Hz, 2H), 7.45 (d, J=8.2 Hz, 2H), 5.56 (s, 2H).

LCMS (ES): Found 323.1 [M+H]⁺.

### Example HH

### 4-{[Bis(pyrazin-2-yl)amino]methyl}-3-fluoro-N-hydroxybenzamide

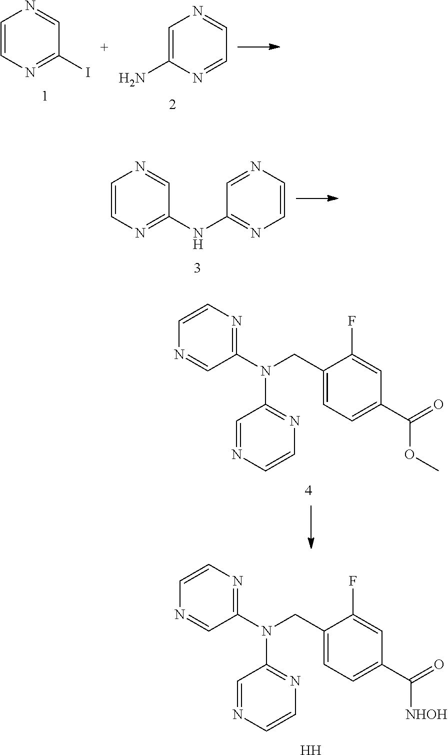

NaH (60%, 49 mg, 1.21 mmol) was added to a solution of (3) (200 mg, 1.15 mmol) in DMF (7 mL) at 5° C. under N₂(g). The reaction mixture was stirred for 20 min then methyl 4-(bromomethyl)-3-fluorobenzoate (371 mg, 1.5 mmol) was added as a solution in DMF (3 mL). The stirring was continued at 70° C. for 1 h. Reaction cooled to rt and poured onto water (100 mL). Brine (25 mL) was added and the aqueous was extracted with EtOAc (2×100 mL). Combined organics were dried over Na₂SO₄, filtered and concentrated in vacuo. Purification by flash column chromatography with CH₂Cl₂/EtOAc (1:0-0:1) then EtOAc/MeOH (1:0-4:1) yielded (4) (195 mg, 50%).

¹H NMR (500 MHz, Chloroform-d), δ_{H} ppm: 8.65 (d, J=1.4 Hz, 2H), 8.25 (dd, J=2.5, 1.5 Hz, 2H), 8.18 (d, J=2.6 Hz, 2H), 7.65-7.72 (m, 2H), 7.31 (t, J=7.8 Hz, 1H), 5.53 (s, 2H), 3.87 (s, 3H).

LCMS (ES): Found 339.9 [M+H]⁺.

A solution of (4) (0.09 mL, 0.57 mmol) in 0.85M hydroxylamine in MeOH (10 mL) was stirred at rt for 18 h. Solvent was concentrated in vacuo and the residue purified by reverse phase HPLC to give Example HH (81 mg, 41%).

¹H NMR (500 MHz, DMSO-d₆), δ_{H} ppm: 8.76 (d, J=1.4 Hz, 2H), 8.34 (dd, J=2.5, 1.5 Hz, 2H), 8.25 (d, J=2.6 Hz, 2H), 7.51 (dd, J=11.1, 1.3 Hz, 1H), 7.45 (dd, J=8.0, 1.4 Hz, 1H), 7.34 (t, J=7.8 Hz, 1H), 5.50 (s, 2H).

LCMS (ES): Found 341.1 [M+H]⁺.

### Example II

### 6-{[Bis(pyrazin-2-yl)amino]methyl}-N-hydroxypyridine-3-carboxamide

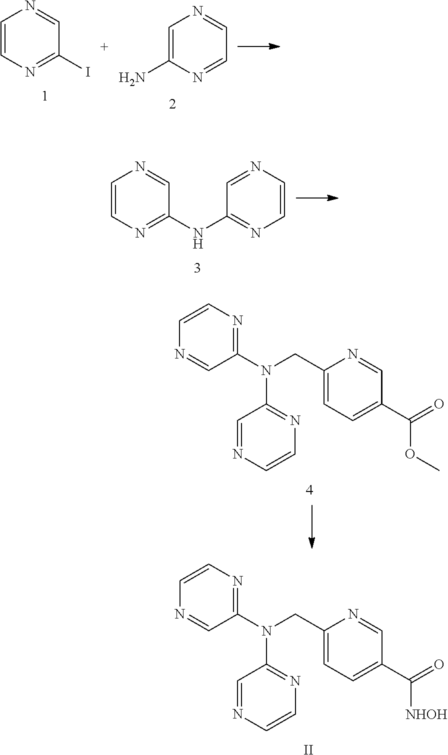

NaH (60%, 48.5 mg, 1.21 mmol) was added to a solution of (3) (200 mg, 1.15 mmol) in DMF (7 mL) at 5° C. under N₂(g). The reaction mixture was stirred for 20 min then methyl 6-(bromomethyl)pyridine-3-carboxylate (345 mg, 1.5 mmol) was added as a solution in DMF (3 mL). The stirring was continued at 70° C. for 1 h. Reaction cooled to rt and poured onto water (100 mL). Brine (25 mL) was added and the aqueous was extracted with EtOAc (2×100 mL). Combined organics were dried over Na₂SO₄, filtered and concentrated in vacuo. The residue was purified by flash column chromatography with CH₂Cl₂/EtOAc (1:0-0:1) then EtOAc/MeOH (1:0-4:1) to give (4) (129 mg, 35%).

¹H NMR (500 MHz, Chloroform-d), δ_{H} ppm: 9.04-9.13 (m, 1H), 8.70 (s, 2H), 8.19 (s, 2H), 8.13 (dd, J=5.6, 2.3 Hz, 3H), 7.32 (d, J=8.2 Hz, 1H), 5.55 (s, 2H), 3.86 (s, 3H).

LCMS (ES): Found 322.9 [M+H]⁺.

A solution of (4) (0.06 mL, 0.4 mmol) in 0.85M hydroxylamine in MeOH (10 mL) was stirred at rt for 18 h. The solvent was concentrated to dryness and the residue purified by reverse phase HPLC to give Example II (37 mg, 28%).

¹H NMR (500 MHz, DMSO-d₆), δ_{H} ppm: 8.75 (d, J=1.3 Hz, 3H), 8.31 (dd, J=2.6, 1.5 Hz, 2H), 8.21 (d, J=2.6 Hz, 2H), 7.89 (dd, J=8.1, 2.0 Hz, 1H), 7.18 (d, J=8.1 Hz, 1H), 5.47 (s, 2H).

LCMS (ES): Found 324.1 [M+H]⁺.

### Example JJ

### N-Hydroxy-4-{[(3-methoxypyridin-2-yl)pyrazin-2-yl)amino]methyl}benzamide

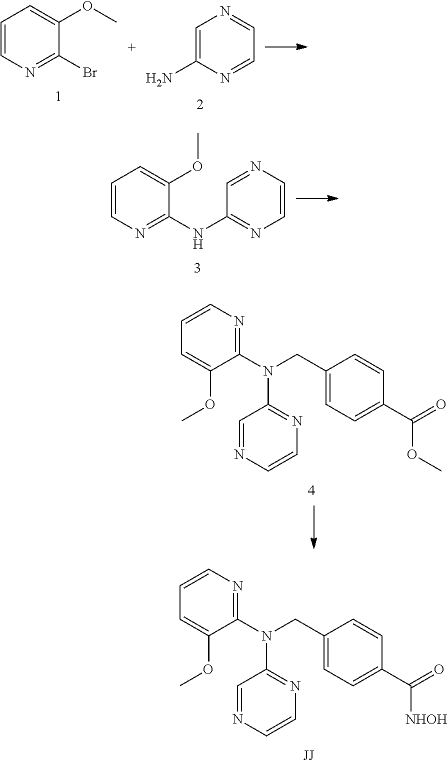

A solution of pyrazin-2-amine (2) (557 mg, 5.85 mmol), 2-bromo-3-methoxypyridine (1) (1.0 g, 5.32 mmol), Cs₂CO₃ (3.47 g, 10.64 mmol) and Xantphos (135 mg, 0.23 mmol) in dioxane (15 mL) was purged with N₂(g) for 10 min. Pd₂(dba)₃ (97.4 mg, 0.11 mmol) was added and the mixture was heated to 90° C. for 3 h. The reaction was cooled to rt, partitioned between water (200 mL) and EtOAc (200 mL). Phases were separated and aqueous layer was washed with EtOAc (200+100+50 mL). Combined organics were dried over Na₂SO₄, filtered and concentrated in vacuo. The residue was purified by flash column chromatography eluted with a gradient of CH₂Cl₂/EtOAc (1:0-0:1) to yield (3) (1.0 g, 88%).

¹H NMR (500 MHz, Chloroform-d), δ_{H} ppm: 9.91 (d, J=1.2 Hz, 1H), 8.11-8.20 (m, 2H), 7.91 (dd, J=5.0, 1.4 Hz, 1H), 7.80 (s, 1H), 7.06 (dd, J=7.9, 1.3 Hz, 1H), 6.85 (dd, J=7.9, 5.0 Hz, 1H), 3.92 (s, 3H).

LCMS (ES): Found 203.2 [M+H]⁺.

NaH (60%, 41.5 mg, 1.04 mmol) was added to a solution of (3) (200 mg, 0.99 mmol) in DMF (10 mL) at 5° C. under N₂(g). The reaction mixture was stirred for 20 min then methyl 4-(bromomethyl)benzoate (294 mg, 1.29 mmol) was added. The stirring was continued at 70° C. under N₂(g) for 1 h. The reaction was cooled to rt and poured onto water (150 mL) and brine (50 mL), the aqueous layer was extracted with EtOAc (3×100 mL). Combined organics were dried over Na₂SO₄, filtered and concentrated in vacuo. The residue was purified by flash column chromatography with CH₂Cl₂/EtOAc (1:0-0:1) then EtOAc/MeOH (1:0-4:1) to yield (4) (251 mg, 73%).

¹H NMR (500 MHz, Chloroform-d), δ_{H} ppm: 8.06-8.10 (m, 2H), 7.87-7.92 (m, 3H), 7.78 (d, J=1.5 Hz, 1H), 7.44 (d, J=8.4 Hz, 2H), 7.23 (dd, J=8.2, 1.4 Hz, 1H), 7.15 (dd, J=8.1, 4.7 Hz, 1H), 5.42 (s, 2H), 3.85 (s, 3H), 3.73 (s, 3H).

LCMS (ES): Found 350.9 [M+H]⁺.

A solution of (4) (251 mg, 0.72 mmol) in 0.85M hydroxylamine in MeOH (10 mL) was stirred at rt for 72 h. The solvent concentrated to dryness and the residue purified by reverse HPLC to give Example JJ (101 mg, 40%) as a beige solid.

¹H NMR (500 MHz, DMSO-d₆), δ_{H} ppm: 8.11 (dd, J=2.6, 1.6 Hz, 1H), 8.07 (dd, J=4.7, 1.3 Hz, 1H), 7.93 (d, J=2.7 Hz, 1H), 7.79 (d, J=1.4 Hz, 1H), 7.61 (d, J=8.2 Hz, 2H), 7.58 (dd, J=8.2, 1.2 Hz, 1H), 7.38 (d, J=8.2 Hz, 2H), 7.32 (dd, J=8.2, 4.7 Hz, 1H), 5.30 (s, 2H), 3.76 (s, 3H).

LCMS (ES): Found 352.1 [M+H]⁺.

### Example KK

### 3-Fluoro-N-hydroxy-4-{[(3-methoxypyridin-2-yl)(pyrazin-2-yl)amino]methyl}benzamide

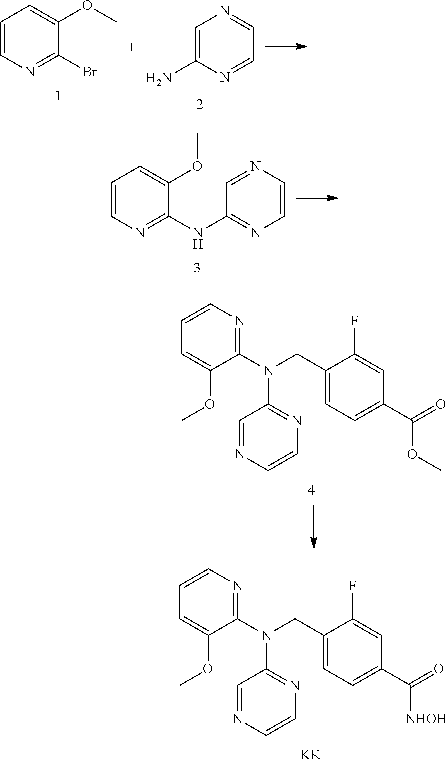

NaH (60%, 41.5 mg, 1.04 mmol) was added to a solution of (3) (200 mg, 0.99 mmol) in DMF (10 mL) at 5° C. under N₂(g). The reaction mixture was stirred for 20 min then methyl 4-(bromomethyl)-3-fluorobenzoate (318 mg, 1.29 mmol) was added. The stirring was continued at 70° C. under N₂(g) for 1 h. The reaction cooled to rt and poured onto water (150 mL) and brine (50 mL), the aqueous layer extracted with EtOAc (3×100 mL). Combined organics were dried over Na₂SO₄, filtered and concentrated in vacuo. The residue was purified by flash column chromatography with CH₂Cl₂/EtOAc (1:0-0:1) then EtOAc/MeOH (1:0-4:1) to give (4) (269 mg, 74%).

¹H NMR (500 MHz, Chloroform-d), δ_{H} ppm: 8.09 (dd, J=4.7, 1.4 Hz, 1H), 8.06 (dd, J=2.6, 1.6 Hz, 1H), 7.90 (d, J=2.7 Hz, 1H), 7.80 (d, J=1.3 Hz, 1H), 7.68 (dd, J=8.0, 1.4 Hz, 1H), 7.62 (dd, J=10.5, 1.4 Hz, 1H), 7.56 (t, J=7.7 Hz, 1H), 7.27 (dd, J=8.3, 1.5 Hz, 1H), 7.18 (dd, J=8.2, 4.7 Hz, 1H), 5.43 (s, 2H), 3.86 (s, 3H), 3.77 (s, 3H).

LCMS (ES): Found 368.9 [M+H]⁺.

A solution of (4) (269 mg, 0.73 mmol) in 0.85M hydroxylamine in MeOH (10 mL) was stirred at rt for 72 h. The solvent was concentrated to dryness and the residue purified by reverse phase HPLC to give Example KK (93 mg, 35%).

¹H NMR (500 MHz, DMSO-d₆), δ_{H} ppm: 8.13 (dd, J=2.6, 1.6 Hz, 1H), 8.08 (dd, J=4.7, 1.3 Hz, 1H), 7.95 (d, J=2.7 Hz, 1H), 7.80 (d, J=1.3 Hz, 1H), 7.61 (dd, J=8.3, 1.2 Hz, 1H), 7.48-7.43 (m, 3H), 7.35 (dd, J=8.2, 4.7 Hz, 1H), 5.32 (s, 2H), 3.78 (s, 3H).

LCMS (ES): Found 370.1 [M+H]⁺.

### Example LL

### N-Hydroxy-6-{[(3-methoxypyridin-2-yl)(pyrazin-2-yl)amino]methyl}pyridine-3-carboxamide

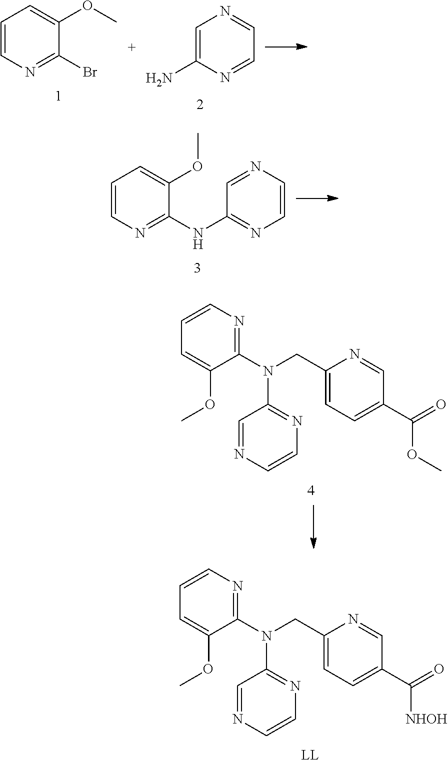

NaH (60%, 41.5 mg, 1.04 mmol) was added to a solution of (3) (200 mg, 0.99 mmol) in DMF (10 mL) at 5° C. under N₂(g). The reaction mixture was stirred for 20 min then methyl 6-(bromomethyl)pyridine-3-carboxylate (296 mg, 1.29 mmol) was added. The stirring was continued at 70° C. under N₂(g) for 1 h. The reaction was cooled to rt and poured onto water (150 mL) and brine (50 mL), then the aqueous layer was extracted with EtOAc (3×100 mL). Combined organics were dried over Na₂SO₄, filtered and concentrated in vacuo. The residue was purified by flash column chromatography with CH₂Cl₂/EtOAc (1:0-0:1) then EtOAc/MeOH (1:0-4:1) to give (4) (191 mg, 55%).

¹H NMR (500 MHz, Chloroform-d), δ_{H} ppm: 9.07 (d, J=1.9 Hz, 1H), 8.12 (dd, J=8.2, 2.1 Hz, 1H), 8.06 (dd, J=4.7, 1.4 Hz, 1H), 8.01 (dd, J=2.6, 1.6 Hz, 1H), 7.88 (d, J=2.7 Hz, 1H), 7.84 (d, J=1.4 Hz, 1H), 7.54 (d, J=8.2 Hz, 1H), 7.27 (dd, J=8.2, 1.4 Hz, 1H), 7.17 (dd, J=8.2, 4.7 Hz, 1H), 5.46 (s, 2H), 3.86 (s, 3H), 3.76 (s, 3H).

LCMS (ES): Found 352.0 [M+H]⁺.

A solution of (4) (191 mg, 0.54 mmol) in 0.85M hydroxylamine in MeOH (10 mL) was stirred at rt for 72 h. After this time, the solvent was concentrated to dryness and the residue purified by reverse phase HPLC to give Example LL (35 mg, 19%).

¹H NMR (500 MHz, DMSO-d₆), δ_{H} ppm: 8.72 (d, J=1.8 Hz, 1H), 8.12-8.08 (m, 1H), 8.06 (dd, J=4.7, 1.3 Hz, 1H), 7.93 (d, J=2.7 Hz, 1H), 7.81-7.87 (m, 2H), 7.56-7.61 (m, 1H), 7.32 (dd, J=8.2, 4.7 Hz, 1H), 7.25 (d, J=8.1 Hz, 1H), 5.29 (s, 2H), 3.77 (s, 3H).

LCMS (ES): Found 353.1 [M+H]⁺.

### Example MM

### N-Hydroxy-4-{[(pyrazin-2-yl)(pyridazin-3-yl)amino]methyl}benzamide

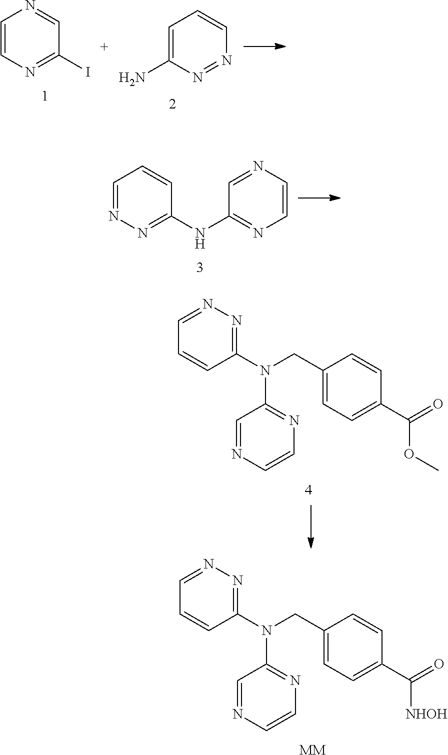

A solution of 2-iodopyrazine (1) (2.40 g, 11.65 mmol), pyridazin-3-amine (2) (1.2 g, 12.82 mmol), Cs₂CO₃ (7.6 g, 23.3 mmol) and Xantphos (297 mg, 0.51 mmol) in dioxane (45 mL) was purged with N₂(g) for 10 min. Pd₂(dba)₃ (214 mg, 0.23 mmol) in dioxane (5 mL) was added and the mixture was heated to 90° C. for 3 h. The reaction was cooled to rt and partitioned between water (200 mL) and EtOAc (200 mL). The insoluble solid was filtered and put a-side. The phases were separated and the aqueous layer was extracted with EtOAc (200 mL), then CH₂Cl₂-IPA (200 mL, 4:1). Combined organics were dried over Na₂SO₄, filtered and concentrated in vacuo. The residue was purified by flash column chromatography with CH₂Cl₂/EtOAc (1:0-0:1) then EtOAc/MeOH (1:0-4:1) to yield (3). The solid [from filtration] was washed with water (100 mL) and triturated with hot MeOH (3×100 mL) and filtered. The filtrates were concentrated to yield a second batch of (3). The solid was further washed with water (100 mL) and was sucked dry to yield a third batch of (3). All three batches were combined to give (3) (1.63 g, 80%).

¹H NMR (500 MHz, DMSO-d₆), δ_{H} ppm: 10.49 (s, 1H), 9.00 (d, J=1.2 Hz, 1H), 8.83 (dd, J=4.6, 1.2 Hz, 1H), 8.27 (dd, J=2.5, 1.5 Hz, 1H), 8.16 (d, J=2.7 Hz, 1H), 8.06 (dd, J=9.1, 1.2 Hz, 1H), 7.60 (dd, J=9.1, 4.6 Hz, 1H).

LCMS (ES): Found 174.2 [M+H]⁺.

NaH (60%, 49 mg, 1.21 mmol) was added to a solution of (3) (200 mg, 1.15 mmol) in DMF (8 mL) at 5° C. under N₂(g). The reaction mixture was stirred for 20 min then methyl 4-(bromomethyl)benzoate (344 mg, 1.5 mmol) in DMF (2 mL) was added. The stirring was continued at 70° C. under N₂(g) for 1 h. The reaction was cooled to rt, poured onto water (200 mL) and brine (50 mL), and the aqueous layer was extracted with EtOAc (2×150 mL). Combined organics were dried over Na₂SO₄, filtered and concentrated in vacuo. The residue was purified by flash column chromatography with heptane/EtOAc (1:0-0:1) then EtOAc/MeOH (1:0-4:1) yielded (4) (119 mg, 32%) as a brown oil.

¹H NMR (250 MHz, Chloroform-d), δ_{H} ppm: 8.85 (dd, J=4.6, 1.4 Hz, 1H), 8.56 (d, J=1.4 Hz, 1H), 8.25 (dd, J=2.6, 1.5 Hz, 1H), 8.17 (d, J=2.6 Hz, 1H), 7.89-7.97 (m, 2H), 7.48 (dd, J=9.1, 1.4 Hz, 1H), 7.42 (d, J=8.5 Hz, 2H), 7.33 (dd, J=9.1, 4.6 Hz, 1H), 5.64 (s, 2H), 3.86 (s, 3H).

LCMS (ES): Found 321.0 [M+H]⁺.

A solution of (4) (119 mg, 0.37 mmol) in 0.85M hydroxylamine in MeOH (10 mL) was stirred at rt for 72 h. After this time the solvent was concentrated to dryness and the residue purified by reverse phase HPLC to give Example MM (24 mg, 20%) as a beige solid.

¹H NMR (500 MHz, Methanol-d₄), δ_{H} ppm: 8.81 (dd, J=4.6, 1.2 Hz, 1H), 8.65 (d, J=1.4 Hz, 1H), 8.33 (dd, J=2.6, 1.5 Hz, 1H), 8.16 (d, J=2.6 Hz, 1H), 7.68 (m, 3H), 7.56 (dd, J=9.1, 4.6 Hz, 1H), 7.35 (d, J=8.2 Hz, 2H), 5.57 (s, 2H).

LCMS (ES): Found 322.2 [M+H]⁺.

### Example NN

### 3-Fluoro-N-hydroxy-4-{[(pyrazin-2-yl)(pyridazin-3-yl)amino]methyl}benzamide

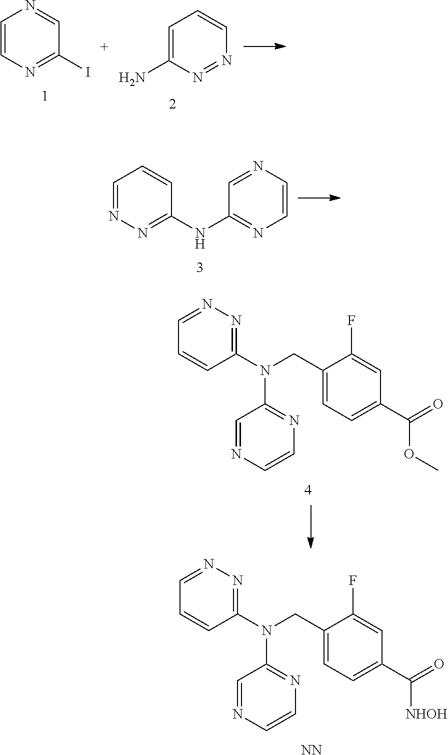

NaH (60%, 73 mg, 1.82 mmol) was added to a solution of (3) (300 mg, 1.73 mmol) in DMF (11 mL) at 5° C. under N₂(g). The reaction mixture was stirred for 20 min then methyl 4-(bromomethyl)-3-fluorobenzoate (556 mg, 2.25 mmol) in DMF (4 mL) was added. The stirring was continued at 70° C. under N₂(g) for 1 h. The reaction was cooled to rt and poured onto water (150 mL) and brine (25 mL), and the aqueous layer was extracted with EtOAc (150+100 mL). Combined organics were dried over Na₂SO₄, filtered and concentrated. The residue was purified by flash column chromatography with CH₂Cl₂/EtOAc (1:0-0:1) then EtOAc/MeOH (1:0-4:1) to yield (4) (141 mg, 24%) as a brown oil.

¹H NMR (500 MHz, Chloroform-d), δ_{H} ppm: 8.85 (dd, J=4.6, 1.3 Hz, 1H), 8.59 (d, J=1.4 Hz, 1H), 8.23 (dd, J=2.6, 1.5 Hz, 1H), 8.18 (d, J=2.6 Hz, 1H), 7.61-7.71 (m, 2H), 7.50 (dd, J=9.1, 1.3 Hz, 1H), 7.32-7.42 (m, 2H), 5.64 (s, 2H), 3.86 (s, 3H).

LCMS (ES): Found 339.9 [M+H]⁺.

A solution of (4) (141 mg, 0.42 mmol) in 0.85M hydroxylamine in MeOH (10 mL) was stirred at rt for 18 h. The solvent was concentrated to dryness and the residue purified by reverse phase HPLC to give Example NN (51 mg, 36%) as a beige solid.

¹H NMR (500 MHz, Methanol-d₄), δ_{H} ppm: 8.83 (dd, J=4.6, 1.1 Hz, 1H), 8.67 (d, J=1.3 Hz, 1H), 8.34 (dd, J=2.5, 1.5 Hz, 1H), 8.18 (d, J=2.6 Hz, 1H), 7.70 (dd, J=9.1, 1.2 Hz, 1H), 7.59 (dd, J=9.1, 4.6 Hz, 1H), 7.47 (d, J=11.7 Hz, 2H), 7.32 (t, J=8.0 Hz, 1H), 5.60 (s, 2H).

LCMS (ES): Found 341.0 [M+H]⁺.

### Example 00

### N-Hydroxy-4-([(3-methyl-1,2,4-thiadiazol-5-yl)(pyrazin-2-yl)amino]methyl)benzamide

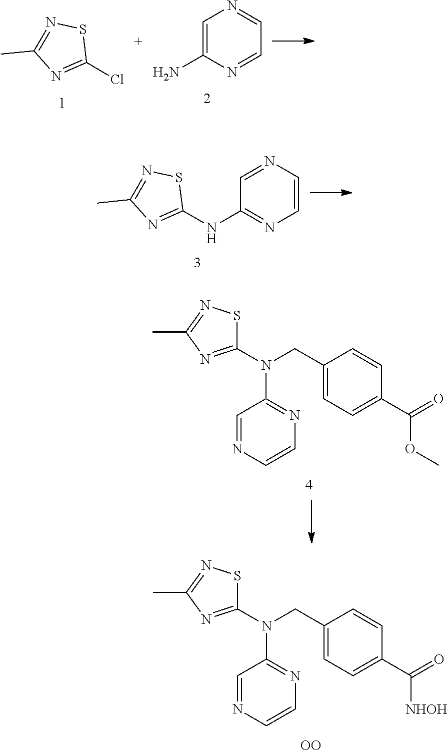

NaH (60%, 120 mg, 3.3 mmol) was added to a solution of (2) (140 mg, 1.47 mmol) in THF (10 mL) under N₂(g). The reaction mixture was stirred for 10 min then 5-chloro-3-methyl-1,2,4-thiadiazole (1) (190 mg, 1.41 mmol) was added. The mixture was heated up at 50° C. under N₂(g) for 24 h.

LCMS (ES): Found 194.0 [M+H]⁺.

To this mixture was added MCN (10 mL), methyl 4-(bromomethyl)benzoate (400 mg, 1.74 mmol) and potassium carbonate (350 mg, 1.65 mmol). Heating was then continued at 50° C. for 2 h. Once cooled, the mixture was partitioned between H₂O (10 mL) and EtOAc (3×20 mL). Combined organics were dried over Na₂SO₄, filtered and concentrated in vacuo. The residue was purified by flash column chromatography with Petrol/EtOAc (1:0-1:1) to yield (4) (300 mg, 60% over 2 steps) as a white solid.

¹H NMR (400 MHz, DMSO-d₆), δ_{H} ppm: 8.55-8.77 (m, 2H), 8.41 (s, 1H), 7.92 (d, J=7.9 Hz, 2H), 7.39 (d, J=7.9 Hz, 2H), 5.92 (s, 2H), 3.82 (s, 3H), 2.42 (s, 3H).

LCMS (ES): Found 342.0 [M+H]⁺.

A solution of (4) (174 mg, 0.51 mmol) in 0.85M hydroxylamine in MeOH (10 mL) was stirred at 70° C. for 8 h. The solvent was concentrated to dryness and the residue purified by reverse phase HPLC to give Example OO (44 mg, 25%).

¹H NMR (400 MHz, DMSO-d₆), δ_{H} ppm: 11.45-10.94 (m, 1H), 9.43-8.80 (m, 1H), 8.70 (d, J=1.3 Hz, 1H), 8.61 (dd, J=2.6, 1.5 Hz, 1H), 8.40 (d, J=2.6 Hz, 1H), 7.70 (d, J=8.5 Hz, 2H), 7.31 (d, J=8.3 Hz, 2H), 5.88 (s, 2H), 2.43 (s, 3H).

LCMS (ES): Found 343.0 [M+H]⁺.

### Example PP

### N-Hydroxy-4-{[(4-methoxypyridin-2-yl)pyrazin-2-yl)amino]methyl}benzamide

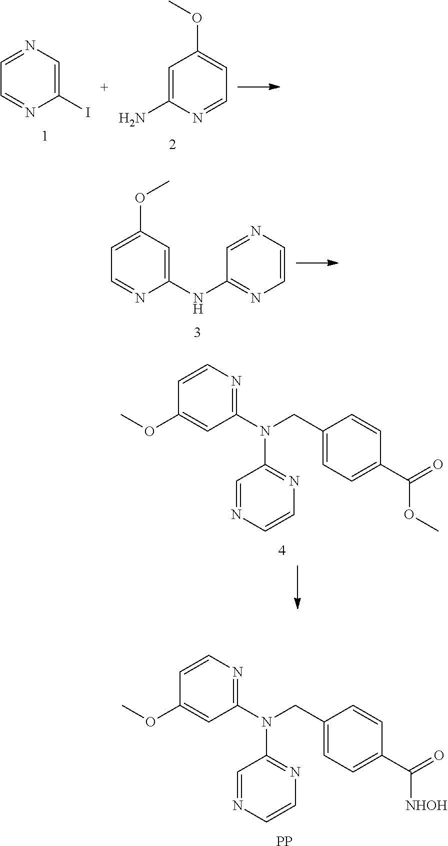

A solution of 2-iodopyrazine (1) (1.34 g, 6.51 mmol), 4-methoxypyridin-2-amine (2) (0.85 g, 6.83 mmol), Cs₂CO₃ (4.24 g, 13.01 mmol) and Xantphos (0.17 g, 0.29 mmol) in dioxane (22 mL) was purged with N₂(g) for 10 min then Pd₂(dba)₃ (0.12 g, 0.13 mmol) was added, re-purged for ˜5 min and reaction was heated to 90° C. for 4 h. Once cooled down to rt, the mixture was partitioned between H₂O (150 mL) and EtOAc (3×120 mL). Combined organics were dried over Na₂SO₄, filtered and concentrated in vacuo. The residue was purified by flash column chromatography with CH₂Cl₂/EtOAc (9:1-0:1) to yield (3) (809 mg, 61%) as a yellow solid.

¹H NMR (500 MHz, Chloroform-d), δ_{H} ppm: 8.70 (d, J=1.3 Hz, 1H), 8.11-8.22 (m, 3H), 8.08 (d, J=2.7 Hz, 1H), 7.43 (d, J=2.2 Hz, 1H), 6.52 (dd, J=5.8, 2.3 Hz, 1H), 3.88 (s, 3H).

LCMS (ES): Found 203.2 [M+H]⁺.

NaH (60%, 42 mg, 1.04 mmol) was added to a solution of (3) (200 mg, 0.99 mmol) in DMF (7 mL) at rt under N₂(g). The reaction mixture was stirred for 30 min then methyl 4-(bromomethyl)-3-fluorobenzoate (249 mg, 1.09 mmol) in DMF (2 mL) was added. The reaction was heated up to 70° C. under N₂(g) for 2 h, then at rt overnight. The reaction was cooled to rt and partitioned between H₂O (150 mL) and EtOAc (2×100 mL). Combined organics were dried over Na₂SO₄, filtered and concentrated in vacuo. The residue was purified by flash column chromatography with CH₂Cl₂/EtOAc (1:0-0:1) to yield (4) (173 mg, 50%) as a viscous oil.

¹H NMR (300 MHz, Chloroform-d), δ_{H} ppm: 8.63 (dd, J=1.4 Hz, 1H), 8.14-8.22 (m, 2H), 8.01 (d, J=2.6 Hz, 1H), 7.92 (d, J=8.2 Hz, 2H), 7.39 (d, J=8.2 Hz, 2H), 6.61 (d, J=2.1 Hz, 1H), 6.54 (dd, J=5.8, 2.2 Hz, 1H), 5.46 (s, 2H), 3.85 (s, 3H), 3.75 (s, 3H).

LCMS (ES): Found 350.9 [M+H]⁺.

A solution of (4) (173 mg, 0.49 mmol) in 0.85M hydroxylamine in MeOH (10 mL) was stirred at rt for 72 h. The solvent was concentrated to dryness and the residue purified by reverse phase HPLC to give Example PP (15 mg, 9%).

¹H NMR (500 MHz, Methanol-d₄), δ_{H} ppm: 8.46 (d, J=1.4 Hz, 1H), 8.24 (dd, J=2.6, 1.5 Hz, 1H), 8.14 (d, J=5.9 Hz, 1H), 8.00 (d, J=2.7 Hz, 1H), 7.65 (d, J=8.3 Hz, 2H), 7.42 (d, J=8.3 Hz, 2H), 6.79 (d, J=2.2 Hz, 1H), 6.73 (dd, J=5.9, 2.2 Hz, 1H), 5.45 (s, 2H), 3.82 (s, 3H).

LCMS (ES): Found 352.0 [M+H]⁺.

### Example QQ

### N-Hydroxy-4-{[(pyrazin-2-yl)[6-(trifluoromethyl)pyrazin-2-yl]amino]methyl}benzamide

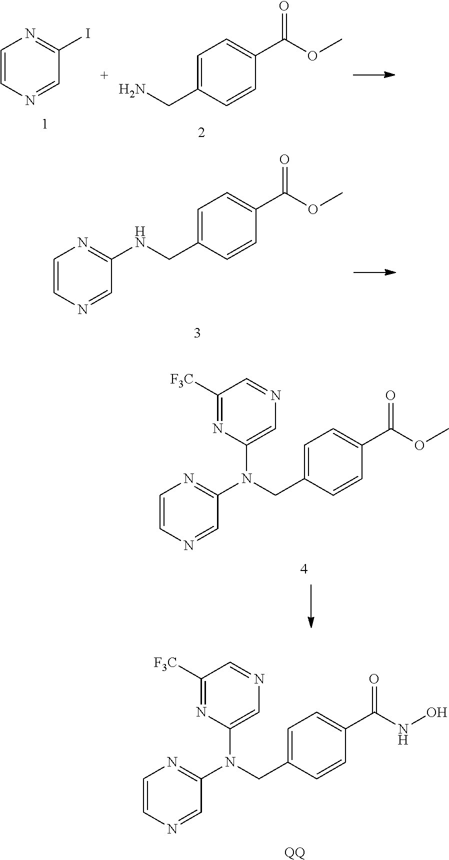

To a solution of methyl 4-(aminomethyl)benzoate hydrochloride (1.47 g, 7.3 mmol) in DMSO (14 mL) was added 2-iodopyrazine (1 g, 4.9 mmol) followed by K₂CO₃ (1.7 g, 12.1 mmol) under Ar(g). After 2 min vigorous stirring, CuI (46 mg, 0.2 mmol) was added and the mixture was left to stir at rt overnight. It was partitioned between EtOAc (150 mL) and 50% brine (50 mL) and the organic layer separated, the aqueous layer was extracted with EtOAc (2×15 mL), before the combined organic phase was washed with 50% brine (15 mL), dried (MgSO₄) and concentrated in vacuo. The residue was purified by flash column chromatography with Hexane/EtOAc (7:3-0:1) to yield (3) (670 mg, 57%) as a white solid.

¹H NMR (300 MHz, CHLOROFORM-d) δ_{H} ppm: 7.76-8.11 (m, 5H), 7.43 (d, J=8.5 Hz, 2H), 5.01-5.16 (m, 1H), 4.66 (d, J=5.8 Hz, 2H), 3.92 (s, 3H).

LCMS (ES): Found 352.0 [M+H]⁺.

To compound (2) (60 mg, 0.25 mmol), Pd₂(dba)₃ (11 mg, 0.01 mmol), (±)-BINAP (15 mg, 0.025 mmol) and Cs₂CO₃ (241 mg, 0.74 mmol) was added a solution of 2-chloro-6-(trifluoromethyl)pyrazine (90 mg, 0.49 mmol) in dioxane (2 mL) under Ar(g). The reaction mixture was heated at 90° C. for 4 h then allowed to cool to rt overnight EtOAc (15 mL), water (4 mL) and brine (2 mL) were then added. The organic phase was separated extracting the aqueous layer with EtOAc (10 mL). The combined organic phases were dried (MgSO₄) and concentrated in vacuo to give a crude residue (153 mg). The residue was scavenged by dissolving in CH₂Cl₂/MeOH (1:1, 10 mL) followed by the addition of MP-TMT (370 mg, 0.68 mmol/g). The mixture was agitated for 24 h before filtering off the resin, washing with CH₂Cl₂/MeOH (1:1, 2×5 mL). The filtrate was then concentrated in vacuo to give crude (3) (132 mg), as a brown solid which was used directly in the next step.

To a solution of crude (3) (132 mg total, containing maximum 0.25 mmol) in THF/MeOH (1:1, 4 mL) was added NH₂OH solution (50% wt. H₂O, 0.306 mL, 5 mmol) followed by NaOH (6M, 0.083 mL, 0.5 mmol). After 50 min stirring at rt, KHSO₄ (M, 2 mL), water (5 mL) and CH₂Cl₂ (6 mL) were added. The organic phase was separated and the aqueous layer was extracted with CH₂Cl₂ (2×5 mL). The combined organic phase was dried (MgSO₄) and concentrated in vacuo to give a yellow solid. Purification by reverse phase C-18 chromatography with MeCN/H₂O (19:1-1:1) gave Example QQ (81 mg, 83% over 2 steps) as a light brown solid.

¹H NMR (DMSO-d₆) δ_{H} ppm: 8.93 (s, 1H), 8.88 (d, J=1.7 Hz, 1H), 8.62 (s, 1H), 8.42 (dd, J=2.6, 1.5 Hz, 1H), 8.34 (d, J=2.6 Hz, 1H), 7.62 (d, J=8.3 Hz, 2H), 7.27 (d, J=8.3 Hz, 2H), 5.46 (s, 2H).

LCMS (ES): Found 391.1 [M+H]⁺.

### Example RR

### 4-(([5-(6-Aminopyridin-3-yl)pyridin-2-yl](pyrazin-2-yl)amino)methyl)-N-hydroxybenzamide

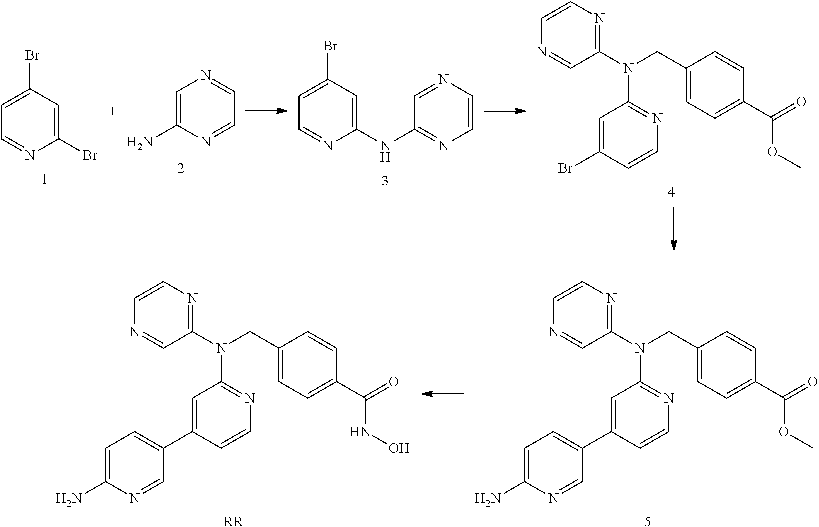

A mixture of 2,4-dibromopyridine (1) (5.0 g, 21.1 mmol), pyrazin-2-amine (2) (2.21 g, 23.22 mmol), Cs₂CO₃ (15.1 g, 46.4 mmol) and Xantphos (611 mg, 1.05 mmol) was suspended in dioxane (50 mL). The mixture was flushed with N₂(g) for 1 min before Pd₂(dba)₃ (386 mg, 0.422 mmol) was added. Mixture was flushed again with N₂(g) and it was heated up to 90° C. overnight. Once cooled, the mixture was partitioned between H₂O (150 mL) and EtOAc (3×150 mL). The combined organic extracts were washed with brine, dried with MgSO₄, filtered and concentrated in vacuo. Purification by flash column chromatography with heptane/EtOAc (9:1-2:3) to yield (3) (2.6 g, 49%) as pale yellow solid.

¹H NMR (500 MHz, Chloroform-d), δ_{H} ppm: 8.74 (d, J=1.3 Hz, 1H), 8.22 (dd, J=2.6, 1.5 Hz, 1H), 8.15 (d, J=2.7 Hz, 1H), 8.11 (d, J=5.4 Hz, 1H), 8.07 (d, J=1.5 Hz, 1H), 7.63 (s, 1H), 7.10 (dd, J=5.4, 1.6 Hz, 1H).

LCMS (ES): Found 251.0-253.0 [M+H]⁺.

To a solution of (3) (1.08 g, 4.3 mmol) in DMF (15 mL) cooled to 0° C. under N₂(g) was added NaH (60%, 206 mg, 5.16 mmol). The mixture was stirred for 30 min. Then, a solution of methyl 4-(bromomethyl)benzoate (1.08 g, 4.73 mmol) in DMF (5 mL) was added and the mixture was heated up to 50° C. for 1.5 h. Once cooled down, the reaction was partitioned between H₂O (150 mL) and EtOAc (3×150 mL). The combined organic extracts were washed with brine, dried with MgSO₄, filtered and concentrated in vacuo. Purification by flash column chromatography with heptane/EtOAc (9:1-2:3) to yield (4) (915 mg, 53%) as a white solid.

¹H NMR (500 MHz, Chloroform-d), δ_{H} ppm: 8.66 (d, J=1.4 Hz, 1H), 8.25 (dd, J=2.5, 1.6 Hz, 1H), 8.15 (d, J=5.3 Hz, 1H), 8.13 (d, J=2.6 Hz, 1H), 7.95 (d, J=8.3 Hz, 2H), 7.39 (d, J=8.3 Hz, 2H), 7.33 (d, J=1.4 Hz, 1H), 7.10 (dd, J=5.3, 1.5 Hz, 1H), 5.49 (s, 2H), 3.88 (s, 3H).

LCMS (ES): Found 399.0-401.0 [M+H]⁺.

To a suspension of (4) (200 mg, 0.50 mmol), 5-(4,4,5,5-tetramethyl-1,3,2-dioxaborolan-2-yl)pyridin-2-amine (132.3 mg, 0.6 mmol) and Cs₂CO₃ (326 mg, 1.0 mmol) in DMF (4 mL) and H₂O (1 mL) was added Pd(PPh₃)₄ (58 mg, 0.05 mmol). The mixture was flushed with N₂(g) then it was heated up to 90° C. for 2 h. Once cooled down, H₂O (20 mL) was added and a precipitate was left to settle at rt for 72 h. After filtration, washings with H₂O (2 mL) and drying, (5) was obtained as a brown solid (219 mg, quant.).

¹H NMR (500 MHz, Methanol-d₄), δ_{H} ppm: 8.54 (s, 1H), 8.31 (d, J=5.3 Hz, 1H), 8.25-8.28 (m, 1H), 8.23 (d, J=2.3 Hz, 1H), 8.02 (d, J=2.6 Hz, 1H), 7.92 (d, J=8.2 Hz, 2H), 7.77 (dd, J=8.8, 2.4 Hz, 1H), 7.50 (s, 1H), 7.48 (d, J=5.5 Hz, 2H), 7.32 (d, J=5.4 Hz, 1H), 6.65 (d, J=8.8 Hz, 1H), 5.55 (s, 2H), 3.86 (s, 3H).

LCMS (ES): Found 413.0 [M+H]⁺.

A solution of (5) (219 mg, 0.53 mmol) in 0.85M NH₂OH in MeOH (5 mL) was stirred at rt overnight. The volatiles were then removed in vacuo and the residue was purified by reverse prep HPLC to give Example RR (19 mg, 8%) as pale yellow solid.

1H NMR (500 MHz, DMSO-d₆), δ_{H} ppm: 8.63 (d, J=1.4 Hz, 1H), 8.35 (d, J=2.3 Hz, 1H), 8.27-8.28 (m, 1H), 8.26-8.27 (m, 1H), 8.07 (d, J=2.6 Hz, 1H), 7.76 (d, J=2.6 Hz, 1H), 7.61 (d, J=8.3 Hz, 2H), 7.51 (s, 1H), 7.30 (dd, J=5.3, 1.5 Hz, 1H), 7.26 (d, J=8.2 Hz, 2H), 6.52 (d, J=8.7 Hz, 1H), 6.36 (s, 2H), 5.45 (s, 2H).

LCMS (ES): Found 414.0 [M+H]⁺.

### Example SS

### 4-(([5-(2-Aminopyridin-4-yl)pyridin-2-yl](pyrazin-2-yl)amino)methyl)-N-hydroxybenzamide

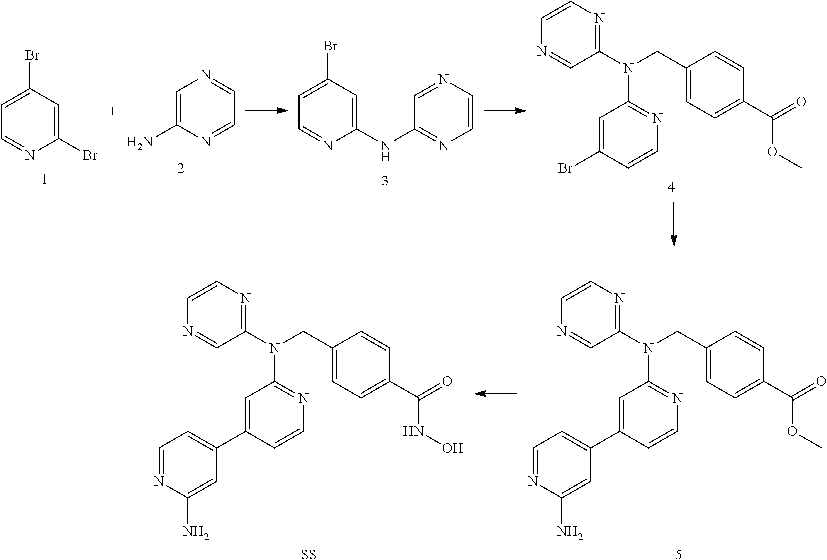

To a suspension of (4) (200 mg, 0.50 mmol), 4-(4,4,5,5-tetramethyl-1,3,2-dioxaborolan-2-yl)pyridin-2-amine (132.3 mg, 0.6 mmol) and Cs₂CO₃ (326 mg, 1.0 mmol) in DMF (4 mL) and H₂O (1 mL) was added Pd(PPh₃)₄ (58 mg, 0.05 mmol). The mixture was flushed with N₂(g) then it was heated up to 90° C. for 2 h. Once cooled down, H₂O (20 mL) was added and a precipitate was left to settle at rt for 3 h. After filtration, washings with H₂O (2 mL) and drying, a pale orange solid was obtained, which was purified by flash column chromatography with heptane/EtOAc (4:1-0:1) then EtOAc/MeOH (1:0-7:3) to give (5) (82 mg, 40%) as a yellow solid.

1H NMR (500 MHz, Methanol-d₄), δ_{H} ppm: 8.60 (s, 1H), 8.41 (d, J=5.2 Hz, 1H), 8.29 (d, J=1.3 Hz, 1H), 8.06 (d, J=2.5 Hz, 1H), 7.97 (d, J=5.4 Hz, 1H), 7.93 (d, J=8.3 Hz, 2H), 7.53 (s, 1H), 7.49 (d, J=8.1 Hz, 2H), 7.34 (d, J=5.2 Hz, 1H), 6.81-6.84 (m, 1H), 6.81 (s, 1H), 5.58 (s, 2H), 3.86 (s, 3H).

LCMS (ES): Found 413.0 [M+H]⁺.

A solution of (5) (82 mg, 0.20 mmol) in 0.85M NH₂OH in MeOH (5 mL) was stirred at rt overnight. The volatiles were then removed in vacuo and the residue was purified by reverse prep HPLC to give Example SS (19 mg, 8%) as white solid.

1H NMR (500 MHz, Methanol-d₄), δ_{H} ppm: 8.59 (d, J=1.4 Hz, 1H), 8.39 (d, J=5.2 Hz, 1H), 8.29 (dd, J=2.7, 1.5 Hz, 1H), 8.05 (d, J=2.7 Hz, 1H), 7.97 (d, J=5.5 Hz, 1H), 7.66 (d, J=8.3 Hz, 2H), 7.49 (s, 1H), 7.45 (d, J=8.2 Hz, 2H), 7.32 (dd, J=5.2, 1.2 Hz, 1H), 6.82 (dd, J=5.5, 1.3 Hz, 1H), 6.78 (s, 1H), 5.55 (s, 2H).

LCMS (ES): Found 414.0 [M+H]⁺.

### Example TT

### N-hydroxy-4-({[2′-(methylamino)-[4,4′-bipyridine]-2-yl](pyrazin-2-yl)amino}methyl)benzamide

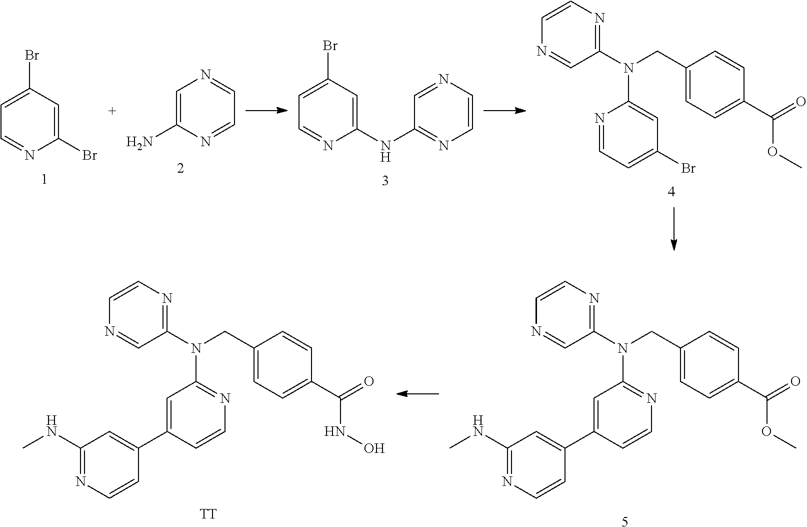

To a suspension of (4) (120 mg, 0.3 mmol), N-methyl-4-(4,4,5,5-tetramethyl-1,3-dioxolan-2-yl)pyridin-2-amine (84 mg, 0.36 mmol) and Cs₂CO₃ (196 mg, 0.6 mmol) in DMF (2 mL) and H₂O (0.5 mL) was added Pd(PPh₃)₄ (58 mg, 0.05 mmol). The mixture was flushed with N₂(g) then it was heated up to 90° C. for 4 h. Once cooled down, H₂O (10 mL) was added and the reaction was stirred for 20 min.

After filtration, washings with MCN (2 mL) and drying, a black solid was obtained, which was purified by preparative HPLC to give (5) (80 mg, 59%) as a white solid.

1H NMR (500 MHz, DMSO-d₆), δ_{H} ppm: 8.70 (d, J=1.4 Hz, 1H), 8.39 (d, J=5.2 Hz, 1H), 8.29 (dd, J=2.6, 1.5 Hz, 1H), 8.14 (d, J=2.6 Hz, 1H), 8.07 (d, J=5.3 Hz, 1H), 7.87 (d, J=8.4 Hz, 2H), 7.54-7.56 (m, 1H), 7.50 (d, J=8.3 Hz, 2H), 7.32 (dd, J=5.2, 1.4 Hz, 1H), 6.77 (dd, J=5.3, 1.5 Hz, 1H), 6.65-6.67 (m, 1H), 6.61 (d, J=5.2 Hz, 1H), 5.56 (s, 2H), 3.80 (s, 3H), 2.80 (d, J=4.9 Hz, 3H).

LCMS (ES): Found 427.5 [M+H]⁺.

To a solution of (5) (80 mg, 0.20 mmol) in MeOH/THF (1:1, 2 mL) was added hydroxylamine (50% w/w in H₂O; 0.11 mL, 3.75 mmol) followed by 6N NaOH (0.063 mL, 0.38 mmol). The mixture was stirred at rt for 3 h. Then, 1M KHSO₄ (2 mL) was added followed by H₂O (6 mL). It was extracted with IPA/Chloroform (1:2, 3×20 mL). The combined organic extracts were washed with brine, dried with MgSO₄, filtered and concentrated in vacuo. Purification by preparative HPLC yielded Example TT (21 mg, 25%) as a pale orange solid.

1H NMR (500 MHz, Methanol-d₄), δ_{H} ppm: 11.08 (br. s., 1H), 8.69 (dd, J=6.3, 1.4 Hz, 1H), 8.39 (dd, J=5.0, 1.4 Hz), 8.28-8.32 (m, 1H), 8.13 (dd, J=6.0, 2.6 Hz, 1H), 8.07 (dd, J=5.2, 3.3 Hz, 1H), 7.63-7.67 (m, 1H), 7.58 (d, J=8.4 Hz, 1H), 7.53 (m, 1H), 7.42 (d, J=8.4 Hz, 1H), 7.36 (d, J=8.4 Hz, 1H), 7.31 (ddd, J=8.5, 5.3, 1.4, 1H), 6.65 (ddd, J=8.5, 5.4, 1.5 Hz), 6.66 (d, J=9.1 Hz, 1H), 6.58-6.63 (m, 1H), 5.51 (m, 1H), 2.80 (m, 3H).

LCMS (ES): Found 428.2 [M+H]⁺.

### Example UU

### 4-[({[4,4′-bipyridine]-2-yl}(pyrazin-2-yl)amino)methyl]-N-hydroxybenzamide

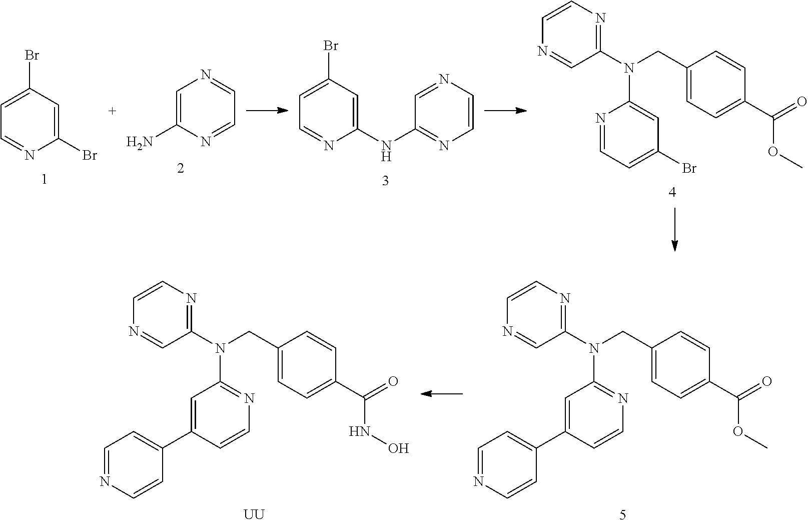

To a suspension of (4) (120 mg, 0.3 mmol), (pyridin-4-yl)boronic acid (49 mg, 0.36 mmol) and Cs₂CO₃ (196 mg, 0.6 mmol) in DMF (2 mL) and H₂O (0.5 mL) was added Pd(PPh₃)₄ (35 mg, 0.03 mmol). The mixture was flushed with N₂(g) then it was heated up to 90° C. for 4 h. Once cooled down, H₂O (10 mL) was added and the reaction was stirred for 20 min. After filtration, a gum was obtained, which was purified by preparative HPLC then SCX column to give (5) (82 mg, 65%) as a colourless oil.

LCMS (ES): Found 398.5 [M+H]⁺.

To a solution of (5) (82 mg, 0.21 mmol) in MeOH/THF (1:1, 2 mL) was added hydroxylamine (50% w/w in H₂O; 0.15 mL, 0.42 mmol) followed by 6N NaOH (0.08 mL, 0.42 mmol). The mixture was stirred at rt for 2 h. The volatiles were then removed in vacuo and the residue was purified by reverse prep HPLC to give Example UU (39 mg, 48%) as white solid.

1H NMR (500 MHz, DMSO-d₆), δ_{H} ppm: 11.05 (br. s., 1H), 8.95 (br. s., 1H), 8.68-8.71 (m, 3H), 8.44 (d, J=5.2 Hz, 1H), 8.28-8.31 (m, 1H), 8.14 (d, J=2.6 Hz, 1H), 7.72-7.78 (m, 3H), 7.64 (d, J=8.2 Hz, 2H), 7.47 (dd, J=5.2, 1.4 Hz, 1H), 7.42 (d, J=8.0 Hz, 2H), 5.55 (s, 2H).

LCMS (ES): Found 399.4 [M+H]⁺.

Biochemical Assay and Data

1) Assay

i. Biochemical Assay Description

Activity against all zinc-dependent HDACs 1 to 11 was assessed by using an acetylated AMC-labeled peptide substrate. The substrate RHKK(Ac)AMC was used for HDAC1, 2, 3, 6, 10 and 11; for HDAC8, the substrate used was RHKAcKAc. Activity against HDAC4, 5, 7, 9 was determined using a class IIa-specific substrate, Acetyl-Lys(trifluoroacetyl)-AMC (Lahm et al, 2007, PNAS, 104, 17335-17340). All assays were based on the AMC-labeled substrate and developer combination.

The protocol involved a two-step reaction: first, the substrate with the acetylated lysine side chain is incubated with a sample containing HDAC activity, to produce the deacetylated products, which are then digested in the second step by the addition of developer to produce the fluorescent signal proportional to the amount of deacetylated substrates.

ii. Enzymes

Human HDAC1 (GenBank Accession No. NM_004964), full length with C-terminal His-tag and C-terminal FLAG-tag, MW=56 kDa, expressed in baculovirus expression system.

Human HDAC2 (GenBank Accession No. NM_001527), full length with C-terminal His-tag, MW=56 kDa or full length with C-terminal GST-tag, MW=82.9 kDa expressed inbaculovirus expression system.

Complex of human HDAC3 (GenBank Accession No. NM_003883), full length with C-terminal His tag, MW=49.7 kDa, and human NCOR2 (amino acid 395-489) (GenBank Accession No. NM_006312), N-terminal GST tag, MW=37.6 kDa, co-expressed in baculovirus expression system.

Human HDAC4 (GenBank Accession No. NM_006037), amino acids 627-1085 with N-terminal GST tag, MW=75.2 kDa, expressed in baculovirus expression system.

Human HDAC5 (GenBank Accession No. NM_005474), full length with N-terminal GST tag, MW=150 kDa, or full length with C-terminal His tag, MW=51.1 kDa, expressed in baculovirus expression system.

Recombinant human HDAC6 (GenBank Accession No. BC069243), full length, MW=159 kDa, was expressed by baculovirus in Sf9 insect cells using an N-terminal GST tag.

Human HDAC7 (GenBank Accession No. AY302468), (amino acids 518-end) with N-terminal GST tag, MW=78 kDa, expressed in baculovirus expression system.

Human HDAC8 (GenBank Accession No. NM_018486), full length with C-terminal His tag, MW=42.6 kDa, expressed in a baculovirus expression system.

Human HDAC9 (GenBank Accession No. NM_178423), amino acids 604-1066 with C-terminal His tag, MW=50.7 kDa, expressed in baculovirus expression system.

Human HDAC10 (a.a. 1-481), GenBank Accession No. NM_032019 with N-terminal GST tag and C-terminal His tag, MW=78 kDa, expressed in baculovirus expression system.

Human HDAC11 (full length) (GenBank Accession No. NM_024827) with N-terminal GST tag, MW=66 kDa, expressed in baculovirus expression system.

iii. Reaction Conditions

Either, Reaction Conditions A:

Assay Buffer 50 mM Tris-HCl, pH8.0, 137 mM NaCl, 2.7 mM KCl, 1 mM MgCl₂. Before use, 1 mg/mL BSA and DMSO are added.

HDAC1: 2.68 nM HDAC1 and 50 μM HDAC substrate are in the reaction buffer with 1% DMSO final. Incubate for 2 hours at 30° C.

HDAC2: 3.33 nM HDAC2 and 50 μM HDAC substrate are in the reaction buffer with 1% DMSO final. Incubate for 2 hours at 30° C.

HDAC3: 1.13 nM HDAC3 and 50 μM HDAC substrate are in the reaction buffer with 1% DMSO final. Incubate for 2 hours at 30° C.

HDAC6: 0.56 nM HDAC6 and 50 μM HDAC substrate are in the reaction buffer with 1% DMSO final. Incubate for 2 hours at 30° C.

HDAC8: 46.4 nM HDAC8 and 50 μM HDAC8 substrate are in the reaction buffer with 1% DMSO final. Incubate for 2 hours at 30° C.

HDAC10: 96.15 nM HDAC10 and 50 μM HDAC substrate are in the reaction buffer with 1% DMSO final. Incubate for 2 hours at 30° C.

HDAC11: 227.27 nM HDAC11 and 50 μM HDAC substrate are in the reaction buffer with 1% DMSO final. Incubate for 2 hours at 30° C.

For class IIa HDACs, assay buffer is the same.

Other reaction conditions are as follows:

HDAC4: 0.03 nM HDAC4 and 50 mM Class IIa HDAC substrate are in the reaction buffer with 1% DMSO final. Incubate for 30 minutes at room temperature.

HDAC5: 0.67 nM HDAC5 and 50 mM Class IIa HDAC substrate are in the reaction buffer with 1% DMSO final. Incubate for 30 minutes at room temperature.

HDAC7: 0.26 nM HDAC7 and 50 mM Class IIa HDAC substrate are in the reaction buffer with 1% DMSO final. Incubate for 30 minutes at room temperature.

HDAC9: 2.37 nM HDAC9 and 50 mM Class IIa HDAC substrate are in the reaction buffer with 1% DMSO final. Incubate for 30 minutes at room temperature.

Or, Reaction Conditions B:

Assay Buffer: 50 mM Tris-HCl, pH8.0, 137 mM NaCl, 2.7 mM KCl, 1 mM MgCl₂. Before use, 1 mg/mL BSA and DMSO are added.

HDAC1: 0.3 ng/ul HDAC1 and 50 μM HDAC substrate are in the reaction buffer with 1% DMSO final. Incubate for 1 hour at 30° C.

HDAC2: 0.07 ng/ul HDAC2 and 50 μM HDAC substrate are in the reaction buffer with 1% DMSO final. Incubate for 1 hour at 30° C.

HDAC3: 0.1 ng/ul HDAC3 and 50 μM HDAC substrate are in the reaction buffer with 1% DMSO final. Incubate for 1 hour at 30° C.

HDAC6: 0.3 ng/ul HDAC6 and 50 μM HDAC substrate are in the reaction buffer with 1% DMSO final. Incubate for 1 hours at 30° C.

HDAC8: 1 ng/ul HDAC8 and 100 μM HDAC8 substrate are in the reaction buffer with 1% DMSO final. Incubate for 2 hours at 30° C.

HDAC10: 12 ng/ul HDAC10 and 50 μM HDAC substrate are in the reaction buffer with 1% DMSO final. Incubate for 2 hours at 30° C.

HDAC11: 5 ng/ul HDAC11 and 50 μM HDAC substrate are in the reaction buffer with 1% DMSO final. Incubate for 30 minutes at 30° C.

For class IIa HDACs, assay buffer is the same.

Other reaction conditions are as follows:

HDAC4: 0.004 ng/ul HDAC4 and 50 μM Class IIa HDAC substrate are in the reaction buffer with 1% DMSO final. Incubate for 30 minutes at room temperature.

HDAC5: 0.05 ng/ul HDAC5 and 50 μM Class IIa HDAC substrate are in the reaction buffer with 1% DMSO final. Incubate for 30 minutes at room temperature.

HDAC7: 0.001 ng/ul HDAC7 and 50 μM Class IIa HDAC substrate are in the reaction buffer with 1% DMSO final. Incubate for 30 minutes at room temperature.

HDAC9: 0.06 ng/ul HDAC9 and 50 μM Class IIa HDAC substrate are in the reaction buffer with 1% DMSO final. Incubate for 30 minutes at room temperature.

Control Inhibitor Trichostatin A (TSA)

Fluorescent Deacetylated Standard: Biomol, Cat # KI-142;

For Standard Control, compound is added at assay concentration to 2.5 uM Fluorescent Deacetylated Standard; 10 doses in 6 uL

For Fluorescence Background Control, compound is added at assay concentrations to 50 mM HDAC substrate; 10 doses in 6 uL.

Fluorescence background signal is then subtracted from compound data signal. % Conversion must be between 5% and 15% to obtain optimum result.

iv. Assay Procedure

Stage 1: Deacetylation of substrate by incubation of HDAC enzymes with compounds

Stage 2: Development by addition of Developer to digest the deacetylated substrate, and generate the fluorescent color; Detection: 360/460 Ex/Em

2) Inhibition of HDAC Enzymes

IC₅₀ (nM) HDAC

| Example | 1 | 6 |
|---|---|---|
| A | **** | * |
| B | **** | * |
| C | *** | * |
| D | *** | * |
| E | *** | * |
| F | **** | * |
| G | **** | * |
| H | **** | * |
| I | *** | * |
| J | **** | * |
| K | **** | * |
| L | **** | * |
| M | **** | * |
| N | **** | * |
| O | **** | * |
| P | **** | * |
| Q | *** | * |
| R | **** | * |
| S | **** | *** |
| T | **** | *** |
| U | *** | * |
| V | **** | * |
| W | **** | * |
| X | **** | * |
| Y | **** | * |
| Z | **** | * |
| AA | *** | * |
| BB | *** | * |
| CC | **** | ** |
| DD | *** | * |
| EE | *** | * |
| FF | **** | * |
| GG | *** | * |
| HH | *** | * |
| II | *** | * |
| JJ | *** | * |
| KK | *** | * |
| LL | **** | * |
| MM | **** | * |
| NN | **** | * |
| OO | *** | * |
| PP | *** | * |
| RR | *** | * |
| SS | *** | * |
| TT | *** | * |
| UU | *** | * |

Key:
**** ≥10 uM
*** ≤10 uM ≥ 1 uM
** ≤1 uM ≥ 500 nM
* ≤500 nM

Combination Data

Introduction

Data for an in vitro combination study are provided below.

The effects on the growth of a panel of cancer cell lines of a HDAC inhibitor which is Example GG (hereinafter referred to as Compound A) as disclosed herein alone or in combination with the following agents were tested:
i. Velcade (Bortezomib), a proteasome inhibitor (In MM1.R Multiple Myeloma (MM) cells (study LNB 013_070_210814 and 013_051_140814, Karus) and in KMS-12-BM, OPM-2, RPMI-8226, U266 and LP-1 MM cel lines (study 10922, ProQinase))
ii. Kyprolis (Carfilzomib), a proteasome inhibitor (In KMS-12-BM, OPM-2, RPMI-8226, U266 and LP-1 MM cel lines (study 10922, ProQinase))
iii. Revllmid (Lenalidomide), an immunomodulatory (IMiD) agent (In MM1.R multiple myeloma (MM) cells (study LNB 011_174_180914, Karus) and in KMS-12-BM, OPM-2, RPMI-8226, U266 and LP-1 MM cell lines (study 10922, ProQinase))
iv. Imnovid (Pomalidomide), an immunomodulatory (IMiD) agent (In KMS-12-BM, OPM-2, RPMI-8226, U266 and LP-1 MM cel lines (study 10922, ProQinase))
v. Opdivo (Nivolumab), an anti-PD-1 agent (study KRS018-01-b (DiscoverX)

Materials and Methods

Studies LNB 013_070_210814, 013_051_140814 and 011_174_180914 (Karus)

Proliferation Assay

MM.1R cells were maintained in RPMI1640 (Life Tech)+10% FCS+2 mM glutamine & penicillin (10 μg/mL) and streptomycin (100 mg/mL). 5000 cells per well in 100 pL (5×10⁴ cells mL⁻¹) were plated into 96 well tissue culture plates (Corning).

Compounds were diluted to 2× final assay concentration in media to a final DMSO concentration 0.26% (1.3% for Compound A-Revlimid combination). 24 h after cell seeding, 100 pL 2× compounds or DMSO control were added to cells (final DMSO concentration 0.26%, control untreated cells received 100 pL of media). Cells were exposed to compounds alone or in combination at a constant ratio and incubated at 37° C. in a humidified atmosphere containing 5% CO₂ for 96 h (for Compound A in combination with Revlimid, cells were exposed to Compound A for 24 h prior to addition of Revlimid and exposed to both agents for a further 72 h).

Measurement of the impact of compounds on cel viability was performed using CyQuant Assay (Life Tech). Briefly, assay plates were centrifuged at 1300 rpm for 3 mins and media removed from wells. Cells were washed once with PBS, re-centrifuged and PBS aspirated prior to freezing at −80° C. for a minimum of 1 h.

Plates were fully thawed at room temp prior to addition of CyQuant GR reagent-cell lysis buffer mix. Cells were lysed by incubation without exposure to light at room temperature for 3 mins. Fluorescence (480 nm excitation/520 nm emission filter set) was quantified using a Varioskan flash plate reader.

Data Analysis

Percentage of inhibition of cel viability was calculated against the mean of the DMSO treated controls and an IC₅₀ value for inhibition of cell growth calculated using GraphPad Prism software by nonlinear regression using 0% as bottom constraint and 100% as top constraint. Combination Index (CI) values as a measure of synergy were generated using Calcusyn software.

Study 10922 (ProQinase)

Proliferation Assay

KMS-12-BM, OPM-2, RPMI-8226, U266 and LP-1 cel lines were cultured in RPMI-1640 containing 10% FCS and penicillin/streptomycin. For proliferation assays, 5000 cells well⁻¹ were seeded in 150 μL medium in 96-well cell culture plate and incubated at 37° C. overnight prior to addition of compounds. Compounds or DMSO controls were diluted in medium at 16-fold (mono treatment) or 32-fold (combination) of the final assay concentration. 24 h after cel seeding, 10 pL (mono treatment) or 5 pL (combination) of each diluted compound, DMSO (final assay concentration 0.1%) or 10 μM staurosporine controls were added to the cells (1:16 or 1:32 dilution) and incubated for 72 h (or 96 h for KA507 in combination with Imnovid and Revlimid) at 37° C. and 5% CO₂.

Measurement of the impact of compounds on cell viability was performed using an Alamar Blue assay. Briefly, 15 pL Alamar Blue reagent was added to cells and fluorescence at 590 nm was measured after 3-5 h incubation at 37° C., 5% CO₂ using a fluorometer.

Data Analysis

For single agent (mono) treatment, raw data were converted into percent cell viability relative to the 0.1% DMSO control and positive control (10 μM staurosporine), which were set to 100% and 0%, respectively. IC₅₀ calculation was performed using GraphPad Prism software with a variable slope sigmoidal response fitting model using 0% cell growth as bottom or no constraint (as indicated) or 100% cel growth as top constraint. For combination treatments, combination compound concentrations tested were based on multiples of IC₅₀ values generated by mono treatment of cells. Raw data were converted into percent cel viability relative to the 0.1% DMSO control and positive control (10 μM staurosporine), set to 100% and 0%, respectively. Cell viability was converted into fraction affected ((100-cell viability)/100). Fraction affected data were compared to expected values according to the Bliss Independence model (E1+2=E1+E2-E1×E2).

Study KRS018-01-b (DiscoverX)

Test compounds dissolved in DMSO were profiled in a commercially-available tumour microenvironment (TME) model system consisting of PD-L1-expressing HT29 colorectal adenocarcinoma cells, primary stromal fibroblasts and PBMCs in which immune cel responses are suppressed by the presence of the cancer cells. Co-cultured cells were exposed to 2.5, 5, 10, 20 μM of test compound or DMSO as a control and stimulated with SAg for 48 h. The activity profile in the co-culture system was determined using ELISA endpoint assays to detect modulation of protein markers relevant to immune tolerance, inflammation, angiogenesis and matrix remodelling, and sulforhodamine B (SRB) and alamar blue assays to measure the viability of adherent colorectal adenocarcinoma cells and fibroblasts, and PBMCs, respectively. Assay measurements with values significantly different from vehicle controls (p<0.01) that were outside the inter-assay variation of controls (significance envelope) and that had an effect size >20% (log₁₀ ratio>0.1) in comparison to the DMSO vehicle control were considered significant.

Results

Proteosome Inhibitors

Velcade (Bortezomib):

The effects on the growth of Multiple Myeloma (MM) cancer cells of the HDAC inhibitor Compound A in combination with Velcade were tested in 6 cell lines in two independent studies (013_070_210814, 013_051_140814, Karus and 10922, ProQinase).

CI indexation suggested a synergistic effect at several combination concentrations on the growth inhibition of MM1.R cells (FIG. 1). Potentiation of Compound A-mediated growth inhibition in the presence of increasing concentrations of Velcade was observed in KMS-12-BM, RPMI-8226 and U266 cells (FIG. 2A) and in OPM-2 and to a limited extent in LP-1 cells (data not shown). Bliss independence analysis (across all concentrations tested) suggested a synergistic effect on the growth inhibition of KMS-12-BM, RPMI-8226 and U266 cel lines at some combination concentrations tested when combining Compound A & Velcade.

Kyprolis (Carfilzomib):

The effects on the growth of MM cells of Compound A in combination with Kyprolis were tested in 5 cel lines (10922). Potentiation of Compound A-mediated growth inhibition in the presence of increasing concentrations of Kyprolis was observed in KMS-12-BM, RPMI-8226, U266, OPM-2 and LP-1 cells (FIG. 3A). Bliss independence analysis (across all concentrations tested) suggested a synergistic effect on the growth inhibition of KMS-12-BM, RPMI-8226, U266, OPM-2 and to a limited extent LP-1 cell lines at some combination concentrations tested when combining Compound A and Kyprolis.

Immunomodulatory (IMID) Agents:

Revlimid (Lenalidomide):

The effects on the growth of MM cells of Compound A in combination with Revlimid were tested. Bliss independence analysis (across all concentrations tested) suggested a modest synergistic effect on the growth inhibition of KMS-12-BM and RPMI-8226 cell lines at some combination concentrations tested when combining Compound A & Revlimid.

Imnovid (Pomalidomide):

The effects on the growth of MM cells of Compound A in combination with Imnovid were tested in 5 cell lines (10922). Bliss independence analysis (across all concentrations tested) suggested a modest synergistic effect on the growth inhibition of KMS-12-BM and RPMI-8226 cell lines at some combination concentrations tested when combining Compound A & Imnovid.

Anti-PD-1 Monoclonal Antibody (Nivolumab):

The potential for Compound A to modulate immune responses in the tumour microenvironment (TME) were tested in a commercially-available TME model system consisting of PD-L1-expressing HT29 colorectal adenocarcinoma cells, primary stromal fibroblasts and PBMCs in which immune cell responses are suppressed by the presence of the cancer cells. When tested at concentrations of 2.5 to 10 μM, Compound A increased the levels of secreted granzyme-B, IFNγ, IL-10, IL-17A, IL-2, IL-6 and TNFα in a dose-dependent manner.

The activity of Compound A in the TME model was further tested in combination with therapeutic anti-PD1 monoclonal antibody nivolumab (Opdivo). Co-cultured cells were exposed to combinations of 2.5, 5, 10, and 20 μM of Compound A and 10, 100, 1000 and 10000 ng/mL nivolumab. Only the combination that used the highest concentration of both test agents (20 μM Compound A and 10000 ng/mL Opdivo) exhibited some PBMC cytotoxicity. In total, 15 of 16 combination concentrations modulated between 1 and 6 endpoint assay markers with values significantly different from both agents tested alone (and the monotherapy effect was >20% (log₁₀ ratio>0.1) in comparison to the DMSO vehicle control). The most effective combination was reported to be 10 μM Compound A and 10 ng/mL nivolumab, which decreased levels of collagen-I and collagen-Ill and increased granzyme-B, IFNγ, IL-17A and IL-6 secretion to levels statistically significant from both agents tested alone in comparison to the DMSO control. The increase in granzyme-B and IFNγ levels (suppressed by the presence of HT29 cancer cells), in the activity profile is consistent with the hypothesis of restoration of immune function in the immune-suppressed BioMAP TME model.

In Vivo Combination Efficacy Study

A. Summary

| | |
|---|---|
| Study title | Tumour growth delay study in male SCID mice |
| | bearing RPMI8226 tumours |
| Tumour Type | RPMI8226 |
| Test substance ID | Compound A |
| Formulation | 30/30/40 (v/v/v) propylene |
| | glycol/PEG400/aqueous (20%) HPβCD |
| Duration of dosing | 26 days (26 doses, animals culled on day 27) |

Male SCID mice were subcutaneously implanted with RPM18226 multiple myeloma cells. Once tumours were established (volume of ˜130 mm) treatment was commenced. Treatment was continued daily via PO for Compound A, IV twice a week for bortezomib and IP daily for lenalidomide/dexamethasone.

Compound A was well tolerated at all doses in both combination studies. All treatment groups had significantly smaller tumours than vehicle-treated controls at the end of the study.

Bioanalysis of plasma confirmed very low levels of compounds (<LLOQ for most compounds) at the end of the study. All compounds tested were detected in tumour tissue, albeit at low levels.

Western blot analysis demonstrated increased expression of acetylated tubulin in tumour tissue removed from animals treated with Compound A.

This study demonstrated the efficacy of Compound A in the RPM18226 model.

B. Method

A total of 115 male SCID mice (C.B-17/IcrHan@Hsd-Prkdc^{scid}) were purchased from Harlan (UK) and acclimatised for 7 days prior to study commencement. Animals were housed in IVC cages (5 per cage) with individual mice identified by tail mark. All animals were allowed free access to a standard certified commercial diet and sanitised water during the study. The holding room was maintained under standard conditions: 20-24° C., 40-70% humidity and a 12 h light/dark cycle.

RPMI cells (1×10⁷ in matrigel) were implanted subcutaneously into the rear dorsum of male SCID mice using a 25-gauge needle. When tumours were 100-150 mm³ animals were randomly assigned to treatment groups.

| | | | Dose | Dosing | Dosing |
|---|---|---|---|---|---|
| Group | n | Treatment | (mg/Kg) | route | schedule |
| 1 | 8 | Vehicle only | | p.o. | |
| 2 | 8 | Bortezomib | 0.5 | i.v. | Twice |
| | | | | | weekly |
| 3 | 8 | Lenalidomide + | 15 | i.p. | QD |
| | | Dexamethasone | | | |
| 4 | 8 | Compound A + | 100 | p.o | QD |
| | | Bortezomib† | | | |
| 5 | 8 | Compound A + | 200 | p.o | QD |
| | | Bortezomib† | | | |
| 9 | 8 | Compound A + | 100 | p.o | QD |
| | | Lenalidomide¥ + | | | |
| | | Dexamethasone§ | | | |

†Bortezomib dosed at 0.5 mg/kg, IV, twice weekly
¥Lenalidomide dosed at 15 mg/kg, IP, QD
§Dexamethasone dosed at 5 mg/kg, IP, QD

C. Results

Clinical Signs (Bortezomib Combination)

For the bortezomib combinations, Compound A was well tolerated with no significant weight change observed during the study.

| | | | Versus | |
|---|---|---|---|---|
| | Group mean | | vehicle | |
| Treatment | at day 26 | SD | (p-value) | Significance |
| Group 1 | 103.5 | 6.1 | — | — |
| Group 2 | 98.5 | 5.2 | 0.2794 | n/s |
| Group 4 | 102.4 | 6.4 | >0.999 | n/s |
| Group 5 | 99.0 | 4.5 | 0.4505 | n/s |

Tumour Volume (Bortezomib Combination)

Tumour growth in vehicle treated controls (Group 1) was as expected, with all tumours growing steadily throughout the treatment period. Animals treated with Compound A (Group 4 and 5) exhibited significant control of tumour after 8 days of treatment. Percent T/C values (treatment tumour volume/vehicle control tumour volume) at day 26 are shown below. The lower the T/C value is, the more efficacious the treatment combination.

| | Group mean | | Versus vehicle | | % T/C | Versus Bort | | % T/C |
|---|---|---|---|---|---|---|---|---|
| Treatment | at day 19 | SD | (p-value) | Sig. | value | (Group 2) | Sig. | value |
| Group 1 | 1451 | 100 | — | — | — | | | |
| Group 2 | 984 | 50 | <0.0001 | **** | 67.8 | — | — | — |
| Group 4 | 637 | 57 | <0.0001 | **** | 43.9 | <0.0001 | **** | 64.7 |
| Group 5 | 544 | 48 | <0.0001 | **** | 37.5 | <0.0001 | **** | 55.3 |
| Group 6 | 557 | 58 | <0.0001 | **** | 38.4 | <0.0001 | **** | 56.6 |

Mean Tumour volume at day 26. Statistical difference compared to vehicle only control or bortezomib alone treatment was analysed using a 2-way ANOVA.

1. A pharmaceutical composition comprising a combination of a compound of formula (I) or a pharmaceutically acceptable salt thereof, and at least one second agent selected from the group consisting of proteasome inhibitors, tumour immunotherapeutics or immunomodulatory agents, signal transduction pathway inhibitors, agents inhibiting the BCL2 family of proteins, agents inhibiting Mcl-1, poly (ADP-ribose) polymerase (PARP) Inhibitors, aromatase inhibitors, conventional cytotoxic agents or a miscellaneous agent selected from abiraterone, ARN-509 and MYC inhibitors;
wherein the compound of formula (I) is represented by: 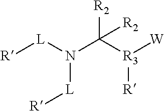
or a pharmaceutically acceptable salt thereof, wherein:
each R^{/} is independently selected from H and QR₁;
each Q is independently selected from a bond, CO, CO₂, NH, S, SO, SO₂ or O;
each R₁ is independently selected from H, C₁-C₁₀ alkyl, C₂-C₁₀ alkenyl, C₂-C₁₀ alkynyl, aryl, heteroaryl, C₁-C₁₀ cycloalkyl, halogen, C₁-C₁₀ alkylaryl, C₁-C₁₀ alkyl heteroaryl or C₁-C₁₀ heterocycloalkyl;
each L is independently selected from a 5 to 10-membered nitrogen-containing heteroaryl;
W is a zinc-binding group;
each R₂ is independently hydrogen or C₁ to C₆ alkyl; and
R₃ is an aryl or heteroaryl;
each aryl or heteroaryl may be substituted by up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃-acylamino, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl; and
each alkyl, alkenyl or alkynyl may be substituted with halogen, NH₂, NO₂ or hydroxyl.

2. A kit comprising at least one compound of Formula I as defined in claim 1 or a pharmaceutically acceptable salt thereof and at least one second agent selected from the group consisting of proteasome inhibitors, tumour immunotherapeutics or immunomodulatory agents, signal transduction pathway inhibitors, agents inhibiting the BCL2 family of proteins, agents inhibiting Md-1, poly (ADP-ribose) polymerase (PARP) Inhibitors, aromatase inhibitors, conventional cytotoxic agents or a miscellaneous agent selected from abiraterone, ARN-509 and MYC inhibitors, as a combined preparation for simultaneous, sequential or separate use in therapy.

3. A method of treating or preventing a condition in a patient comprising administering to the patient a therapeutically effective amount of at least one compound of Formula I as defined in claim 1 or a pharmaceutically acceptable salt thereof and at least one second agent selected from the group consisting of proteasome inhibitors, tumour immunotherapeutics or immunomodulatory agents, signal transduction pathway inhibitors, agents inhibiting the BCL2 family of proteins, agents inhibiting Md-1, poly (ADP-ribose) polymerase (PARP) Inhibitors, aromatase inhibitors, conventional cytotoxic agents or a miscellaneous agent selected from abiraterone, ARN-509 and MYC inhibitors.

4. A composition, kit or method according to any of claims 1 to 3, wherein at least one second agent is a proteasome inhibitor, preferably Bortezomib or Carfilzomib.

5. A composition, kit or method according to any of claims 1 to 3, wherein at least one second agent is a tumour immunotherapeutic or immunomodulatory agents, preferably a small molecule immunomodulator or an anti-PD-1 or anti-PD-L1 agent.

6. A composition, kit or method according to claim 5, wherein the tumour immunotherapeutic or immunomodulatory agent is Nivolumab, Lenalidomide or Pomalidomide.

7. A method according to any of claims 3 to 6, wherein the administration is separate, sequential or simultaneous.

8. The composition, kit or method according to any one of claims 1 to 5, wherein W is selected from: 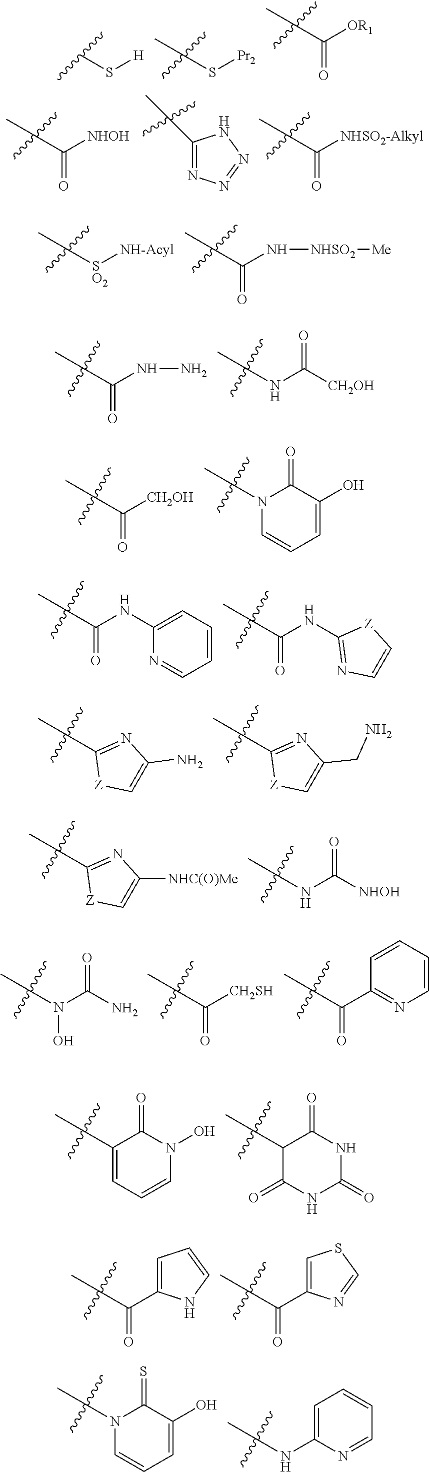 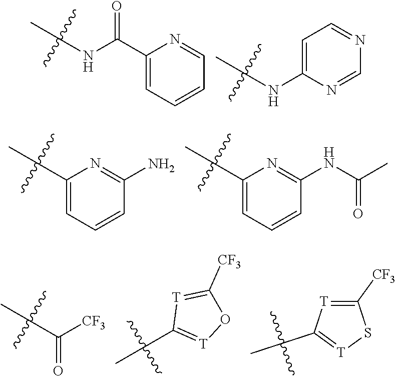
wherein R₁ is as defined in claim 1, Pr² is H or a thiol protecting group, Z is selected from O, S or NH and T is N or CH.

9. The composition, kit or method according to claim 8, wherein W is —CONHOH.

10. The composition, kit or method according to any preceding claim, wherein each L is independently selected from a 5 or 6-membered nitrogen-containing heteroaryl, which is optionally fused to a benzene.

11. The composition, kit or method according to any preceding claim, wherein in at least one, preferably both L groups, the atom that is directly bonded to the N is a carbon, and at least one nitrogen atom is directly bonded to said carbon.

12. The composition, kit or method according to any preceding claim, wherein L is independently selected from pyridinyl, pyrimidinyl, pyridazinyl, oxadiazolyl, pyrazolyl, thiadiazolyl, pyrazinyl, benzofused thiazolyl, benzofused oxazolyl or benzofused imidazolyl, preferably, L is independently selected from pyridyl and pyrazinyl.

13. The composition, kit or method according to any preceding claim, wherein at least one L group is pyridinyl, oxadiazolyl, pyrazolyl, thiadiazolyl, pyrazinyl, benzofused thiazolyl, benzofused oxazolyl or benzofused imidazolyl, preferably at least one L group is pyridyl or pyrazinyl.

14. The composition, kit or method according to any preceding claim, wherein R₃ is phenylene or phenylene substituted with a halogen.

15. The composition, kit or method according to any preceding claim, wherein at least one, preferably both, R₂ is/are H.

16. The composition, kit or method according to any preceding claim, wherein R′ that is attached to L is independently selected from H, C₁-C₁₀ alkyl or O—(C₁-C₁₀ alkyl), halogen, C₁-C₁₀ heterocycloalkyl, aryl, trifluoromethyl or heteroaryl.

17. The composition, kit or method according to any preceding claim, wherein at least one R′ is H, halogen, CF₃, C₁-C₆ alkyl, aryl optionally substituted with halogen, heteroaryl optionally substituted with halogen or heterocycloalkyl.

18. The composition, kit or method according to any preceding claim, wherein at least one of the R′ that is attached to L is heterocycloalkyl.

19. The composition, kit or method according to claim 18, wherein R′ attached to R₃ is hydrogen or halogen.

20. The composition, kit or method according to claim 18, wherein at least one R′ is C₁-C₆ alkyl optionally substituted with halogen, NH₂, NO₂ or hydroxyl.

21. The composition, kit or method according to claim 20, wherein at least one R′ is C₁-C₆ alkyl optionally substituted with halogen.

22. The composition, kit or method according to any preceding claim, wherein the compound of Formula (I) is as exemplified herein.

23. A combination according to claim 22, wherein the compound of Formula (I) is: 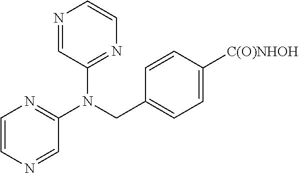
or a pharmaceutically acceptable salt thereof.

24. A composition, kit or method according to any preceding claim, wherein the second agent is selected from a proteasome inhibitor, an immunomodulatory or tumour immunotherapeutic agent and a signal transduction pathway inhibitor.

25. A pharmaceutical composition comprising a composition, kit or method as defined in any preceding claim, and a pharmaceutically acceptable excipient.

26. A composition, kit or method according to any preceding claim, for use in therapy.

27. A composition, kit or method according to any preceding claim, for use in the treatment or prevention of a condition mediated by histone deacetylase (HDAC).

28. A composition, kit or method according to claim 27, wherein the condition is cancer, cardiac hypertrophy, chronic heart failure, an inflammatory condition, a cardiovascular disease, a haemoglobinopathy, a thalassemia, a sickle cell disease, a CNS disorder, an autoimmune disease, diabetes, osteoporosis, MDS, benign prostatic hyperplasia, endometriosis, oral leukoplakia, a genetically related metabolic disorder, an infection, Rubens-Taybi, fragile X syndrome, or alpha-1 antitrypsin deficiency.

29. A composition, kit or method according to claim 27 or claim 28, wherein the condition is chronic lymphocytic leukaemia, breast cancer, prostate cancer, ovarian cancer, mesothelioma, T-cell lymphoma, cardiac hypertrophy, chronic heart failure, a skin inflammatory condition (in particular psoriasis, acne or eczema), a musculoskeletal inflammatory condition (in particular rheumatoid arthritis, juvenile rheumatoid arthritis, ankylosing spondylitis or osteoarthritis), or an inflammatory condition of the gastrointestinal tract (in particular inflammatory bowel disease, Crohn's disease, ulcerative colitis, or irritable bowel syndrome).

30. A composition, kit or method according to any preceding claim, for use in the treatment of cancer, preferably multiple myeloma.

31. A composition, kit or method according to any one of claims 1 to 23, for use in the treatment of solid tumours or haematological cancers.

32. A composition, kit or method according to any of claims 1 to 23, for use in accelerating wound healing, protecting hair follicles, or as an immunosuppressant.
